SEQUENCE LISTING (134 sequences)

SEQ ID NO: 1 - protein, 121 aa, synthetic construct
DVQLQESGPGLVKPSQSLSLTCSVSGYSITSGYYWSWIRQFPGNKLEWMGYISNDGSNKYKPSLKNRISITRDTSKNQFFLKLKSVTTEDTATYYCARFPLINYDPWLAYWGQGTLVTVSA

SEQ ID NO: 2 - protein, 117 aa, synthetic construct
EVQLQQSGPELEKPGASVKISCKASGYSFIGYSMNWVRQSDGKSLEWIGNIDPYYGGTSYNLKFKGKATLTVDKSSSTAYMQLKSLTSEDSAVYYCARLGRGYFDYWGQGTTLTVSS

SEQ ID NO: 3 - protein, 121 aa, synthetic construct
DVQLQESGPGLVKPSQSLSLTCSVTGYSITSGYYWNWIRQFPGNKLEWMGYIRNDGSNNYNPSLKNRISITRDTSKNQFFLKLNSVTTEDTATYYCARFPLINYDPWFAYWGQGTLVTVSA

SEQ ID NO: 4 - protein, 121 aa, synthetic construct
DVQLQESGPGLVKPSQSLSLTCSVSGYSITSGYYWNWIRQFPGNKLEWMGYISNDGNNKYKPSLKNRISITRDTSKNQFFLKLNSVTAEDTATYYCARFPLINYDPWLAYWGQGTLVTVSA

SEQ ID NO: 5 - protein, 120 aa, synthetic construct
EVQLQQSGPDLVKPGASVKISCKASGYSFTGYYMHWVKQSHGKSLEWIGRVYPNNGGTSYNQKFKGKAILTVDKSSTTAYMELRSLTSEDSAVYYCALIHYYGFYSMDYWGQGTSVTVSS

SEQ ID NO: 6 - protein, 121 aa, synthetic construct
DVQLQESGPGLVKPSQSLSLTCSVSGYSITSGYYWNWIRQFPGNKLEWMGYISNDGSNKYKPSLKNRISITRDTSKNQFFLKLNSVTTEDTATYYCARFPLINYDPWLAYWGQGTLVTVSA

SEQ ID NO: 7 - protein, 112 aa, synthetic construct
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPWTFGGGTKLEIN

SEQ ID NO: 8 - protein, 107 aa, synthetic construct
DIVLTQSPATLSVTPGDSVSLSCRASQSITNNLHWYQQKSHESPRLLIKSASQSISGIPSRFSGSGSGTDFTLSINSVETEDFGMYFCQQSNSWPHTFGGGTKLEIK

SEQ ID NO: 9 - protein, 112 aa, synthetic construct
DVLMTQAPLSLPVSLGDQASISCRSSQNVVHRNGNTYLEWYLQKPGQSPKLLIYKVSNRFSGVPDRFSGSGSGTDFTLKISRVEAEDLGVYYCFQGSHVPWTFGGGTKLEIK

SEQ ID NO: 10 - protein, 107 aa, synthetic construct
ETTVTQSPASLSVATGEKVTIRCITSTDIDDDMNWYQQKPGEPPKLLISEANTLRPGVPSRFSSSGYGTDFVFTIENTLSEDVADYYCLQSYNMPLTFGAGTTLELK

SEQ ID NO: 11 - protein, 112 aa, synthetic construct
DVLMTQTPLSLPVSLGDQASISCRSSQNVVHRNGNTYLEWYLQKPGQSPKLLIYKVSNRFSGVPDRFSGSGSGTDFTLKISRVEAEDLGVYYCFQGSHVPWTFGGGTKLEIK

SEQ ID NO: 12 - protein, 9 aa, synthetic construct
GYSITSGYY

SEQ ID NO: 13 - protein, 8 aa, synthetic construct
GYSFIGYS

SEQ ID NO: 14 - protein, 8 aa, synthetic construct
GYSFTGYY

SEQ ID NO: 15 - protein, 7 aa, synthetic construct
ISNDGSN

SEQ ID NO: 16 - protein, 8 aa, synthetic construct
IDPYYGGT

SEQ ID NO: 17 - protein, 7 aa, synthetic construct
IRNDGSN

SEQ ID NO: 18 - protein, 7 aa, synthetic construct
ISNDGNN

SEQ ID NO: 19 - protein, 8 aa, synthetic construct
VYPNNGGT

SEQ ID NO: 20 - protein, 14 aa, synthetic construct
ARFPLINYDPWLAY

SEQ ID NO: 21 - protein, 10 aa, synthetic construct
ARLGRGYFDY

SEQ ID NO: 22 - protein, 14 aa, synthetic construct
ARFPLINYDPWFAY

SEQ ID NO: 23 - protein, 13 aa, synthetic construct
ALIHYYGFYSMDY

SEQ ID NO: 24 - protein, 11 aa, synthetic construct
QSIVHSNGNTY

SEQ ID NO: 25 - protein, 6 aa, synthetic construct
QSITNN

SEQ ID NO: 26 - protein, 11 aa, synthetic construct
QNVVHRNGNTY

SEQ ID NO: 27 - protein, 6 aa, synthetic construct
TDIDDD

SEQ ID NO: 28-30 - skipped (3 entries)

SEQ ID NO: 31 - protein, 9 aa, synthetic construct
FQGSHVPWT

SEQ ID NO: 32 - protein, 9 aa, synthetic construct
QQSNSWPHT

SEQ ID NO: 33 - protein, 9 aa, synthetic construct
LQSYNMPLT

SEQ ID NO: 34 - DNA, 363 bp, synthetic construct
  source 1..363: mol_type = other DNA
gacgtgcagctgcaggagagcggtcctggcctggtgaagcctagccaatctctgagcctgacctgcagcgtgtccggatattccatcaccagcggctattactggtcctggatcagacagtttccaggcaacaagctggagtggatgggctacatcagcaacgacggcagcaacaagtacaagcccagcctgaaaaacagaatcagtattacaagagatacatctaagaaccagttcttcctgaagcttaaaagcgtgacaaccgaggacaccgccacctactactgcgcccggttccctctgatcaactacgacccctggctggcctactggggccagggcacactggttacagtgtccgcc

SEQ ID NO: 35 - DNA, 336 bp, synthetic construct
  source 1..336: mol_type = other DNA
gatgtcctgatgacccagacccctctcagcctgccagtgtccctgggcgaccaggccagcatcagctgcagaagcagccagtccatcgtgcatagcaatggaaatacctacctggaatggtatctgcaaaagcctggacagagccctaagctgctgatctacaaagtgagcaaccggttcagcggagtgcccgacagattctctggctctggctctggcacagatttcaccctgaagatctctagggtggaagccgaggacctgggcgtgtactactgttttcagggcagccacgtcccttggaccttcggcggcggcaccaagctggaaatcaac

SEQ ID NO: 36 - DNA, 345 bp, synthetic construct
  source 1..345: mol_type = other DNA
cagttgcagcagagcggtcctgagctggaaaagccaggcgccagcgtgaagatcagctgtaaagcttctggctatagcttcatcggctacagcatgaactgggtgcggcagagcgatggcaagtctctggaatggattggcaacatcgacccttactacggcggtacaagctacaatctgaaattcaagggcaaggccaccctgacagtggacaagtctagcagcaccgcctacatgcagctgaagagcctgacctccgaggatagcgccgtctactactgcgccagactgggcagaggatatttcgactactggggccagggcaccaccctgaccgtgtccagc

SEQ ID NO: 37 - DNA, 327 bp, synthetic construct
  source 1..327: mol_type = other DNA
gatatcgtgctgacccagtctcctgctacactgagcgttacaccaggcgactccgtgtctctcagttgcagagccagccagtccatcacaaacaacctgcattggtatcagcaaaagagccacgaaagccccagactgctgatcaagagcgccagccagagcatcagcggcatccctagccggttcagcggctctggcagcggaaccgacttcaccctttccatcaattctgtggagaccgaggacttcggcatgtacttttgccagcaaagcaacagctggccccacacctttggaggcggaacaaaactggaaatcaagggcgga

SEQ ID NO: 38 - DNA, 363 bp, synthetic construct
  source 1..363: mol_type = other DNA
gacgtgcagctgcaggagagcggacctggactggttaagcctagccaaagcctgagcctgacctgcagcgtgacaggctacagcatcaccagcgggtattactggaactggatcagacagttccccggcaacaagctggaatggatgggctacatcagaaacgacggctccaacaactacaacccttctctgaaaaaccggatctctattaccagagatacctccaagaaccagtttttcctgaagctgaatagcgtcacgaccgaggacacagctacatactattgtgctcggtttccactgatcaactacgacccctggttcgcctactggggccagggcaccctggtgacggtgtccgcc

SEQ ID NO: 39 - DNA, 336 bp, synthetic construct
  source 1..336: mol_type = other DNA
gacgtgctgatgacacaggcccctctgtctctgcccgtcagcctcggcgaccaggccagcatcagctgcagaagcagccagaacgtggtgcatagaaatggcaatacctacctggaatggtatctgcagaaacctggccaatctccaaagctgctgatctacaaggtgtccaacagattcagcggcgtgcctgatagattcagcggcagcggatctggaacagacttcaccctgaagatcagcagagtggaagccgaggatctgggcgtgtactactgcttccagggcagccacgtgccctggaccttcggcggcggcaccaagctcgagatcaag

SEQ ID NO: 40 - DNA, 363 bp, synthetic construct
  source 1..363: mol_type = other DNA
gacgtgcagctgcaggagagcggccctggcctggttaagccctctcagagcctttctctgacctgcagcgtgaccggatacagcatcaccagcggctattactggaactggatcagacagtttccaggcaacaagctcgagtggatgggctatatccggaacgacggcagcaacaactacaacccaagcctgaaaaatagaatcagcatcacccgggacaccagcaagaaccagttcttcctgaagctgaacagcgtgaccacggaagataccgccacctactactgtgctagattccccctgattaactacgacccttggttcgcctactggggccagggcacactggtcaccgtgtccgcc

SEQ ID NO: 41 - DNA, 321 bp, synthetic construct
  source 1..321: mol_type = other DNA
gaaaccacagtgacacaaagccctgccagcctgtccgtggctacaggagagaaggtgaccatccggtgcatcaccagtacagatatcgacgacgatatgaactggtatcagcagaagcctggagaaccccccaagctgctgatctccgaggccaataccctgagacccggcgtgccttctaggttcagcagctctggttacggcacagacttcgtgttcaccatcgagaacacactgtctgaggatgtggccgactactactgcctgcaaagctacaatatgcctctgacctttggcgccggcacaaccctggaactgaaa

SEQ ID NO: 42 - DNA, 363 bp, synthetic construct
  source 1..363: mol_type = other DNA
gacgtgcagctgcaggagagcggacctggcctggtgaagcctagccagagcctgagcctgacatgttccgtgagcggctacagcatcaccagcggctactactggaactggattagacagtttcccggcaacaagctggagtggatgggatacatcagcaacgacggcaacaacaaatacaagccaagcctgaaaaaccggatctctatcacaagagataccagcaagaaccagtttttcctgaagctgaacagcgtgacagccgaggacaccgctacatactattgcgccagattccccctgatcaactacgacccctggctggcttattggggccaaggcaccctggttaccgtgtccgcc

SEQ ID NO: 43 - DNA, 336 bp, synthetic construct
  source 1..336: mol_type = other DNA
gacgtgctgatgacccagacccctctgagcctgcctgtgtccctgggcgatcaggcctctatctcctgcagaagctctcagtctatcgtgcatagcaacggcaatacctacctggaatggtatctgcaaaagcctggccagtctcctaagcttctgatctacaaggtgtctaatcggttcagcggagtccccgaccgcttcagcggcagcggcagcggcaccgacttcaccctgaaaatctcaagagtggaagccgaggatctgggcgtgtactactgcttccagggcagccacgtgccttggaccttcggcggcggcacaaagctggaaatcaat

SEQ ID NO: 44 - DNA, 360 bp, synthetic construct
  source 1..360: mol_type = other DNA
gaggtgcagctgcagcagagcggtcctgatctggtgaaacctggcgctagcgtgaagatcagctgcaaggcctctggatattcttttacaggctactacatgcactgggtcaagcagagccacggcaaaagcctggagtggatcggcagagtttaccccaacaacggcggcacaagctacaaccagaaattcaagggcaaggctatcctgaccgtggacaagtcctccaccaccgcctacatggaactgcggagcctgacatctgaggatagcgccgtgtactactgcgccctgattcactactacggcttctacagcatggactactggggccagggcaccagcgtgacagtgtccagc

SEQ ID NO: 45 - DNA, 336 bp, synthetic construct
  source 1..336: mol_type = other DNA
gacgtgctgatgacccagacccctctgagcctgcctgtgagcctcggcgaccaggccagcatctcttgtagaagctcacagtctatcgtgcatagcaacggaaatacctacctggaatggtatctgcaaaagcccggccaatcccctaagctgctgatctacaaggtgtctaatcggtttagcggagtgcccgataggttcagcggcagcggcagcggcaccgacttcaccctgaagatctctagagtggaagccgaggacctgggcgtgtactattgcttccagggctcccatgtgccatggaccttcggcggcggcacaaagctggagatcaac

SEQ ID NO: 46 - DNA, 363 bp, synthetic construct
  source 1..363: mol_type = other DNA
gatgtgcagctgcaagagagcggccctggcctggtcaagcctagccaaagcctgagcctgacctgcagcgtttccggctacagcatcacctctggctactactggaactggatcagacagttccctggaaataaactggaatggatgggatacatcagcaatgacggctctaacaagtacaagcccagcctcaagaacagaatcagcattacccgcgacacctccaagaaccagtttttcctgaagctgaacagcgtgaccacagaagataccgctacatactactgcgccagattccccctcatcaactacgacccctggctggcctattggggccagggaacactggtgacagtgtccgcc

SEQ ID NO: 47 - DNA, 336 bp, synthetic construct
  source 1..336: mol_type = other DNA
gacgtgctgatgacccagacccctctgtctctgcctgtgtccctgggcgaccaggcctctatcagctgtagaagcagccagaacgtggtgcatagaaacggcaacacctacctggagtggtatctgcagaaacctggccagagccctaagctgctgatctacaaggtgtctaatcggttcagcggagtgcccgaccggtttagcggcagcggctctggaacagatttcaccctgaaaatcagcagagtggaagccgaggacctgggcgtgtactattgcttccagggcagccacgtgccatggacattcggcggtggcaccaagctggagatcaag

SEQ ID NO: 48 - DNA, 363 bp, synthetic construct
  source 1..363: mol_type = other DNA
gacgtgcagctgcaggagagcggtcctggcctggtcaagcctagccagagcctgagccttacatgcagcgtgtccggctacagcatcacctctggatattactggtcctggatcagacagtttcctggcaacaagctggaatggatgggctacatcagcaacgacggctctaacaaatacaagcccagcctgaagaaccggatcagcattacaagagataccagcaagaaccagttcttcctgaaactgaaaagcgtcaccaccgaggatacagctacatactattgtgccagattccccctgatcaactacgacccctggctggcctactggggacaaggcaccctggtgacagtgtccgcc

SEQ ID NO: 49 - DNA, 336 bp, synthetic construct
  source 1..336: mol_type = other DNA
gacgtgctgatgacccagacccctctgtctctgcctgtgtccttgggcgaccaggccagcatctcatgcagaagcagccagaacgtggtgcatagaaacggcaatacctacctggaatggtatctgcaaaagcccggccagtctccaaagctgctcatctacaaggtgtctaataggtttagcggcgttcctgatagattcagcggcagcggcagcggcacagacttcaccctgaagatctctagagtggaagccgaggacctgggcgtgtactactgcttccagggcagccacgtgccttggaccttcggcggaggaaccaagctggagatcaag

SEQ ID NO: 50 - DNA, 1479 bp, synthetic construct
  source 1..1479: mol_type = other DNA
atggccctgcctgtgaccgccctgctgctgcctctggccctgctgctgcacgccgctcgccccgacgtgctgatgacacaggcccctctgtctctgcccgtcagcctcggcgaccaggccagcatcagctgcagaagcagccagaacgtggtgcatagaaatggcaatacctacctggaatggtatctgcagaaacctggccaatctccaaagctgctgatctacaaggtgtccaacagattcagcggcgtgcctgatagattcagcggcagcggatctggaacagacttcaccctgaagatcagcagagtggaagccgaggatctgggcgtgtactactgcttccagggcagccacgtgccctggaccttcggcggcggcaccaagctcgagatcaagggtggcggcggaagcggaggcggcggctctggcggaggcggcagcgacgtgcagctgcaggagagcggacctggactggttaagcctagccaaagcctgagcctgacctgcagcgtgacaggctacagcatcaccagcgggtattactggaactggatcagacagttccccggcaacaagctggaatggatgggctacatcagaaacgacggctccaacaactacaacccttctctgaaaaaccggatctctattaccagagatacctccaagaaccagtttttcctgaagctgaatagcgtcacgaccgaggacacagctacatactattgtgctcggtttccactgatcaactacgacccctggttcgcctactggggccagggcaccctggtgacggtgtccgccacaaccacccctgcccctagacctcctacccctgctccaaccatcgccagccagcccctgtctctgcggcctgaagcttgcagacccgctgctggcggcgccgtgcacacgaggggactggacttcgcctgcgacatctacatttgggcccctctggccggcacctgcggcgtgctgctgctgagcctggtcatcacactgtactgcaagagaggcagaaaaaagctgctgtacatcttcaagcagccttttatgcggcccgtgcagaccacacaggaggaagatggctgctcttgtagattccccgaggaagaggaaggcggatgtgaactgagagtgaagttcagcagaagcgccgatgcccctgcctaccagcagggacagaaccagctgtacaacgagctgaacctgggcagacgggaagagtacgacgtgctggataagcggcggggtcgggaccccgagatgggaggcaagcctagaaggaagaacccccaagagggcctttataatgagctgcagaaagataagatggccgaagcctacagcgagatcggcatgaagggcgagagacgcagaggcaaaggccacgacggcctgtaccaaggcctctccaccgccacaaaggacacctacgacgccctgcacatgcaggccctgcctccaagatga

SEQ ID NO: 51 - protein, 492 aa, synthetic construct
MALPVTALLLPLALLLHAARPDVLMTQAPLSLPVSLGDQASISCRSSQNVVHRNGNTYLEWYLQKPGQSPKLLIYKVSNRFSGVPDRFSGSGSGTDFTLKISRVEAEDLGVYYCFQGSHVPWTFGGGTKLEIKGGGGSGGGGSGGGGSDVQLQESGPGLVKPSQSLSLTCSVTGYSITSGYYWNWIRQFPGNKLEWMGYIRNDGSNNYNPSLKNRISITRDTSKNQFFLKLNSVTTEDTATYYCARFPLINYDPWFAYWGQGTLVTVSATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 21 aa, synthetic construct
MALPVTALLLPLALLLHAARP

SEQ ID NO: 53 - protein, 248 aa, synthetic construct
DVLMTQAPLSLPVSLGDQASISCRSSQNVVHRNGNTYLEWYLQKPGQSPKLLIYKVSNRFSGVPDRFSGSGSGTDFTLKISRVEAEDLGVYYCFQGSHVPWTFGGGTKLEIKGGGGSGGGGSGGGGSDVQLQESGPGLVKPSQSLSLTCSVTGYSITSGYYWNWIRQFPGNKLEWMGYIRNDGSNNYNPSLKNRISITRDTSKNQFFLKLNSVTTEDTATYYCARFPLINYDPWFAYWGQGTLVTVSA

SEQ ID NO: 54 - protein, 15 aa, synthetic construct
GGGGSGGGGSGGGGS

SEQ ID NO: 55 - protein, 45 aa, synthetic construct
TTTPAPRPPTPAPTIASQPLSLRPEACRPAAGGAVHTRGLDFACD

SEQ ID NO: 56 - protein, 24 aa, synthetic construct
IYIWAPLAGTCGVLLLSLVITLYC

SEQ ID NO: 57 - protein, 42 aa, synthetic construct
KRGRKKLLYIFKQPFMRPVQTTQEEDGCSCRFPEEEEGGCEL

SEQ ID NO: 58 - protein, 492 aa, synthetic construct; identical to SEQ ID NO: 51

SEQ ID NO: 59 - DNA, 1464 bp, synthetic construct
  source 1..1464: mol_type = other DNA
atggccctgcctgtgaccgccctgctgctgcctctggccctgctgctgcacgccgctcgccccgacgtgctgatgacacaggcccctctgtctctgcccgtcagcctcggcgaccaggccagcatcagctgcagaagcagccagaacgtggtgcatagaaatggcaatacctacctggaatggtatctgcagaaacctggccaatctccaaagctgctgatctacaaggtgtccaacagattcagcggcgtgcctgatagattcagcggcagcggatctggaacagacttcaccctgaagatcagcagagtggaagccgaggatctgggcgtgtactactgcttccagggcagccacgtgccctggaccttcggcggcggcaccaagctcgagatcaagggtggcggcggaagcggaggcggcggctctggcggaggcggcagcgacgtgcagctgcaggagagcggacctggactggttaagcctagccaaagcctgagcctgacctgcagcgtgacaggctacagcatcaccagcgggtattactggaactggatcagacagttccccggcaacaagctggaatggatgggctacatcagaaacgacggctccaacaactacaacccttctctgaaaaaccggatctctattaccagagatacctccaagaaccagtttttcctgaagctgaatagcgtcacgaccgaggacacagctacatactattgtgctcggtttccactgatcaactacgacccctggttcgcctactggggccagggcaccctggtgaccgtgtccgccacaaccacccctgcccctagacctcctacccctgctccaaccatcgccagccagcccctgtctctgcggcctgaagcttgcagacccgctgctggcggcgccgtgcacacgaggggactggacttcgcctgcgacatctacatttgggcccctctggccggcacctgcggcgtgctgctgctgagcctggtcatcacactgtactgccggagggaccagaggctgccccccgatgcccacaagccccctgggggaggcagtttccggacccccatccaagaggagcaggccgacgcccactccaccctggccaagatcagagtgaagttcagcagaagcgccgatgcccctgcctaccagcagggacagaaccagctgtacaacgagctgaacctgggcagacgggaagagtacgacgtgctggataagcggcggggtcgggaccccgagatgggaggcaagcctagaaggaagaacccccaagagggcctttataatgagctgcagaaagataagatggccgaagcctacagcgagatcggcatgaagggcgagagacgcagaggcaaaggccacgacggcctgtaccaaggcctctccaccgccacaaaggacacctacgacgccctgcacatgcaggccctgcctccaagatga

SEQ ID NO: 60 - protein, 487 aa, synthetic construct
MALPVTALLLPLALLLHAARPDVLMTQAPLSLPVSLGDQASISCRSSQNVVHRNGNTYLEWYLQKPGQSPKLLIYKVSNRFSGVPDRFSGSGSGTDFTLKISRVEAEDLGVYYCFQGSHVPWTFGGGTKLEIKGGGGSGGGGSGGGGSDVQLQESGPGLVKPSQSLSLTCSVTGYSITSGYYWNWIRQFPGNKLEWMGYIRNDGSNNYNPSLKNRISITRDTSKNQFFLKLNSVTTEDTATYYCARFPLINYDPWFAYWGQGTLVTVSATTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - DNA, 1453 bp, synthetic construct
  source 1..1453: mol_type = other DNA
atggccctgcctgtgaccgccctgctgctgcctctggccctgctgctgcacgccgctcgccccgacgtgctgatgacacaggcccctctgtctctgcccgtcagcctcggcgaccaggccagcatcagctgcagaagcagccagaacgtggtgcatagaaatggcaatacctacctggaatggtatctgcagaaacctggccaatctccaaagctgctgatctacaaggtgtccaacagattcagcggcgtgcctgatagattcagcggcagcggatctggaacagacttcaccctgaagatcagcagagtggaagccgaggatctgggcgtgtactactgcttccagggcagccacgtgccctggaccttcggcggcggcaccaagctcgagatcaagggtggcggcggaagcggaggcggcggctctggcggaggcggcagcgacgtgcagctgcaggagagcggacctggactggttaagcctagccaaagcctgagcctgacctgcagcgtgacaggctacagcatcaccagcgggtattactggaactggatcagacagttccccggcaacaagctggaatggatgggctacatcagaaacgacggctccaacaactacaacccttctctgaaaaaccggatctctattaccagagatacctccaagaaccagtttttcctgaagctgaatagcgtcacgaccgaggacacagctacatactattgtgctcggtttccactgatcaactacgacccctggttcgcctactggggccagggcaccctggtgacggtgagcgctattgaagtgatgtacccaccaccatacctggacaacgagaagagcaacgggactattatccatgtgaaggggaaacacctgtgcccaagcccactgttccccggcccttccaagcccttttgggtgctggtggtggtgggcggcgtcctggcttgttactctctgctggtcactgtggctttcatcatcttctgggtcaagaggagccgcctgctgcactccgactacatgaacatgacccctagacggcccggaccaacaaggaagcactaccagccctatgccccccctagggatttcgccgcctatagagtgaagttcagcagaagcgccgatgcccctgcctaccagcagggacagaaccagctgtacaacgagctgaacctgggcagacgggaagagtacgacgtgctggataagcggcggggtcgggaccccgagatgggaggcaagcctagaaggaagaacccccaagagggcctttataatgagctgcagaaagataagatggccgaagcctacagcgagatcggcatgaagggcgagagacgcagaggcaaaggccacgacggcctgtaccaaggcctctccaccgccacaaaggacacctacgacgccctgcacatgcaggccctgcctccaagat

SEQ ID NO: 62 - protein, 484 aa, synthetic construct
MALPVTALLLPLALLLHAARPDVLMTQAPLSLPVSLGDQASISCRSSQNVVHRNGNTYLEWYLQKPGQSPKLLIYKVSNRFSGVPDRFSGSGSGTDFTLKISRVEAEDLGVYYCFQGSHVPWTFGGGTKLEIKGGGGSGGGGSGGGGSDVQLQESGPGLVKPSQSLSLTCSVTGYSITSGYYWNWIRQFPGNKLEWMGYIRNDGSNNYNPSLKNRISITRDTSKNQFFLKLNSVTTEDTATYYCARFPLINYDPWFAYWGQGTLVTVSAIEVMYPPPYLDNEKSNGTIIHVKGKHLCPSPLFPGPSKPFWVLVVVGGVLACYSLLVTVAFIIFWVKRSRLLHSDYMNMTPRRPGPTRKHYQPYAPPRDFAAYRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - DNA, 1453 bp, synthetic construct
  source 1..1453: mol_type = other DNA
atggccctgcctgtgaccgccctgctgctgcctctggccctgctgctgcacgccgctcgccccgacgtgctgatgacacaggcccctctgtctctgcccgtcagcctcggcgaccaggccagcatcagctgcagaagcagccagaacgtggtgcatagaaatggcaatacctacctggaatggtatctgcagaaacctggccaatctccaaagctgctgatctacaaggtgtccaacagattcagcggcgtgcctgatagattcagcggcagcggatctggaacagacttcaccctgaagatcagcagagtggaagccgaggatctgggcgtgtactactgcttccagggcagccacgtgccctggaccttcggcggcggcaccaagctcgagatcaagggtggcggcggaagcggaggcggcggctctggcggaggcggcagcgacgtgcagctgcaggagagcggacctggactggttaagcctagccaaagcctgagcctgacctgcagcgtgacaggctacagcatcaccagcgggtattactggaactggatcagacagttccccggcaacaagctggaatggatgggctacatcagaaacgacggctccaacaactacaacccttctctgaaaaaccggatctctattaccagagatacctccaagaaccagtttttcctgaagctgaatagcgtcacgaccgaggacacagctacatactattgtgctcggtttccactgatcaactacgacccctggttcgcctactggggccagggcaccctggtgacggtgagcgctattgaagtgatgtacccaccaccatacctggacaacgagaagagcaacgggactattatccatgtgaaggggaaacacctgtgcccaagcccactgttccccggcccttccaagcccttttgggtgctggtggtggtgggcggcgtcctggcttgttactctctgctggtcactgtggctttcatcatcttctgggtccggagggaccagaggctgccccccgatgcccacaagccccctgggggaggcagtttccggacccccatccaagaggagcaggccgacgcccactccaccctggccaagatcagagtgaagttcagcagaagcgccgatgcccctgcctaccagcagggacagaaccagctgtacaacgagctgaacctgggcagacgggaagagtacgacgtgctggataagcggcggggtcgggaccccgagatgggaggcaagcctagaaggaagaacccccaagagggcctttataatgagctgcagaaagataagatggccgaagcctacagcgagatcggcatgaagggcgagagacgcagaggcaaaggccacgacggcctgtaccaaggcctctccaccgccacaaaggacacctacgacgccctgcacatgcaggccctgcctccaagat

SEQ ID NO: 64 - protein, 484 aa, synthetic construct
MALPVTALLLPLALLLHAARPDVLMTQAPLSLPVSLGDQASISCRSSQNVVHRNGNTYLEWYLQKPGQSPKLLIYKVSNRFSGVPDRFSGSGSGTDFTLKISRVEAEDLGVYYCFQGSHVPWTFGGGTKLEIKGGGGSGGGGSGGGGSDVQLQESGPGLVKPSQSLSLTCSVTGYSITSGYYWNWIRQFPGNKLEWMGYIRNDGSNNYNPSLKNRISITRDTSKNQFFLKLNSVTTEDTATYYCARFPLINYDPWFAYWGQGTLVTVSAIEVMYPPPYLDNEKSNGTIIHVKGKHLCPSPLFPGPSKPFWVLVVVGGVLACYSLLVTVAFIIFWVRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - DNA, 1470 bp, synthetic construct
  source 1..1470: mol_type = other DNA
atggccctgcctgtgaccgccctgctgctgcctctggccctgctgctgcacgccgctcgccccgacgtgctgatgacacaggcccctctgtctctgcccgtcagcctcggcgaccaggccagcatcagctgcagaagcagccagaacgtggtgcatagaaatggcaatacctacctggaatggtatctgcagaaacctggccaatctccaaagctgctgatctacaaggtgtccaacagattcagcggcgtgcctgatagattcagcggcagcggatctggaacagacttcaccctgaagatcagcagagtggaagccgaggatctgggcgtgtactactgcttccagggcagccacgtgccctggaccttcggcggcggcaccaagctcgagatcaagggtggcggcggaagcggaggcggcggctctggcggaggcggcagcgacgtgcagctgcaggagagcggacctggactggttaagcctagccaaagcctgagcctgacctgcagcgtgacaggctacagcatcaccagcgggtattactggaactggatcagacagttccccggcaacaagctggaatggatgggctacatcagaaacgacggctccaacaactacaacccttctctgaaaaaccggatctctattaccagagatacctccaagaaccagtttttcctgaagctgaatagcgtcacgaccgaggacacagctacatactattgtgctcggtttccactgatcaactacgacccctggttcgcctactggggccagggcaccctggtgacggtgagcgctattgaagtgatgtacccaccaccatacctggacaacgagaagagcaacgggactattatccatgtgaaggggaaacacctgtgcccaagcccactgttccccggcccttccaagcccttttgggtgctggtggtggtgggcggcgtcctggcttgttactctctgctggtcactgtggctttcatcatcttctgggtcaagagaggcagaaaaaagctgctgtacatcttcaagcagccttttatgcggcccgtgcagaccacacaggaggaagatggctgctcttgtagattccccgaggaagaggaaggcggatgtgaactgagagtgaagttcagcagaagcgccgatgcccctgcctaccagcagggacagaaccagctgtacaacgagctgaacctgggcagacgggaagagtacgacgtgctggataagcggcggggtcgggaccccgagatgggaggcaagcctagaaggaagaacccccaagagggcctttataatgagctgcagaaagataagatggccgaagcctacagcgagatcggcatgaagggcgagagacgcagaggcaaaggccacgacggcctgtaccaaggcctctccaccgccacaaaggacacctacgacgccctgcacatgcaggccctgcctccaagatga

SEQ ID NO: 66 - protein, 489 aa, synthetic construct
MALPVTALLLPLALLLHAARPDVLMTQAPLSLPVSLGDQASISCRSSQNVVHRNGNTYLEWYLQKPGQSPKLLIYKVSNRFSGVPDRFSGSGSGTDFTLKISRVEAEDLGVYYCFQGSHVPWTFGGGTKLEIKGGGGSGGGGSGGGGSDVQLQESGPGLVKPSQSLSLTCSVTGYSITSGYYWNWIRQFPGNKLEWMGYIRNDGSNNYNPSLKNRISITRDTSKNQFFLKLNSVTTEDTATYYCARFPLINYDPWFAYWGQGTLVTVSAIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 8 aa, synthetic construct
GYSITSGY

SEQ ID NO: 68 - protein, 11 aa, synthetic construct
GYSITSGYYWS

SEQ ID NO: 69 - protein, 6 aa, synthetic construct
SGYYWS

SEQ ID NO: 70 - protein, 7 aa, synthetic construct
TSGYYWS

SEQ ID NO: 71 - protein, 5 aa, synthetic construct
SNDGS

SEQ ID NO: 72 - protein, 9 aa, synthetic construct
YISNDGSNK

SEQ ID NO: 73 - protein, 16 aa, synthetic construct
YISNDGSNKYKPSLKN

SEQ ID NO: 74 - protein, 12 aa, synthetic construct
WMGYISNDGSNK

SEQ ID NO: 75 - protein, 12 aa, synthetic construct
FPLINYDPWLAY

SEQ ID NO: 76 - protein, 13 aa, synthetic construct
ARFPLINYDPWLA

SEQ ID NO: 77 - protein, 16 aa, synthetic construct
RSSQSIVHSNGNTYLE

SEQ ID NO: 78 - protein, 12 aa, synthetic construct
VHSNGNTYLEWY

SEQ ID NO: 79 - protein, 17 aa, synthetic construct
LEWYLQKPGQSPKLLIY

SEQ ID NO: 80 - protein, 7 aa, synthetic construct
KVSNRFS

SEQ ID NO: 81 - protein, 10 aa, synthetic construct
LLIYKVSNRF

SEQ ID NO: 82 - skipped

SEQ ID NO: 83 - protein, 8 aa, synthetic construct
FQGSHVPW

SEQ ID NO: 84 - protein, 7 aa, synthetic construct
GYSFIGY

SEQ ID NO: 85 - protein, 10 aa, synthetic construct
GYSFIGYSMN

SEQ ID NO: 86 - protein, 5 aa, synthetic construct
GYSMN

SEQ ID NO: 87 - protein, 6 aa, synthetic construct
IGYSMN

SEQ ID NO: 88 - protein, 8 aa, synthetic construct
GYSFIGYS

SEQ ID NO: 89 - protein, 6 aa, synthetic construct
DPYYGG

SEQ ID NO: 90 - protein, 10 aa, synthetic construct
NIDPYYGGTS

SEQ ID NO: 91 - protein, 17 aa, synthetic construct
NIDPYYGGTSYNLKFKG

SEQ ID NO: 92 - protein, 13 aa, synthetic construct
WIGNIDPYYGGTS

SEQ ID NO: 93 - protein, 8 aa, synthetic construct
LGRGYFDY

SEQ ID NO: 94 - protein, 9 aa, synthetic construct
ARLGRGYFD

SEQ ID NO: 95 - protein, 11 aa, synthetic construct
RASQSITNNLH

SEQ ID NO: 96 - protein, 7 aa, synthetic construct
TNNLHWY

SEQ ID NO: 97 - protein, 6 aa, synthetic construct
QSITNN

SEQ ID NO: 98 - protein, 7 aa, synthetic construct
SASQSIS

SEQ ID NO: 99 - protein, 10 aa, synthetic construct
LLIKSASQSI

SEQ ID NO: 100 - skipped

SEQ ID NO: 101 - protein, 8 aa, synthetic construct
QQSNSWPH

SEQ ID NO: 102 - protein, 11 aa, synthetic construct
GYSITSGYYWN

SEQ ID NO: 103 - protein, 6 aa, synthetic construct
SGYYWN

SEQ ID NO: 104 - protein, 7 aa, synthetic construct
TSGYYWN

SEQ ID NO: 105 - protein, 5 aa, synthetic construct
RNDGS

SEQ ID NO: 106 - protein, 9 aa, synthetic construct
YIRNDGSNN

SEQ ID NO: 107 - protein, 16 aa, synthetic construct
YIRNDGSNNYNPSLKN

SEQ ID NO: 108 - protein, 12 aa, synthetic construct
WMGYIRNDGSNN

SEQ ID NO: 109 - protein, 12 aa, synthetic construct
FPLINYDPWFAY

SEQ ID NO: 110 - protein, 13 aa, synthetic construct
ARFPLINYDPWFA

SEQ ID NO: 111 - protein, 16 aa, synthetic construct
RSSQNVVHRNGNTYLE

SEQ ID NO: 112 - protein, 12 aa, synthetic construct
VHRNGNTYLEWY

SEQ ID NO: 113 - protein, 11 aa, synthetic construct
ITSTDIDDDMN

SEQ ID NO: 114 - protein, 7 aa, synthetic construct
DDDMNWY

SEQ ID NO: 115 - protein, 7 aa, synthetic construct
EANTLRP

SEQ ID NO: 116 - protein, 10 aa, synthetic construct
LLISEANTLR

SEQ ID NO: 117 - skipped

SEQ ID NO: 118 - protein, 8 aa, synthetic construct
LQSYNMPL

SEQ ID NO: 119 - protein, 5 aa, synthetic construct
SNDGN

SEQ ID NO: 120 - protein, 9 aa, synthetic construct
YISNDGNNK

SEQ ID NO: 121 - protein, 16 aa, synthetic construct
YISNDGNNKYKPSLKN

SEQ ID NO: 122 - protein, 12 aa, synthetic construct
WMGYISNDGNNK

SEQ ID NO: 123 - protein, 7 aa, synthetic construct
GYSFTGY

SEQ ID NO: 124 - protein, 10 aa, synthetic construct
GYSFTGYYMH

SEQ ID NO: 125 - protein, 5 aa, synthetic construct
GYYMH

SEQ ID NO: 126 - protein, 6 aa, synthetic construct
TGYYMH

SEQ ID NO: 127 - protein, 6 aa, synthetic construct
YPNNGG

SEQ ID NO: 128 - protein, 10 aa, synthetic construct
RVYPNNGGTS

SEQ ID NO: 129 - protein, 17 aa, synthetic construct
RVYPNNGGTSYNQKFKG

SEQ ID NO: 130 - protein, 13 aa, synthetic construct
WIGRVYPNNGGTS

SEQ ID NO: 131 - protein, 12 aa, synthetic construct
ALIHYYGFYSMD

SEQ ID NO: 132 - protein, 8 aa, synthetic construct
DYKDDDDK

SEQ ID NO: 133 - protein, 6 aa, synthetic construct
HHHHHH

SEQ ID NO: 134 - protein, 9 aa, synthetic construct
YPYDVPDYA

(19) United States
(12) Patent Application Publication
(10) Pub. No.: US 2025/0222105 A1
(43) Pub. Date: Jul. 10, 2025

(54) ANTI-RORI ANTIBODY AND CHIMERIC ANTIGEN RECEPTOR AND METHODS OF USE THEREOF

(71) Applicant: BOARD OF REGENTS, THE UNIVERSITY OF TEXAS SYSTEM, Austin, TX (US)

(72) Inventors: Sattva S. NEELAPU, Houston, TX (US); Jinsheng WENG, Houston, TX (US); Yong PAN, Houston, TX (US)

(21) Appl. No.: 18/850,909

(22) PCT Filed: Mar. 27, 2023
(86) PCT No.: PCT/US2023/064982

§ 371 Date: Sep. 25, 2024

Related U.S. Application Data
(60) Provisional application No. 63/323,532, filed on Mar. 25, 2022.

(51) Int. Cl. A61K 40/42; A61K 40/11; A61K 40/31; A61P 35/00; C07K 14/725; C07K 16/28

(52) U.S. Cl. CPC A61K 40/421; A61K 40/11; A61K 40/31; A61P 35/00; C07K 14/7051; C07K 16/2803; C07K 2317/24; C07K 2317/569

(57) ABSTRACT

Provided herein are antibodies and antigen binding portions thereof that specifically bind receptor tyrosine kinase like orphan receptor 1 (ROR1), various compositions of such antibodies or antigen binding portions thereof, recombinant nucleic acids encoding the antibodies and antigen binding portions thereof, and methods of using the antibodies or antigen-binding portions thereof in cancer therapeutics and diagnostics.

### CROSS-REFERENCE TO RELATED APPLICATIONS

The present application claims priority to U.S. Provisional Application No. 63/323,532 filed Mar. 25, 2022, the full disclosure of which is incorporated by reference in its entirety for all purposes.

### BACKGROUND

Chimeric antigen receptor (CAR)-modified T cell therapy targeting CD19 induces high response rates in the majority of patients with refractory B-cell malignancies including follicular lymphoma (FL), mantle cell lymphoma (MCL), diffuse large B-cell lymphoma (DLBCL), and chronic lymphocytic leukemia (CLL). A subset of these patients experiences long-term remissions suggesting adoptive T-cell therapy could be curative in patients refractory to existing therapies. However, >50% of patients relapse or progress after CD19 CAR T-cell therapy, and a major cause of failure appears to be loss of CD19 expression on the tumor cells. Therefore, novel targets for adoptive T-cell therapeutic approaches are needed to further improve clinical outcome in these patients.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

In one aspect, provided herein are isolated antibodies or antigen-binding portions thereof that specifically bind to ROR1. In some embodiments, the isolated antibodies of antigen-binding portions thereof comprise a heavy chain variable region comprising a CDRH1 comprising SEQ ID NOs: 12, 14, 67, 68, 69, 70, 102, 103, 104, 123, 124, 125, or 126; a CDRH2 comprising SEQ ID NOs: 15, 17, 18, 19, 71, 72, 73, 74, 105, 106, 107, 108, 119, 120, 121, 122, 127, 128, 129, or 130; and a CDRH3 comprising SEQ ID NOs: 20, 22, 23, 75, 76, 109, 110, or 131; and a light chain variable region comprising a CDRL1 comprising SEQ ID NOs: 24, 26, 27, 77, 78, 79, 111, 112, 113, or 114; a CDRL2 comprising SEQ ID NOs: 28, 30, 80, 81, 82, 115, 116, or 117; and a CDRL3 comprising SEQ ID NOs: 31, 33, 83, or 118. In some embodiments, the isolated antibodies or antigen-binding portions thereof comprise a heavy chain variable region comprising a CDRH1 comprising SEQ ID NO: 12 or 14; a CDRH2 comprising SEQ ID NOs: 15, 17, 18, or 19; and a CDRH3 comprising SEQ ID NOs: 20, 22, or 23; and a light chain variable region comprising a CDRL1 comprising SEQ ID NOs: 24, 26, or 27; a CDRL2 comprising SEQ ID NO: 28 or 30; and a CDRL3 comprising SEQ ID NO: 31 or 33. In some embodiments, the isolated antibodies or antigen-binding portions thereof comprise a heavy chain variable region comprising a CDRH1 comprising SEQ ID NOs: 13, 84, 85, 86, 87, or 88; a CDRH2 comprising SEQ ID NOs: 16, 89, 90, 91, or 92; and a CDRH3 comprising SEQ ID NOs: 21, 93, or 94; and a light chain variable region comprising a CDRL1 comprising SEQ ID NOs: 25, 95, 96, or 97; a CDRL2 comprising SEQ ID NOs: 29, 98, 99, or 100; and a CDRL3 comprising SEQ ID NOs: 32 or 101. In some embodiments, the isolated antibodies or antigen-binding portions thereof comprise a heavy chain variable region comprising a CDRH1 comprising SEQ ID NO: 13; a CDRH2 comprising SEQ ID NO: 16; and a CDRH3 comprising SEQ ID NO: 21; and a light chain variable region comprising a CDRL1 comprising SEQ ID NO: 25; a CDRL2 comprising SEQ ID NO: 29; and a CDRL3 comprising SEQ ID NO: 32. In some embodiments, the antibody or antigen binding portion is a scFv protein listed in any of Table 5-Table 12.

In some embodiments, the isolated antibodies or antigen binding portions thereof comprise a heavy chain variable region comprising an amino acid sequence that is at least 90% identical to SEQ ID NO:1; and a light chain variable region comprising an amino acid sequence that is at least 90% identical to SEQ ID NO: 7 or 11. In some embodiments, the isolated antibodies or antigen-binding portions thereof comprise a heavy chain variable region comprising an amino acid sequence that is at least 90% identical to SEQ ID NO:2; and a light chain variable region comprising an amino acid sequence that is at least 90% identical to SEQ ID NO:8. In some embodiments, the isolated antibodies or antigen-binding portions thereof comprise a heavy chain variable region comprising an amino acid sequence that is at least 90% identical to SEQ ID NO:3; and a light chain variable region comprising an amino acid sequence that is at least 90% identical to SEQ ID NO: 9 or 10. In some embodiments, the isolated antibodies or antigen-binding portions thereof comprise a heavy chain variable region comprising an amino acid sequence that is at least 90% identical to SEQ ID NO: 4 or 5; and a light chain variable region comprising an amino acid sequence that is at least 90% identical to SEQ ID NO:7. In some embodiments, the isolated antibodies or antigen-binding portions thereof comprise a heavy chain variable region comprising an amino acid sequence that is at least 90% identical to SEQ ID NO:6; and a light chain variable region comprising an amino acid sequence that is at least 90% identical to SEQ ID NO:11.

Also provided herein are chimeric antigen receptors (CARs) comprising an extracellular target-binding domain comprising an antibody or antigen binding portion thereof described herein; a transmembrane domain; and a signaling domain. In some embodiments, the antibody or antigen binding portion thereof is a single chain antibody fragment, a single chain Fv (scFv), a single chain Fab, a single chain Fab′, a single domain antibody fragment, a single domain multispecific antibody, an intrabody, a nanobody, or a single chain immunokine. In some embodiments, the antibody or antigen binding portion thereof is a scFv comprising an amino acid sequence that is at least 90% identical to SEQ ID NO:53. In some embodiments, the hinge and transmembrane domains comprise CD8a and CD28 hinge and transmembrane domains. In some embodiments, the signaling domain comprises a 4-1BB signaling domain, a CD28 signaling domain, an OX-40 signaling domain, and/or a CD3ζ signaling domain. In some embodiments, the CARs provided herein further comprise a leader sequence and/or a hinge domain.

In some embodiments, the CAR comprises an amino acid sequence that is at least 90% identical to SEQ ID NOs: 51, 60, 62, 64, or 66. In some embodiments, the CAR is encoded by a nucleic acid molecule comprising a nucleotide sequence that is at least 65% identical (for example, at least 70%, 75%, 80%, 85%, 90%, 91%, 92%, 93%, 94%, 95%, 96%, 97%, 98%, 99%, or 100% identical) to SEQ ID NOs: 50, 59, 61, 63, or 65. In some embodiments, the extracellular target-binding domain further comprises one or more additional antigen-binding domains. In some embodiments, the one or more additional antigen-binding domains specifically bind to CD19, CD20, CD22, CD79a, CD79b, or any combination thereof.

Also provided herein are recombinant nucleic acid molecules encoding an antibody or antigen binding portion thereof or a CAR as described herein. In some embodiments, the recombinant nucleic acid molecules are synthetic sequences designed for expression in a host cell. In some embodiments, the antibody or antigen binding portion is a scFv protein listed in any of Table 5-Table 12. In some embodiments, the nucleic acid molecule comprises a nucleotide sequence that is at least 65% identical (for example, at least 70%, 75%, 80%, 85%, 90%, 91%, 92%, 93%, 94%, 95%, 96%, 97%, 98%, 99%, or 100% identical) to SEQ ID NOs: 50, 59, 61, 63, or 65. In some embodiments, the nucleic acid molecule encodes an amino acid sequence that is at least 90% identical to SEQ ID NOs: 51, 60, 62, 64, or 66.

Also provided herein are DNA constructs comprising any of the recombinant nucleic acid molecules described herein operably linked to a promoter that drives expression in a host cell.

Also provided herein are vectors comprising any of the recombinant nucleic acid molecules or DNA constructs described herein.

Also provided herein are host cells comprising any of the recombinant nucleic acid molecules, DNA constructs, or vectors provided herein. In some embodiments, the host cells are bacterial cells. In some embodiments, the host cells are eukaryotic cells. In some embodiments, the host cells are immune effector cells. In some embodiments, the immune effector cells are T cells.

Also provided herein are compositions comprising an antibody or antigen binding portion thereof or a CAR as described herein; and a pharmaceutically acceptable carrier.

Also provided herein are methods of detecting a presence of ROR1 in a biological sample, the method comprising: contacting said biological sample with an isolated antibody or antigen binding portion thereof as described herein, and detecting an amount of binding of the isolated antibody or antigen binding portion thereof as a determination of the presence of ROR1 in the biological sample. In some embodiments, the biological sample comprises cancer cells. In some embodiments, the biological sample comprises a tumor sample of a tumor from a subject.

Also provided herein are methods of treating a cancer of a subject, the method comprising administering to the subject, e.g., the patient, a pharmaceutically effective amount of a composition described herein. In some embodiments, the cancer is a ROR1-expressing cancer. In some embodiments, the cancer comprises at least one of lymphoma, leukemia, or a solid tumor cancer. In some embodiments, the lymphoma is follicular lymphoma, mantle cell lymphoma, diffuse large B-cell lymphoma, or marginal zone lymphoma. In some embodiments, the leukemia is chronic lymphocytic leukemia. In some embodiments, the solid tumor cancer is breast cancer, ovarian cancer, colon cancer, lung cancer, skin cancer, pancreatic cancer, testicular cancer, bladder cancer, uterus cancer, prostate cancer, or adrenal cancer.

In some embodiments of the methods of treating a subject with cancer provided herein, the isolated antibody or antigen binding portion thereof is conjugated to a therapeutic agent. In some embodiments, the therapeutic agent is at least one of a cytotoxic agent, a chemotherapeutic agent, or an immunosuppressive agent. In some embodiments, the therapeutic agent is a moiety that specifically binds to an immune cell. In some embodiments, the immune cell is a T cell. In some embodiments, the immune cell is a natural killer cell.

In some embodiments of the methods of treating a subject with cancer provided herein, the methods further comprise administering a second form of cancer therapy to the subject. In some embodiments, the second form of cancer therapy comprises a cytotoxic agent, a chemotherapeutic agent, an immunosuppressive agent, or radiation therapy.

Also provided herein are methods of imaging a tumor in a subject with a ROR1-expressing cancer, the method comprising administering to the subject an isolated antibody or antigen binding portion thereof as described herein conjugated to an imaging label, and detecting the imaging label in the subject to obtain an image of the tumor.

Also provided herein are methods of monitoring response of a subject to a cancer therapy, wherein the subject has a ROR1-expressing cancer, the methods comprising (a) administering to the subject, at a first time point before the subject receives the cancer therapy, an isolated antibody or antigen binding portion thereof as described herein conjugated to an imaging label; (b) detecting the imaging label in the subject to obtain a first image of a tumor in the subject; (c) administering to the subject, at a second time point after the subject receives the cancer therapy, an isolated antibody or antigen binding portion thereof as described herein conjugated to an imaging label; (d) detecting the imaging label in the subject to obtain a second image of the tumor; and (e) comparing the first image to the second image to determine whether a change in tumor size has occurred. In some embodiments, steps (c) to (e) are repeated at a third time point after the subject receives the cancer therapy. In some embodiments, the imaging label comprises a radioisotope, a bioluminescent label, a chemiluminescent label, or a paramagnetic compound.

Also provided herein are methods of assessing responsiveness of a subject to a treatment with a ROR1 targeted therapy, wherein the subject has a cancer, the methods comprising measuring an amount of ROR1 in a tumor sample from the subject; determining, based on the amount of ROR1, if the cancer is characterized as having a high level of ROR1 expression; and indicating that the subject is more likely to respond to the treatment if the cancer is characterized as having a high level of ROR1 expression or that the subject is less likely to respond to the treatment if the cancer is characterized as having a low level of ROR1 expression wherein at least one of (i) the ROR1 targeted therapy comprises administration of a composition described herein, or (ii) the amount of ROR1 in the tumor sample is measured using an isolated antibody or antigen binding portion thereof as described herein.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 shows expression of ROR1 in normal tissues and in lymphomas, according to aspects of this disclosure. Relative expression of ROR1 mRNA was assessed via quantitative PCR in 22 normal adult human tissues (top panel) and in primary lymphoma tumor cells (CLL: chronic lymphocytic leukemia; MCL: mantle cell lymphoma; DLBCL: diffuse large B-cell lymphoma; FL: follicular lymphoma) and peripheral blood B cells (labelled “B”) and T cells (labelled “T”) from normal donors (middle panel). ROR1 protein expression was assessed using flow cytometry in primary lymphoma tumor cells and normal B cells (bottom panel). Dashed line: ROR1 antibody; solid line: isotype control antibody.

FIG. 2 shows generation of ROR1-expressing L cells and testing of antibodies by ELISA, according to aspects of this disclosure. Cells from a mouse fibroblast cell line, L cells, were transduced with human ROR1 cDNA. Expression of human ROR1 was assessed by commercial anti-ROR1 antibody from Biolegend (Dashed histogram in top panel) and compared with isotype control antibody (Gray histogram in top panel). ROR1-L cells, but not parental L cells, expressed high levels of ROR1. The binding of eight anti-ROR1 antibodies (A89, A97, A129, A130, A140, A142, A148, and A183) to ROR1 was tested against ROR1-L and L cells by ELISA (bottom panel). “Sed”: secondary antibody only (negative control).

FIG. 3 shows specificity of anti-ROR1 antibodies, according to aspects of this disclosure. The binding of eight anti-ROR1 antibodies (A89, A97, A129, A130, A140, A142, A148, and A183) was tested against ROR1-L and parental L cells, as well as ROR1-positive lymphoma cell lines SP53 (MCL), Jeko-1 (MCL), and CA46 (Burkitt lymphoma) using flow cytometry. Dashed line: anti-ROR1 antibodies; Gray line: isotype control antibody; Black line: unstained.

FIG. 4 shows specificity of anti-ROR1 antibodies, according to aspects of this disclosure. The binding of anti-ROR1 antibody A89 was assessed by flow cytometry against peripheral blood T cells, B cells, and monocytes derived from a normal donor. Dashed line: A89 antibody; Gray line: isotype control antibody; Black line: unstained. Similar results were obtained for the remaining seven anti-ROR1 antibodies (A97, A129, A130, A140, A142, A148, and A183).

FIG. 5 shows generation of anti-ROR1 CAR-T cells, according to aspects of this disclosure. The top panel shows a schematic design of an anti-ROR1 CAR derived from antibody clone A129. The bottom panel shows expression of the anti-ROR1 CAR 72 hours after lentiviral transduction via flow-cytometry detection of a FITC-conjugated ROR1 extracellular domain (ECD) protein. Transduced T cells showed positive staining with ROR1 ECD protein, indicating the CAR was expressed on the cell surface and binds to the native ROR1 ECD.

FIGS. 6A-6B show that anti-ROR1 CAR-T cells are cytotoxic against B-cell lymphoma cell lines, according to aspects of this disclosure. Anti-ROR1 CAR T cells (derived from clone A129) were co-cultured with ROR1-transduced L and parental L cells labeled with CELLTRACE™ Violet overnight at an effector:target ratio (E:T) of 1:1, and the percentage of dead L or ROR1-transduced L cells was determined by staining for dead cells (top panel). FIG. 6A shows that anti-ROR1 CAR T cells induced significant lysis of ROR1-L cells but not L cells. Anti-ROR1 CAR T cells or untransduced T cells were co-cultured with ROR1+ B-cell lymphoma lines (CA46, Jeko-1, Sp53) or ROR1 negative NK92 cell line labeled with CELLTRACE™ Violet at E:T1:T2 ratio of 1:1:1 for 4 days. The percentage of dead cells was determined by dead cell staining on days 1, 2, 3 and 4. Percent specific lysis was assessed by counting live tumor cells using TRUCOUNT™ beads and data were normalized to untransduced T cell controls, as shown in FIG. 6B.

FIGS. 7A-7C show that multiple anti-ROR1 CAR constructs exhibit cytotoxicity against B-cell lymphoma cell lines, according to aspects of this disclosure. Anti-ROR1 CAR-T cells with different hinge and transmembrane domains (CD8a or CD28) and costimulatory domains (CD28, 4-1BB, or OX40) (see Table 13) were co-cultured with green fluorescent protein (GFP)-transduced, ROR1-expressing B-cell lymphoma cell lines, CA46 (FIG. 7A), Jeko-1 (FIG. 7B), and SP53 (FIG. 7C) at an Effector:Target (E:T) ratio of 1:1. The cytotoxic activity of the CAR-T cells was assessed by determining change in GFP-positive cells (green area confluence) by Incucyte live cell imaging serially over time. Anti-ROR1 CAR-T cells induced significant lysis of ROR1+ B-cell lymphoma lines compared to tumor alone and untransduced T cell (UnT) groups (p<0.05). Anti-CD19 CAR-T cells (CD19) were used as positive control. The experiments were performed in triplicate wells.

FIGS. 8A-8C show that anti-ROR1 CAR-T cells (see Table 13) exhibit long-term cytotoxicity against B-cell lymphoma cell lines, according to aspects of this disclosure. Anti-ROR1 CAR-T cells with different hinge and transmembrane domains (CD8a or CD28) and costimulatory domains (CD28, 4-1BB, or OX40) were co-cultured with green fluorescent protein (GFP)-transduced, ROR1-expressing B-cell lymphoma cell lines, CA46 (FIG. 8A), Jeko-I (FIG. 8B), and SP53 (FIG. 8C) at an Effector:Target (E:T) ratio of 1:1. Fresh batches of tumor cells were added to the wells every 2 days and the cytotoxic activity of the CAR-T cells was assessed by determining change in GFP-positive cells (green area confluence) by INCUCYTE® live cell imaging serially over time for 15 days (15d). Anti-ROR1 CAR-T cells exhibited significant cytotoxic activity against ROR1+ B-cell lymphoma cell lines long-term for over 2 weeks compared to tumor alone and untransduced T cell (UnT) groups (p<0.05). Anti-CD19 CAR-T cells (CD19) were used as positive control. The experiments were performed in triplicate wells.

FIGS. 9A-9B show that multiple anti-ROR1 CAR constructs exhibit antitumor effects against Jeko-1 B-cell lymphoma cell line in vivo, according to aspects of this disclosure. Luciferase-transduced ROR1+Jeko-1 mantle cell lymphoma tumor cells were injected via tail vein into NSG mice (0.3×10⁶ cells/mouse) on day −7. On day 0, 5×10⁶ anti-ROR1 CAR-T cells with different hinge and transmembrane domains (CD8a or CD28) and costimulatory domains (CD28, 4-1BB, or OX40) (see Table 13) were injected via tail vein. FIG. 9. Tumor burden was determined by bioluminescent imaging at the indicated time points. Anti-ROR1 CAR-T cells induced rapid elimination of tumor cells compared to tumor alone and untransduced T cell groups by day 7. Anti-CD19 CAR-T cells were used as positive control.

### DETAILED DESCRIPTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

### I. Terminology

The following definitions are provided to assist the reader. Unless otherwise defined, all terms of art, notations, and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be construed as representing a substantial difference over the definition of the term as generally understood in the art.

Articles “a” and “an” are used herein to refer to one or to more than one (i.e., at least one) of the grammatical object of the article. By way of example, “an element” means at least one element and can include more than one element.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of and “consisting of those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The terms “about” and “approximately” as used herein shall generally mean an acceptable degree of error for the quantity measured given the nature or precision of the measurements. Exemplary degrees of error are within 20% (%); preferably, within 10%; and more preferably, within 5% of a given value or range of values. Any reference to “about X” or “approximately X” specifically indicates at least the values X, 0.95X, 0.96X, 0.97X, 0.98X, 0.99X, 1.01X, 1.02X, 1.03X, 1.04X, and 1.05X. Thus, expressions “about X” or “approximately X” are intended to teach and provide written support for a claim limitation of, for example, “0.98X.” Alternatively, in biological systems, the terms “about” and “approximately” may mean values that are within an order of magnitude, preferably within 5-fold, and more preferably within 2-fold of a given value. Numerical quantities given herein are approximate unless stated otherwise, meaning that the term “about” or “approximately” can be inferred when not expressly stated. When “about” is applied to the beginning of a numerical range, it applies to both ends of the range.

As used throughout, the term “nucleic acid” or “nucleotide” refers to deoxyribonucleic acids (DNA) or ribonucleic acids (RNA) and polymers thereof in either single- or double-stranded form. It is understood that when an RNA is described, its corresponding cDNA is also described, wherein uridine is represented as thymidine. Unless specifically limited, the term encompasses nucleic acids containing known analogues of natural nucleotides that have similar properties as the reference nucleic acid and are metabolized in a manner similar to naturally occurring nucleotides. A nucleic acid sequence can comprise combinations of deoxyribonucleic acids and ribonucleic acids. Such deoxyribonucleic acids and ribonucleic acids include both naturally occurring molecules and synthetic analogues. The polynucleotides of the invention also encompass all forms of sequences including, but not limited to, single-stranded forms, double-stranded forms, hairpins, stem-and-loop structures, and the like.

Unless otherwise indicated, a particular nucleic acid sequence also implicitly encompasses conservatively modified variants thereof, alleles, orthologs, SNPs, and complementary sequences as well as the sequence explicitly indicated.

The term “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 88%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

“Polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bonds.

The amino acids in the polypeptides described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Zhang et al. “Protein engineering with unnatural amino acids,” Curr. Opin. Struct. Biol. 23(4): 581-587 (2013); Xie et la. “Adding amino acids to the genetic repertoire,” 9(6): 548-54 (2005)); and all references cited therein. Beta and gamma amino acids are known in the art and are also contemplated herein as unnatural amino acids.

As used herein, a chemically modified amino acid refers to an amino acid whose side chain has been chemically modified. For example, a side chain can be modified to comprise a signaling moiety, such as a fluorophore or a radiolabel. A side chain can also be modified to comprise a new functional group, such as a thiol, carboxylic acid, or amino group. Post-translationally modified amino acids are also included in the definition of chemically modified amino acids.

### II. Introduction

Provided in this disclosure are antibodies and antigen binding portions thereof that specifically bind receptor tyrosine kinase like orphan receptor 1 (ROR1), various compositions of such antibodies or antigen binding portions thereof, recombinant nucleic acids encoding the antibodies and antigen binding portions thereof, and associated methods of use. ROR1 is a transmembrane receptor for Wnt5a within the receptor tyrosine kinase (RTK) family. After binding to Wnt5a, ROR1 recruits and activates Rho GTPases and enhances the migration, proliferation, and survival of cells (see Yu et al., 2016, The Journal of Clinical Investigation 126(2):585-598). ROR1 is expressed at high level in embryonic tissues but absent in virtually all normal adult tissues (Fukuda et al., 2008, Proc. Nat. Acad. Sci. USA 105(8):3047-3052). However, ROR1 is expressed at a high level in cells of various cancers where corresponding normal cells do not express ROR1, referred to herein as ROR1-expressing cancers, including hematological cancers such as chronic lymphocytic leukemia (CLL), mantle cell lymphoma (MCL), marginal zone lymphoma, and several solid tumors including breast cancer, colon cancer and melanoma (Zhang et al., 2012, The American Journal of Pathology 181(6):1903-1910). A small percentage of cancer cells with characteristics of undifferentiated leukemia cells or cancer stem cells were found to overexpress ROR1, suggesting that ROR1 may play a role in tumor initiation and/or therapy resistance (Zhang et al., 2014, Proc. Nat. Acad Sci. USA 111(48):17266-17271).

Early studies found ROR1-deficient mice demonstrated a variety of phenotypic defects with skeletal and urogenital system abnormalities and postnatal growth retardation (Lyashenko et al., 2010, Developmental Dynamics 239(8):2266-2277). In cancer cells, siRNA knock down of ROR1 impaired the growth of breast cancer cells and sensitized the chemo-resistant lung tumor cell to treatment with tyrosine kinase inhibitors (erlotinib), indicating that immunotherapy strategies targeting ROR1 can be an effective treatment for cancers besides B-cell malignancies (Zhang et al., 2012, PLoS ONE 7(3):e31127; Wang et al., 2019, Oncology Letters 18(3):2977-2984). Indeed, ROR1 protein was found to be significantly overexpressed in 40% of breast cancers, 54% of ovarian cancers, 57% of colon cancers, 77% of lung cancers, 90% of lymphomas and CLL, 89% of skin cancers, 83% of pancreatic cancers, 73% of testicular cancers, 43% of bladder cancers, 96% of uterus cancers, 90% of prostate cancers, and 83% of adrenal cancers (Barna et al., 2011, Hematological Oncology 29(1):17-21). Moreover, the expression of ROR1 protein correlated with highly progressive disease with increased cell migratory capacity and an undifferentiated phenotype of cancer cells (Hojjat-Farsangi et al., 2014, Seminars in Cancer Biology 29:21-31). In lung cancer, ROR1 and EGFR can be physically attached to each other, and ROR1 is required to sustain EGFR survival signaling in lung adenocarcinoma (Yamaguchi et al., 2012, Cancer Cell 21(3):348-361).

As demonstrated in the Examples herein, the provided antibodies specifically bind to ROR1 in isogenic cell lines and in B-cell lymphoma cell lines, but not in normal peripheral blood B cells, T cells, or monocytes. Also provided herein are various molecules comprising said antibodies, including chimeric antigen receptors (CARs), antibody-drug conjugates, and bispecific antibodies (e.g., T-cell engagers), as well as recombinant nucleic acids encoding such molecules, host cells comprising such molecules, and associated methods of use. Further provided herein are diagnostic and therapeutic methods using the antibodies, antigen binding portions thereof, recombinant nucleic acids encoding the antibodies, and/or host cells comprising the antibodies. The inventors have demonstrated that lentiviral transduction of a CAR comprising a ROR1 antibody into healthy donor T cells redirected their specificity against B-cell lymphoma cell lines, and that such anti-ROR1 CAR T cells were highly cytotoxic to B-cell lymphoma cell lines that express ROR1, but not to control cell lines. The antibodies and associated methods provided herein represent a novel approach for treating and diagnosing patients with refractory B-cell malignancies and other cancers that express ROR1.

### III. Antibodies

In one aspect, the present disclosure provides antibodies and antigen binding portions thereof that bind specifically to ROR1. As used herein, the term antibody encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called alpha, delta, epsilon, gamma, and mu, respectively. As used herein, the term antibody also encompasses an antibody fragment, for example, an antigen binding fragment. Antigen binding fragments comprise at least one antigen binding domain. One example of an antigen binding domain is an antigen binding domain formed by a VH-VL dimer. Antibodies and antigen binding fragments can be described by the antigen to which they specifically bind.

The term variable is used herein to describe certain portions of the antibody domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a j-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies. The constant domains are not involved directly in binding an antibody to an antigen, but exhibit various effector functions, such as participation of the antibody in antibody-dependent cellular toxicity. Each VH and VL generally comprises three CDRs and four FRs, arranged in the following order (from N-terminus to C-terminus): FR1-CDR1-FR2-CDR2-FR3-CDR3-FR4. The CDRs are involved in antigen binding, and confer antigen specificity and binding affinity to the antibody. (See Kabat et al. (1991) Sequences of Proteins of Immunological Interest 5th ed., Public Health Service, National Institutes of Health, Bethesda, MD.) CDR sequences on the heavy chain (VH) may be designated as CDRH1, 2, 3, while CDR sequences on the light chain (VL) may be designated as CDRL1, 2, 3.

As used herein, the terms binds specifically to, specific for, binds selectively to and selective for ROR1 or an epitope on a ROR1 protein mean binding that is measurably different from a non-specific or non-selective interaction. Specific binding can be measured, for example, by determining binding of a molecule compared to binding of a control molecule. Specific binding can also be determined by competition with a control molecule that is similar to the target, such as an excess of non-labeled target. In that case, specific binding is indicated if the binding of the labeled target to a probe is competitively inhibited by the excess non-labeled target.

The ROR1 antibodies and antigen binding portions thereof are polypeptides. The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids. The term “protein” as used herein refers to either a polypeptide or a dimer (i.e., two) or multimer (i.e., three or more) of single chain polypeptides. The single chain polypeptides of a protein may be joined by a covalent bond, e.g., a disulfide bond, or non-covalent interactions. The terms “portion” and “fragment” are used interchangeably herein to refer to parts of a polypeptide, nucleic acid, or other molecular construct.

Provided herein are antibodies or antigen binding portions thereof that bind specifically to ROR1. ROR1-specific antibodies were identified and tested as described in the Examples below. In some embodiments, heavy chain variable region sequences and light chain variable region sequences encompassed by this disclosure are set forth in Table 1. In certain embodiments, heavy chain CDR sequences encompassed by this disclosure are set forth in Table 2. In certain embodiments, light chain CDR sequences encompassed by this disclosure are set forth in Table 3. In Table 1, the CDR sequences in the variable domains are indicated by bold and underlined text. In some embodiments, the antibody or antigen binding portion is a scFv protein listed in any of Table 5-Table 12.

In some embodiments, the antibody or antigen binding fragment thereof has a heavy chain variable region that includes (i) a CDRH1 comprising SEQ ID NOs: 12, 13, 14, 67, 68, 69, 70, 84, 85, 86, 87, 88, 102, 103, 104, 123, 124, 125, or 126; (ii) a CDRH2 comprising SEQ ID NOs: 15, 16, 17, 18, 19, 71, 72, 73, 74, 89, 90, 91, 92, 105, 106, 107, 108, 119, 120, 121, 122, 127, 128, 129, or 130; and (iii) a CDRH3 comprising SEQ ID NOs: 20, 21, 22, 23, 75, 76, 93, 94, 109, 110, or 131; and a light chain variable region that includes (i) a CDRL1 comprising SEQ ID NOs: 24, 25, 26, 27, 77, 78, 79, 95, 96, 97, 111, 112, 113, or 114; (ii) a CDRL2 comprising SEQ ID NOs: 28, 29, 30, 80, 81, 82, 98, 99, 100, 115, 116, or 117; and (iii) a CDRL3 comprising SEQ ID NOs: 31, 32, 33, 83, 101, or 118.

In some embodiments, the antibody or antigen binding fragment thereof has a heavy chain variable region that includes (i) a CDRH1 comprising SEQ ID NOs: 12, 14, 67, 68, 69, 70, 102, 103, 104, 123, 124, 125, or 126; (ii) a CDRH2 comprising SEQ ID NOs: 15, 17, 18, 19, 71, 72, 73, 74, 105, 106, 107, 108, 119, 120, 121, 122, 127, 128, 129, or 130; and (iii) a CDRH3 comprising SEQ ID NOs: 20, 22, 23, 75, 76, 109, 110, or 131; and a light chain variable region that includes (i) a CDRL1 comprising SEQ ID NOs: 24, 26, 27, 77, 78, 79, 111, 112, 113, or 114; (ii) a CDRL2 comprising SEQ ID NOs: 28, 30, 80, 81, 82, 115, 116, or 117; and (iii) a CDRL3 comprising SEQ ID NOs: 31, 33, 83, or 118.

In some embodiments, the antibody or antigen binding fragment thereof has a heavy chain variable region that includes (i) a CDRH1 comprising SEQ ID NOs: 12 or 14; (ii) a CDRH2 comprising SEQ ID NO: 15, 17, 18, or 19; and (iii) a CDRH3 comprising SEQ ID NO: 20, 22, or 23; and a light chain variable region that includes (i) a CDRL1 comprising SEQ ID NO: 24, 26, or 27; (ii) a CDRL2 comprising SEQ ID NO: 28 or 30; and (iii) a CDRL3 comprising SEQ ID NO: 31 or 33.

In some embodiments, the antibody or antigen binding fragment thereof has a heavy chain variable region that includes (i) a CDRH1 comprising SEQ ID NOs: 12, 67, 68, 69, or 70; (ii) a CDRH2 comprising SEQ ID NOs: 15, 71, 72, 73, or 74; and (iii) a CDRH3 comprising SEQ ID NOs: 20, 75, or 76; and a light chain variable region that includes (i) a CDRL1 comprising SEQ ID NOs: 24, 78, or 79; (ii) a CDRL2 comprising SEQ ID NOs: 28, 80, 81, or 82; and (iii) a CDRL3 comprising SEQ ID NOs: 31 or 83.

In some embodiments, the antibody or antigen binding fragment thereof has a heavy chain variable region that includes (i) a CDRH1 comprising SEQ ID NOs: 13, 84, 85, 86, 87, or 88; (ii) a CDRH2 comprising SEQ ID NOs: 16, 89, 90, 91, or 92; and (iii) a CDRH3 comprising SEQ ID NOs: 21, 93, or 94; and a light chain variable region that includes (i) a CDRL1 comprising SEQ ID NOs: 25, 95, 96, or 97; (ii) a CDRL2 comprising SEQ ID NOs: 29, 98, 99, or 100; and (iii) a CDRL3 comprising SEQ ID NOs: 32 or 101.

In some embodiments, the antibody or antigen binding fragment thereof has a heavy chain variable region that includes (i) a CDRH1 comprising SEQ ID NO:13; (ii) a CDRH2 comprising SEQ ID NO:16; and (iii) a CDRH3 comprising SEQ ID NO:21; and a light chain variable region that includes (i) a CDRL1 comprising SEQ ID NO: 25; (ii) a CDRL2 comprising SEQ ID NO:29; and (iii) a CDRL3 comprising SEQ ID NO:32.

In some embodiments, the antibody or antigen binding fragment thereof has a heavy chain variable region that includes (i) a CDRH1 comprising SEQ ID NOs: 12, 67, 102, 103, or 104; (ii) a CDRH2 comprising SEQ ID NOs: 17, 105, 106, 107, or 108; and (iii) a CDRH3 comprising SEQ ID NOs: 22, 109, or 110; and a light chain variable region that includes (i) a CDRL1 comprising SEQ ID NOs: 26, 111, or 112; (ii) a CDRL2 comprising SEQ ID NOs: 28, 80, 81, or 82; and (iii) a CDRL3 comprising SEQ ID NOs: 31 or 83.

In some embodiments, the antibody or antigen binding fragment thereof has a heavy chain variable region that includes (i) a CDRH1 comprising SEQ ID NOs: 12, 67, 102, 103, or 104; (ii) a CDRH2 comprising SEQ ID NOs: 17, 105, 106, 107, or 108; and (iii) a CDRH3 comprising SEQ ID NOs: 22, 109, or 110; and a light chain variable region that includes (i) a CDRL1 comprising SEQ ID NOs: 27, 113, or 114; (ii) a CDRL2 comprising SEQ ID NOs: 30, 115, 116, or 117; and (iii) a CDRL3 comprising SEQ ID NOs: 33 or 118.

In some embodiments, the antibody or antigen binding fragment thereof has a heavy chain variable region that includes (i) a CDRH1 comprising SEQ ID NOs: 12, 67, 102, 103, or 104; (ii) a CDRH2 comprising SEQ ID NOs: 18, 119, 120, 121, or 122; and (iii) a CDRH3 comprising SEQ ID NOs: 20, 75, or 76; and a light chain variable region that includes (i) a CDRL1 comprising SEQ ID NOs: 24, 77, or 78; (ii) a CDRL2 comprising SEQ ID NOs: 28, 80, 81, or 82; and (iii) a CDRL3 comprising SEQ ID NOs: 31 or 83.

In some embodiments, the antibody or antigen binding fragment thereof has a heavy chain variable region that includes (i) a CDRH1 comprising SEQ ID NOs: 14, 123, 124, 125, 126; (ii) a CDRH2 comprising SEQ ID NOs: 19, 127, 128, 129, or 130; and (iii) a CDRH3 comprising SEQ ID NOs: 20, 23, or 131; and a light chain variable region that includes (i) a CDRL1 comprising SEQ ID NOs: 24, 77, or 78; (ii) a CDRL2 comprising SEQ ID NOs: 28, 80, 81, or 82; and (iii) a CDRL3 comprising SEQ ID NOs: 31 or 83.

In some embodiments, the antibody or antigen binding fragment thereof has a heavy chain variable region that includes (i) a CDRH1 comprising SEQ ID NOs: 12, 67, 102, 103, or 104; (ii) a CDRH2 comprising SEQ ID NOs: 15, 71, 72, 73, or 74; and (iii) a CDRH3 comprising SEQ ID NOs: 20, 75, or 76; and a light chain variable region that includes (i) a CDRL1 comprising SEQ ID NOs: 26, 111, or 112; (ii) a CDRL2 comprising SEQ ID NOs: 28, 80, 81, or 82; and (iii) a CDRL3 comprising SEQ ID NOs: 31 or 83.

In some embodiments, the antibody or antigen binding fragment thereof has a heavy chain variable region that includes (i) a CDRH1 comprising SEQ ID NOs: 12, 67, 68, 69, or 70; (ii) a CDRH2 comprising SEQ ID NOs: 15, 71, 72, 73, or 74; and (iii) a CDRH3 comprising SEQ ID NOs: 20, 75, or 76; and a light chain variable region that includes (i) a CDRL1 comprising SEQ ID NOs: 26, 111, or 112; (ii) a CDRL2 comprising SEQ ID NOs: 28, 80, 81, or 82; and (iii) a CDRL3 comprising SEQ ID NOs: 31 or 83.

In some embodiments, the antibody or antigen binding fragment thereof has a heavy chain variable region comprising an amino acid sequence that is at least 90% identical (for example, at least 90%, 91%, 92%, 93%, 94%, 95%, 96%, 97%, 98% or 99% identical) to SEQ ID NO:1; and a light chain variable region that includes an amino acid sequence that is at least 90% identical (for example, at least 90%, 91%, 92%, 93%, 94%, 95%, 96%, 97%, 98% or 99% identical) to SEQ ID NOs: 7 or 11.

In some embodiments, the antibody or antigen binding fragment thereof has a heavy chain variable region comprising an amino acid sequence that is at least 90% identical (for example, at least 90%, 91%, 92%, 93%, 94%, 95%, 96%, 97%, 98% or 99% identical) to SEQ ID NO:2; and a light chain variable region that includes an amino acid sequence that is at least 90% identical (for example, at least 90%, 91%, 92%, 93%, 94%, 95%, 96%, 97%, 98% or 99% identical) to SEQ ID NO:8.

In some embodiments, the antibody or antigen binding fragment thereof has a heavy chain variable region comprising an amino acid sequence that is at least 90% identical (for example, at least 90%, 91%, 92%, 93%, 94%, 95%, 96%, 97%, 98% or 99% identical) to SEQ ID NO:3; and a light chain variable region that includes an amino acid sequence that is at least 90% identical (for example, at least 90%, 91%, 92%, 93%, 94%, 95%, 96%, 97%, 98% or 99% identical) to SEQ ID NOs: 9 or 10.

In some embodiments, the antibody or antigen binding fragment thereof has a heavy chain variable region comprising an amino acid sequence that is at least 90% identical (for example, at least 90%, 91%, 92%, 93%, 94%, 95%, 96%, 97%, 98% or 99% identical) to SEQ ID NOs: 4 or 5; and a light chain variable region that includes an amino acid sequence that is at least 90% identical (for example, at least 90%, 91%, 92%, 93%, 94%, 95%, 96%, 97%, 98% or 99% identical) to SEQ ID NO:7.

In some embodiments, the antibody or antigen binding fragment thereof has a heavy chain variable region comprising an amino acid sequence that is at least 90% identical (for example, at least 90%, 91%, 92%, 93%, 94%, 95%, 96%, 97%, 98% or 99% identical) to SEQ ID NO:6; and a light chain variable region that includes an amino acid sequence that is at least 90% identical (for example, at least 90%, 91%, 92%, 93%, 94%, 95%, 96%, 97%, 98% or 99% identical) to SEQ ID NO:11.

In some embodiments, the antibody or antigen binding fragment thereof has a heavy chain variable region comprising a CDRH3 comprising any one of SEQ ID NOs: 20-23.

In some embodiments, the antibody or antigen binding fragment thereof has a heavy chain variable region comprising any of the CDR-H1, CDR-H2, or CDR-H3 sequences listed in Tables 5-12, below. In some embodiments, the antibody or antigen binding fragment thereof has a light chain variable region comprising any of the CDR-L1, CDR-L2, or CDR-L3 sequences listed in Tables 5-12, below.

In some embodiments, the antibody or antigen binding fragment thereof includes a heavy chain variable region comprising an amino acid sequence that is at least 90% identical (for example, at least 90%, 91%, 92%, 93%, 94%, 95%, 96%, 97%, 98% or 99% identical) to any of SEQ ID NOs: 1-6.

The disclosure also provides an antibody or antigen binding portion thereof that specifically binds to ROR1, wherein the antibody or antigen binding portion thereof comprises a heavy chain variable region comprising an amino acid sequence that is at least 90% identical (for example, at least 90%, 91%, 92%, 93%, 94%, 95%, 96%, 97%, 98% or 99% identical) to any of SEQ ID NOs: 1-6 and a light chain variable region comprising an amino acid sequence that is at least 90% identical (for example, at least 90%, 91%, 92%, 93%, 94%, 95%, 96%, 97%, 98% or 99% identical) to any of SEQ ID NOs: 7-11. Table 1 provides the sequences for SEQ ID Nos: 1-11.

In some embodiments, the antibody or antigen binding portion thereof comprises a heavy chain variable region or a light chain variable region comprising an amino acid sequence that is at least 90% identical (for example, at least 90%, 91%, 92%, 93%, 94%, 95%, 96%, 97%, 98% or 99% identical) to any of the sequences in Table 2.

In each case, where a specific amino acid sequence is recited, embodiments comprising a sequence having at least 90% (e.g., 90%, 91%, 92%, 93%, 94%, 95%, 96%, 97%, 98%, or 99%) identity to the recited sequence are also provided.

TABLE 1
Antibody VH and VL amino acid sequences of selected clones.

| Anti- | | |
|---|---|---|
| body | | |
| ID | VH sequence | VL sequence |
| A89 | SEQ ID NO: 1 | SEQ ID NO: 7 |
| | DVQLQESGPGLVKPSQSLSLTCSVSGYSITSGYYWSWIRQFP | DVLMTQTPLSLPVSLGDQASISCRSSQSIVHSNGNTYLEWY |
| | GNKLEWMGYISNDGSNKYKPSLKNRISITRDTSKNQFFLKL | LQKPGQSPKLLIYKVSNRFSGVPDRFSGSGSGTDFTLKISRVE |
| | KSVTTEDTATYYCARFPLINYDPWLAYWGQGTLVTVSA | AEDLGVYYCFQGSHVPWTFGGGTKLEIN |
| A97 | SEQ ID NO: 2 | SEQ ID NO: 8 |
| | EVQLQQSGPELEKPGASVKISCKASGYSFIGYSMNWVRQSD | DIVLTQSPATLSVTPGDSVSLSCRASQSITNNLHWYQQKSHE |
| | GKSLEWIGNIDPYYGGTSYNLKFKGKATLTVDKSSSTAYM | SPRLLIKSASQSISGIPSRFSGSGSGTDFTLSINSVETEDFGMY |
| | QLKSLTSEDSAVYYCARLGRGYFDYWGQGTTLTVSS | FCQQSNSWPHTFGGGTKLEIK |
| A129 | SEQ ID NO: 3 | SEQ ID NO: 9 |
| | DVQLQESGPGLVKPSQSLSLTCSVTGYSITSGYYWNWIRQF | DVLMTQAPLSLPVSLGDQASISCRSSQNVVHRNGNTYLEW |
| | PGNKLEWMGYIRNDGSNNYNPSLKNRISITRDTSKNQFFLK | YLQKPGQSPKLLIYKVSNRFSGVPDRFSGSGSGTDFTLKISR |
| | LNSVTTEDTATYYCARFPLINYDPWFAYWGQGTLVTVSA | VEAEDLGVYYCFQGSHVPWTFGGGTKLEIK |
| A130 | SEQ ID NO: 3 | SEQ ID NO: 10 |
| | DVQLQESGPGLVKPSQSLSLTCSVTGYSITSGYYWNWIRQF | ETTVTQSPASLSVATGEKVTIRCITSTDIDDDMNWYQQKPG |
| | PGNKLEWMGYIRNDGSNNYNPSLKNRISITRDTSKNQFFLK | EPPKLLISEANTLRPGVPSRFSSSGYGTDFVFTIENTLSEDVA |
| | LNSVTTEDTATYYCARFPLINYDPWFAYWGQGTLVTVSA | DYYCLQSYNMPLTFGAGTTLELK |
| A140 | SEQ ID NO: 4 | SEQ ID NO: 7 |
| | DVQLQESGPGLVKPSQSLSLTCSVSGYSITSGYYWNWIRQF | DVLMTQTPLSLPVSLGDQASISCRSSQSIVHSNGNTYLEWY |
| | PGNKLEWMGYISNDGNNKYKPSLKNRISITRDTSKNQFFLK | LQKPGQSPKLLIYKVSNRFSGVPDRFSGSGSGTDFTLKISRVE |
| | LNSVTAEDTATYYCARFPLINYDPWLAYWGQGTLVTVSA | AEDLGVYYCFQGSHVPWTFGGGTKLEIN |
| A142 | SEQ ID NO: 5 | SEQ ID NO: 7 |
| | EVQLQQSGPDL VKPGASVKISCKASGYSFTGYYMHWVKQS | DVLMTQTPLSLPVSLGDQASISCRSSQSIVHSNGNTYLEWY |
| | HGKSLEWIGRVYPNNGGTSYNQKFKGKAILTVDKSSTTAY | LQKPGQSPKLLIYKVSNRFSGVPDRFSGSGSGTDFTLKISRVE |
| | MELRSLTSEDSAVYYCALIHYYGFYSMDYWGQGTSVTVSS | AEDLGVYYCFQGSHVPWTFGGGTKLEIN |
| A148 | SEQ ID NO: 6 | SEQ ID NO: 11 |
| | DVQLQESGPGLVKPSQSLSLTCSVSGYSITSGYYWNWIRQF | DVLMTQTPLSLPVSLGDQASISCRSSQNVVHRNGNTYLEW |
| | PGNKLEWMGYISNDGSNKYKPSLKNRISITRDTSKNQFFLK | YLQKPGQSPKLLIYKVSNRFSGVPDRFSGSGSGTDFTLKISR |
| | LNSVTTEDTATYYCARFPLINYDPWLAYWGQGTL VTVSA | VEAEDLGVYYCFQGSHVPWTFGGGTKLEIK |
| A183 | SEQ ID NO: 1 | SEQ ID NO: 11 |
| | DVQLQESGPGLVKPSQSLSLTCSVSGYSITSGYYWSWIRQFP | DVLMTQTPLSLPVSLGDQASISCRSSQNVVHRNGNTYLEW |
| | GNKLEWMGYISNDGSNKYKPSLKNRISITRDTSKNQFFLKL | YLQKPGQSPKLLIYKVSNRFSGVPDRFSGSGSGTDFTLKISR |
| | KSVTTEDTATYYCARFPLINYDPWLAYWGQGTLVTVSA | VEAEDLGVYYCFQGSHVPWTFGGGTKLEIK |

TABLE 2
Heavy chain variable domain
(VH) CDR amino acid sequences.

| Antibody | VH | | | |
|---|---|---|---|---|
| ID | sequence | CDR1 | CDR2 | CDR3 |
| A89 | SEQ ID | GYSITSGYY | ISNDGSN | ARFPLIN |
| | NO: 1 | (SEQ ID | (SEQ ID | YDPWLAY |
| | | NO: 12) | NO: 15) | (SEQ ID |
| | | | | NO: 20) |
| A97 | SEQ ID | GYSFIGYS | IDPYYGGT | ARLGRGY |
| | NO: 2 | (SEQ ID | (SEQ ID | FDY |
| | | NO: 13) | NO: 16) | (SEQ ID |
| | | | | NO: 21) |
| A129 | SEQ ID | GYSITSGYY | IRNDGSN | ARFPLIN |
| | NO: 3 | (SEQ ID | (SEQ ID | YDPWFAY |
| | | NO: 12) | NO: 17) | (SEQ ID |
| | | | | NO: 22) |
| A130 | SEQ ID | GYSITSGYY | IRNDGSN | ARFPLIN |
| | NO: 3 | (SEQ ID | (SEQ ID | YDPWFAY |
| | | NO: 12) | NO: 17) | (SEQ ID |
| | | | | NO: 22) |
| A140 | SEQ ID | GYSITSGYY | ISNDGNN | ARFPLIN |
| | NO: 4 | (SEQ ID | (SEQ ID | YDPWLAY |
| | | NO: 12) | NO: 18) | (SEQ ID |
| | | | | NO: 20) |
| A142 | SEQ ID | GYSFTGYY | VYPNNGGT | ALIHYYG |
| | NO: 5 | (SEQ ID | (SEQ ID | FYSMDY |
| | | NO: 14) | NO: 19) | (SEQ ID |
| | | | | NO: 23) |
| A148 | SEQ ID | GYSITSGYY | ISNDGSN | ARFPLIN |
| | NO: 6 | (SEQ ID | (SEQ ID | YDPWLAY |
| | | NO: 12) | NO: 15) | (SEQ ID |
| | | | | NO: 20) |
| A183 | SEQ ID | GYSITSGYY | ISNDGSN | ARFPLIN |
| | NO: 1 | (SEQ ID | (SEQ ID | YDPWLAY |
| | | NO: 12) | NO: 15) | (SEQ ID |
| | | | | NO: 20) |

TABLE 3
Light chain variable domain
(VL) CDR amino acid sequences.

| Antibody | VH | | | |
|---|---|---|---|---|
| ID | sequence | CDR1 | CDR2 | CDR3 |
| A89 | SEQ ID | QSIVHS | KVS | FQGSHVPWT |
| | NO: 7 | NGNTY | (SEQ ID | (SEQ ID |
| | | (SEQ ID | NO: 28) | NO: 31) |
| | | NO: 24) | | |
| A97 | SEQ ID | QSITNN | SAS | QQSNSWPHT |
| | NO: 8 | (SEQ ID | (SEQ ID | (SEQ ID |
| | | NO: 25) | NO: 29) | NO: 32) |
| A129 | SEQ ID | QNVVHR | KVS | FQGSHVPWT |
| | NO: 9 | NGNTY | (SEQ ID | (SEQ ID |
| | | (SEQ ID | NO: 28) | NO: 31) |
| | | NO: 26) | | |
| A130 | SEQ ID | TDIDDD | EAN | LQSYNMPLT |
| | NO: 10 | (SEQ ID | (SEQ ID | (SEQ ID |
| | | NO: 27) | NO: 30) | NO: 33) |
| A140 | SEQ ID | QSIVHS | KVS | FQGSHVPWT |
| | NO: 7 | NGNTY | (SEQ ID | (SEQ ID |
| | | (SEQ ID | NO: 28) | NO: 31) |
| | | NO: 24) | | |
| A142 | SEQ ID | QSIVHS | KVS | FQGSHVPWT |
| | NO: 7 | NGNTY | (SEQ ID | (SEQ ID |
| | | (SEQ ID | NO: 28) | NO: 31) |
| | | NO: 24) | | |
| A148 | SEQ ID | QNVVHR | KVS | FQGSHVPWT |
| | NO: 11 | NGNTY | (SEQ ID | (SEQ ID |
| | | (SEQ ID | NO: 28) | NO: 31) |
| | | NO: 26) | | |
| A183 | SEQ ID | QNVVHR | KVS | FQGSHVPWT |
| | NO: 11 | NGNTY | (SEQ ID | (SEQ ID |
| | | (SEQ ID | NO: 28) | NO: 31) |
| | | NO: 26) | | |

The amino acid residue sequences provided herein are set forth in single-letter amino acid code which can be used interchangeably with three-letter amino acid code. An amino acid refers to any monomer unit that can be incorporated into a peptide, polypeptide, or protein. The twenty natural or genetically encoded alpha-amino acids are as follows: alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). The structures of these twenty natural amino acids are shown in, e.g., Stryer et al., Biochemistry, 5^{th} ed., Freeman and Company (2002). The term amino acid also includes unnatural amino acids, modified amino acids (e.g., having modified side chains and/or backbones), and amino acid analogs.

The terms identical or percent identity, in the context of two or more nucleic acids or polypeptide sequences, refer to two or more sequences or subsequences that are the same or have a specified percentage of nucleotides or amino acid residues that are the same (e.g., at least 60%, at least 65%, at least 70%, at least 75%, at least 80%, at least 85%, at least 90%, or at least 95% or greater identity over a specified region), when compared and aligned for maximum correspondence over a comparison window, or designated region as measured using one of the following sequence comparison algorithms or by manual alignment and visual inspection.

Identity or similarity with respect to a sequence is defined as the percentage of amino acid residues in the candidate sequence that are identical (i.e., same residue) with the starting amino acid residues, after aligning the sequences and introducing gaps, if necessary, to achieve the maximum percent sequence identity. Methods of alignment of sequences for comparison are well known in the art, e.g., visual alignment or using publicly available software using known algorithms to achieve maximal alignment. Optimal alignment of sequences for comparison can be conducted, for example, by the local homology algorithm of Smith and Waterman (Adv. Appl. Math. 2:482, 1970), by the homology alignment algorithm of Needleman and Wunsch (J. Mol. Biol. 48:443, 1970), by the search for similarity method of Pearson and Lipman (Proc. Natl. Acad. Sci. USA 85:2444, 1988), by computerized implementations of these algorithms (e.g., GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)). Other publicly available software useful for alignment analysis include BLAST programs, ALIGN, ALIGN-2 (Genentech, South San Francisco, Calif.), and Megalign (DNASTAR).

As with all peptides, polypeptides, and proteins, including fragments thereof, it is understood that additional modifications in the amino acid sequence of the ROR1-specific antibodies or antigen binding fragments thereof described herein, for example, in the heavy chain variable region and/or light chain variable region, can occur that do not alter the nature or function of the antibodies or antigen binding fragments thereof. Such modifications include conservative amino acids substitutions, such that each recited sequence optionally contains one or more conservative amino acid substitutions. The list provided below identifies groups that contain amino acids that are conservative substitutions for one another; these groups are exemplary as other conservative substitutions are known to those of skill in the art:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

By way of example, when an aspartic acid at a specific residue is mentioned, also contemplated is a conservative substitution at the residue, for example, glutamic acid. Non-conservative substitutions, for example, substituting a proline with glycine or substituting a lysine with an asparagine, are also contemplated.

In some instances, the affinity of ROR1-specific antibodies or antigen binding fragments thereof may be optimized through mutations to increase or decrease affinity as desired based on one or more of the known characteristics of the binding interaction with ROR1, the structure of either or both of the antibodies or fragments thereof, or the ROR1 protein. In some instances, the mutations permit facile elution of purified antibodies or fragments thereof under desirable elution conditions during isolation and purification.

Methods of generating and screening for antibodies and antigen binding fragments thereof as provided in this disclosure are described in the Examples and are well-known in the art. Methods of further modifying antibodies for enhanced properties (e.g., enhanced affinity, chimerization, humanization) as well as generating antigen binding fragments, as described herein, are also well-known in the art.

In some embodiments, the heavy chain variable region and/or the light chain variable region of the monoclonal antibody has an identical sequence to the heavy chain variable region and/or the light chain variable region of the antibody produced by the methods described herein and in the Examples below. In some embodiments, the heavy chain variable region and/or the light chain variable region of the monoclonal antibody comprises one or more modifications, e.g., amino acid substitutions, deletions, or insertions.

The heavy chain variable region sequence and/or light chain variable region sequence of an antibody described herein can be engineered to comprise one or more variations in the heavy chain variable region sequence and/or light chain variable region sequence. In some embodiments, the engineered variation(s) improves the binding affinity of the antibody for ROR1. In some embodiments, the engineered variation(s) improves the cross-reactivity of the antibody for a second antigen.

In some embodiments, the engineered variation is a variation in one or more CDRs, e.g., an amino acid substitution in a heavy chain CDR and/or a light chain CDR as described herein. In some embodiments, the engineered variation is a variation in one or more framework regions, e.g., an amino acid substitution in a heavy chain framework region and/or a light chain framework region. In some embodiments, the engineered variation is a reversion of a region of the heavy chain and/or light chain sequence to the inferred naïve sequence. Methods for determining an inferred naïve immunoglobulin sequence are described in the art. See, e.g., Magnani et al., PLoS Negl Trop Dis, 2017, 11:e0005655, doi:10.1371/journal.pntd.0005655.

In some embodiments, affinity maturation is used to engineer further mutations that enhance the binding affinity of the antibody for ROR1 or enhance the cross-reactivity of the antibody for a second antigen. Methods for performing affinity maturation are known in the art. See, e.g., Renaut et al., Methods Mol Biol, 2012, 907:451-461.

The present disclosure also encompasses antibodies or fragments thereof that bind to the same epitope of ROR1 as the antibodies disclosed herein. Such antibodies can be identified using routine techniques known in the art, including, for example, competitive binding assays.

The term epitope, as used herein, means a component of an antigen capable of specific binding to an antibody or antigen binding fragment thereof. Such components optionally comprise one or more contiguous amino acid residues and/or one or more non-contiguous amino acid residues. Epitopes frequently consist of surface-accessible amino acid residues and/or sugar side chains and can have specific three-dimensional structural characteristics, as well as specific charge characteristics. Conformational and non-conformational epitopes are distinguished in that the binding to the former but not the latter is lost in the presence of denaturing solvents. An epitope can comprise amino acid residues that are directly involved in the binding, and other amino acid residues, which are not directly involved in the binding. The epitope to which an antigen binding protein binds can be determined using known techniques for epitope determination such as, for example, testing for antigen binding protein binding to antigen variants with different point mutations.

The present disclosure also provides chimeric antibodies. The term chimeric antibody refers to an antibody in which a component of the heavy and/or light chain is derived from a particular source or species, while the remainder of the heavy and/or light chain is derived from a different source or species.

A human antibody is one that possesses an amino acid sequence corresponding to that of an antibody produced by a human or a human cell, or derived from a non-human source that utilizes a human antibody repertoire or human antibody-encoding sequences (e.g., obtained from human sources, genetically modified non-human sources or designed de novo). Human antibodies specifically exclude humanized antibodies.

Humanized forms of non-human antibodies are chimeric antibodies that contain minimal sequence derived from the non-human antibody. A humanized antibody is generally a human immunoglobulin (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies can also comprise residues that are not found in either the recipient antibody or the donor antibody. Such modifications can be made to further refine antibody function. (See Jones et al. (1986) Nature, 321:522-525; Riechmann et al. (1988) Nature, 332:323-329; and Presta, (1992) Curr Op Struct Biol., 2:593-596).

In some embodiments, the antibody or antigen binding fragment thereof provided herein can include a heavy (H) chain variable domain sequence (abbreviated herein as VH), and a light (L) chain variable domain sequence (abbreviated herein as VL). In some embodiments, an antibody molecule comprises or consists of a heavy chain and a light chain (referred to as a half antibody). In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv, for example), single variable domain antibodies, diabodies (Dab) (bivalent and bispecific), and chimeric (e.g., humanized) antibodies, which may be produced by the modification of whole antibodies or synthesized de novo using recombinant DNA technologies. These functional antibody fragments retain the ability to bind specifically to their respective antigen. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The preparation of antibody molecules can be monoclonal or polyclonal. An antibody molecule can also be a human, humanized, CDR-grafted, or an in vitro generated antibody. The antibody can have a heavy chain constant region chosen from, e.g., IgG1, IgG2, IgG3, or IgG4. The antibody can also have a light chain chosen from either kappa or lambda light chains.

As used herein, the term monoclonal antibody refers to an antibody from a population of substantially homogeneous antibodies. A population of substantially homogeneous antibodies comprises antibodies that are the same or substantially similar and that bind the same epitope(s), except for variants that can normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of yeast clones, phage clones, bacterial clones, mammalian cell clones, hybridoma clones, or other recombinant DNA clones. The selected antibody can be further altered, for example, to improve affinity for the target, for example, by affinity maturation, to humanize the antibody, to improve its production in cell culture, and/or to reduce its immunogenicity in a subject.

Antigen binding fragments of an antibody molecule are well known in the art, and include, for example, (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv) (see e.g., Bird et al. (1988) Science 242:423-426; Huston et al. (1988) Proc. Natl. Acad. Sci. USA 85:5879-5883); (viii) a single domain antibody. These antibody fragments are obtained using conventional techniques known to those skilled in the art, and the fragments are screened for utility in the same manner as are intact antibodies.

In certain embodiments, antibodies and antibody compositions as provided herein are distinguishable from naturally occurring antibodies and compositions in one or more respects. Such distinguishable antibodies and compositions may be referred to as “synthetic,” or may be identified by the proviso that the antibody or composition “is not naturally occurring” or affirmatively as “non-naturally occurring.” As used herein the terms “corresponding antibody,” and “corresponding to” describes the relationship between (1) an antibody characterized by six specific CDR sequences and produced by the methods described herein and in the Examples below and (2) a synthetic antibody comprising the same six CDR sequences. Synthetic antibodies of this disclosure may differ in structure from naturally occurring antibodies with the same CDRs. That is, synthetic antibodies identified by specified CDRs may be structurally different from antibodies comprising the specified CDRs that are produced by the methods described herein and in the Examples below. Possible differences for synthetic antibodies include variable region sequences that differ from corresponding naturally occurring antibodies, different light chain sequences (i.e., lambda type instead of kappa type or vice versa), different isotypes, different allotypes, and different constant domain variants. These differences are discussed in more detail below.

In one approach, an antibody heavy chain comprises the CDRs of a clone described herein with the proviso that the antibody heavy chain does not comprise the heavy chain variable region sequence associated with the clone described herein. For illustration, in one embodiment an antibody that comprises the CDRs of antibody A89 does not have a heavy chain variable region that comprises SEQ ID NO:1. In another approach, an antibody light chain comprises the CDRs of a clone described herein with the proviso that the antibody light chain does not comprise the light chain variable region sequence associated with the clone described herein. For illustration, in one embodiment an antibody that comprises the CDRs of antibody A89 does not have a light chain variable region that comprises SEQ ID NO:7. In some instances, both the heavy chain and the light chain variable region of an antibody have an amino acid sequence other than the sequence disclosed herein.

In some embodiments the synthetic antibody with specified CDRs is an isotype other than the isotype(s) found associated with the antibodies produced by the methods described herein and in the Examples below. In some embodiments the antibody disclosed herein is an isotype other than IgG1. In some embodiments the antibody disclosed herein is an isotype other than IgG2. In some embodiments the antibody disclosed herein is an isotype other than IgG3. In some embodiments the antibody disclosed herein is an isotype other than IgG4. In some embodiments the antibody disclosed herein is an isotype other than IgM. In some embodiments the antibody disclosed herein is an isotype other than IgA. In some embodiments the synthetic antibody comprises lambda type light chains. In some embodiments the synthetic antibody comprises kappa type light chains.

In some embodiments, the monoclonal antibody comprises a heavy chain variable region sequence and a light chain variable region sequence that are derived from an immunoglobulin producing human B cell, and further comprises a kappa or lambda light chain constant region. In some embodiments, the light chain constant region (kappa or lambda) is from the same type of light chain (i.e., kappa or lambda) as the light chain variable region that was derived from the immunoglobulin producing human B cell; as a non-limiting example, if an IgE-producing human B cell comprises a kappa light chain, then the monoclonal antibody that is produced can comprise the light chain variable region from the IgE-producing B cell and further comprises a kappa light chain constant region.

In some embodiments, the monoclonal antibody comprises a heavy chain variable region sequence and a light chain variable region sequence that are derived from an immunoglobulin-producing human B cell, and further comprises a heavy chain constant region having an IgG isotype (e.g., IgG4), an IgA isotype (e.g., IgA1), an IgM isotype, an IgD isotype, or that is derived from an IgG, IgA, IgM, or IgD isotype (e.g., is a modified IgG4 constant region). It will be appreciated by a person of ordinary skill in the art that the different heavy chain isotypes (IgA, IgD, IgE, IgG, and IgM) have different effector functions that are mediated by the heavy chain constant region, and that for certain uses it may be desirable to have an antibody that has the effector function of a particular isotype (e.g., IgG).

In some embodiments, the monoclonal antibody comprises a native (i.e., wild-type) human IgG, IgA, IgM, or IgD constant region. In some embodiments, the monoclonal antibody comprises a native human IgG1 constant region, a native human IgG2 constant region, a native human IgG3 constant region, a native human IgG4 constant region, a native human IgA1 constant region, a native human IgA2 constant region, a native human IgM constant region, or a native human IgD constant region. In some embodiments, the monoclonal antibody comprises a heavy chain constant region that comprises one or more modifications. It will be appreciated by a person of ordinary skill in the art that modifications such as amino acid substitutions can be made at one or more residues within the heavy chain constant region that modulate effector function. In some embodiments, the modification reduces effector function, e.g., results in a reduced ability to induce certain biological functions upon binding to an Fc receptor expressed on an effector cell that mediates the effector function. In some embodiments, the modification (e.g., amino acid substitution) prevents in vivo Fab arm exchange, which can introduce undesirable effects and reduce the therapeutic efficacy of the antibody. See, e.g., Silva et al., J Biol Chem, 2015, 280:5462-5469.

In some embodiments, the monoclonal antibody comprises a native (i.e., wild-type) human IgM constant region, human IgD constant region, human IgG constant region that is derived from IgG1, IgG2, IgG3, or IgG4, or human IgA constant region that is derived from IgA1 or IgA2 and comprises one or more modifications that modulate effector function. In some embodiments the monoclonal antibody comprises a human IgM constant region, human IgD constant region, human IgG constant region that is derived from IgG1, IgG2, IgG3, or IgG4, or human IgA constant region that is derived from IgA1 or IgA2. In some embodiments, the monoclonal antibody comprises a native (i.e., wild-type) human IgM constant region, human IgD constant region, human IgG constant region that is derived from IgG1, IgG2, IgG3, or IgG4, or human IgA constant region that is derived from IgA1 or IgA2 and comprises one, two, three, four, five, six, seven, eight, nine, ten or more modifications (e.g., amino acid substitutions). In some embodiments the constant regions include variations (e.g., one, two, three, four, five, six, seven, eight, nine, ten or more amino acid substitutions) that reduce effector function.

In some embodiments the synthetic antibody with specified CDRs is an allotype other the allotype(s) found associated with the antibodies produced by the methods described herein and in the Examples below. The synthetic antibody may comprise an allotype selected from those listed in Table 4, below, which is different from an allotype of antibodies produced by the methods described herein and in the Examples below. In some embodiments, the synthetic antibody may comprise any individual allotype selected from those listed in Table 4, with the proviso that the allotype differs from the corresponding allotype of antibodies produced by the methods described herein and in the Examples below.

TABLE 4
Human immunoglobulin allotypes.

| | Heavy chains | | | | |
|---|---|---|---|---|---|
| Isotype/type | IgG1 | IgG2 | IgG3 | IgA | Light chains |
| Allotypes | G1m | G2m | G3m | A2m | Km |
| | 1(a) | 23(n) | 21(g1) | 1 | 1 |
| | 2(x) | | 28(g5) | 2 | 2 |
| | 3(f) | | 11(b0) | | 3 |
| | 17(z) | | 5(b1) | | |
| | | | 13 (b3) | | |
| | | | 14 (b4) | | |
| | | | 10 (b5) | | |
| | | | 15(s) | | |
| | | | 16(t) | | |
| | | | 6(c3) | | |
| | | | 24(c5) | | |
| | | | 26(u) | | |
| | | | 27 (v) | | |

NB: Alphabetical notation given within brackets. From: Jefferis and Marie-Paule Lefranc, 2009, “Human immunoglobulin allotypes: Possible implications for immunogenicity” mAbs 1(4): 332-338, incorporated herein by reference.

In some embodiments, a monoclonal antibody comprises CDR sequences, a heavy chain variable region, and/or a light chain variable region as described herein (e.g., as disclosed in Tables 1-3) and further comprises a heavy chain constant region and/or a light chain constant region that is heterologous to the antibody produced by the methods described herein and in the Examples below from which the CDR sequences and/or variable region sequences are derived. For example, in some embodiments, the monoclonal antibody comprises the CDR sequences and/or variable region sequences of an antibody produced by the methods described herein and in the Examples below, and further comprises a heavy chain constant region and a light chain constant region that is heterologous to the antibody produced by the methods described herein and in the Examples below (e.g., the heavy chain constant region and/or light chain constant region is a wild-type or modified IgG1, IgG2, IgG3, or IgG4 constant region), or the heavy chain constant region and/or light chain constant region comprises one or more modifications (e.g., amino acid substitutions) relative to the native constant region of the antibodies produced by the methods described herein and in the Examples below.

Synthetic antibodies of this disclosure may comprise variations in heavy chain constant regions to change the properties of the synthetic antibody relative to the corresponding naturally occurring antibody. Exemplary changes include mutations to modulate antibody effector function (e.g., complement-based effector function or FcγR-based effector function), alter half-like, modulate coengagement of antigen and FcγRs, introduce or remove glycosylation motifs (glyco-engineering). See Fonseca et al., 2018, “Boosting half-life and effector functions of therapeutic antibodies by Fc-engineering: An interaction-function review” Int J Biol Macromol. 19:306-311; Wang et al., 2018, “IgG Fe engineering to modulate antibody effector functions” Protein Cell 2018, 9(1):63-73; Schlothauer, 2016, “Novel human IgG1 and IgG4 Fc-engineered antibodies with completely abolished immune effector functions,” Protein Engineering, Design and Selection 29(10):457-466; Tam et al., 2017, “Functional, Biophysical, and Structural Characterization of Human IgG1 and IgG4 Fc Variants with Ablated Immune Functionality” Antibodies 6, 12, each incorporated herein by reference for all purposes.

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, rat, guinea, pig, human, camel, llama, fish, shark, goat, rabbit, and bovine. Single domain antibodies are described, for example, in International Application Publication No. WO 94/04678. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species (e.g., camel, llama, dromedary, alpaca and guanaco) or other species besides Camelidae.

In some embodiments, an antigen binding fragment can also be or can also comprise, e.g., a non-antibody, scaffold protein. These proteins are generally obtained through combinatorial chemistry-based adaptation of preexisting antigen-binding proteins. For example, the binding site of human transferrin for human transferrin receptor can be diversified using the system described herein to create a diverse library of transferrin variants, some of which have acquired affinity for different antigens. See, e.g., Ali et al. (1999) J. Biol. Chem. 274:24066-24073. The portion of human transferrin not involved with binding the receptor remains unchanged and serves as a scaffold, like framework regions of antibodies, to present the variant binding sites. The libraries are then screened, as an antibody library is screened, and in accordance with the methods described herein, against a target antigen of interest to identify those variants having optimal selectivity and affinity for the target antigen. See, e.g., Hey et al. (2005) TRENDS Biotechnol 23(10):514-522.

One of skill in the art would appreciate that the scaffold portion of the non-antibody scaffold protein can include, e.g., all or part of the Z domain of S. aureus protein A, human transferrin, human tenth fibronectin type III domain, kunitz domain of a human trypsin inhibitor, human CTLA-4, an ankyrin repeat protein, a human lipocalin (e.g., anticalins, such as those described in, e.g., International Application Publication No. WO2015/104406), human crystallin, human ubiquitin, or a trypsin inhibitor from E. elaterium.

Synthetic antibody compositions of this disclosure may differ from naturally occurring compositions in at least one or more of the following respects: (i) composition comprises antibodies that are purified, i.e., separated from tissue or cellular material with which they are associated in the human body, and optionally in a manufactured excipient or medium; and/or (ii) antibody compositions of this disclosure contain a single species of antibody (are monoclonal) such that all antibodies in the composition have the same structure and specificity.

Any of the ROR1-specific antibodies or antigen binding fragments thereof described herein can be modified with covalent and/or non-covalent modifications. Such modifications can be introduced into the antibodies or antigen binding fragments by, e.g., reacting targeted amino acid residues of the polypeptide with an organic derivatizing agent that is capable of reacting with selected side chains or terminal residues. Suitable sites for modification can be chosen using any of a variety of criteria including, e.g., structural analysis or amino acid sequence analysis of the antibodies or fragments. Recombinant techniques can be used to modify antibodies or antigen binding fragments thereof. For example, amino acids found to not contribute to either the activity or the binding specificity or affinity of the antibody can be deleted without a loss in the respective activity. Insertions, deletions, substitutions, or other selected modifications of particular regions or specific amino acids residues, provided the activity of the fragment is not significantly altered or impaired compared to the non-modified antibody, or antigen binding fragment thereof can be made. Such methods are readily apparent to a skilled practitioner in the art and can include site specific mutagenesis of the nucleic acid encoding the antibody or fragment thereof. (Zoller et al., Nucl. Acids Res. 10:6487-500 (1982)). In some instances, the ROR1-specific antibodies or antigen binding fragments may be labeled by a variety of means for use in diagnostic and/or pharmaceutical applications.

In some embodiments, the antibodies or antigen binding fragments thereof can be conjugated to a heterologous moiety. The heterologous moiety can be, e.g., a heterologous polypeptide, a therapeutic agent (e.g., a toxin or a drug), or a detectable label such as, but not limited to, a radioactive label, an enzymatic label, a fluorescent label, a heavy metal label, a luminescent label, or an affinity tag such as biotin or streptavidin. In some embodiments, the heterologous moiety is an antibody or antigen binding fragment thereof that specifically binds to a different target, and such a conjugated antibody is referred to as a bispecific antibody. Additional suitable heterologous polypeptides include, e.g., an antigenic tag (e.g., FLAG (DYKDDDDK) (SEQ ID NO:58), polyhistidine (6-His; HIHIHHH (SEQ ID NO:59)), hemagglutinin (HA; YPYDVPDYA (SEQ ID NO:60)), glutathione-S-transferase (GST), or maltose-binding protein (MBP)) for use in purifying the antibodies or fragments. Heterologous polypeptides also include polypeptides (e.g., enzymes) that are useful as diagnostic or detectable markers, for example, luciferase, a fluorescent protein (e.g., green fluorescent protein (GFP)), or chloramphenicol acetyl transferase (CAT). Suitable radioactive labels include, e.g., ³²P, ³³P, ¹⁴C, ¹²⁵I, ¹³¹I, ³⁵S, and ³H. Suitable fluorescent labels include, without limitation, fluorescein, fluorescein isothiocyanate (FITC), green fluorescent protein (GFP), DYLIGHT™ 488, phycoerythrin (PE), propidium iodide (PI), PerCP, PE-Alexa Fluor® 700, Cy5, allophycocyanin, and Cy7. Luminescent labels include, e.g., any of a variety of luminescent lanthanide (e.g., europium or terbium) chelates. For example, suitable europium chelates include the europium chelate of diethylene triamine pentaacetic acid (DTPA) or tetraazacyclododecane-1,4,7,10-tetraacetic acid (DOTA). Enzymatic labels include, e.g., alkaline phosphatase, CAT, luciferase, and horseradish peroxidase. Another labeling technique which may result in greater sensitivity consists of coupling the antibodies to low molecular weight haptens. These haptens can then be specifically altered by means of a second reaction. For example, it is common to use haptens such as biotin, which reacts with avidin, or dinitrophenol, pyridoxal, or fluorescein, which can react with specific antihapten antibodies. Additional acceptable heterologous moieties are described below in Section VII.

Two proteins (e.g., an antibody and a heterologous moiety) can be cross-linked using any of a number of known chemical cross linkers. Examples of such cross linkers are those that link two amino acid residues via a linkage that includes a “hindered” disulfide bond. In these linkages, a disulfide bond within the cross-linking unit is protected (by hindering groups on either side of the disulfide bond) from reduction by the action, for example, of reduced glutathione or the enzyme disulfide reductase. One suitable reagent, 4-succinimidyloxycarbonyl-α-methyl-α(2-pyridyldithio) toluene (SMPT), forms such a linkage between two proteins utilizing a terminal lysine on one of the proteins and a terminal cysteine on the other. Heterobifunctional reagents that cross-link by a different coupling moiety on each protein can also be used. Other useful cross-linkers include, without limitation, reagents which link two amino groups (e.g., N-5-azido-2-nitrobenzoyloxysuccinimide), two sulfhydryl groups (e.g., 1,4-bis-maleimidobutane), an amino group and a sulfhydryl group (e.g., m-maleimidobenzoyl-N-hydroxysuccinimide ester), an amino group and a carboxyl group (e.g., 4-[p-azidosalicylamido]butylamine), and an amino group and a guanidinium group that is present in the side chain of arginine (e.g., p-azidophenyl glyoxal monohydrate).

Techniques for conjugating a therapeutic moiety (e.g., any of those discussed in Section VII) to a ROR1-specific antibody or antigen binding fragment thereof as described herein are well known, see, for example, Arnon et al., Monoclonal Antibodies And Cancer Therapy, Reisfeld et al. (eds.), pp. 243-56 (1985); Hellstrom et al., Controlled Drug Delivery (2nd Ed.), Robinson et al. (eds.), pp. 623-53 (1987); Thorpe, Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy” In: Monoclonal Antibodies For Cancer Detection And Therapy, (Baldwin et al. eds.), pp. 303-316 (1985), and Thorpe et al., Immunol. Rev. 62:119-158 (1982). Alternatively, an antibody can be conjugated to a second antibody to form an antibody heteroconjugate (e.g., a bispecific antibody) as described in U.S. Pat. No. 4,676,980.

In some embodiments, a radioactive label can be directly conjugated to the amino acid backbone of the antibody. Alternatively, the radioactive label can be included as part of a larger molecule (e.g., ¹²⁵I in meta-[¹²⁵I]iodophenyl-N-hydroxysuccinimide ([¹²⁵I]mIPNHS), which binds to free amino groups to form meta-iodophenyl (mIP) derivatives of relevant proteins (see, e.g., Rogers et al. (1997) J Nucl Med 38:1221-1229) or chelate (e.g., to DOTA or DTPA), which is in turn bound to the protein backbone. Methods of conjugating the radioactive labels or larger molecules/chelates containing them to the antibodies or antigen binding fragments described herein are known in the art. Such methods involve incubating the proteins with the radioactive label under conditions (e.g., pH, salt concentration, and/or temperature) that facilitate binding of the radioactive label or chelate to the protein (see, e.g., U.S. Pat. No. 6,001,329).

Methods for conjugating a fluorescent label (sometimes referred to as a fluorophore) to a protein (e.g., an antibody) are known in the art of protein chemistry. For example, fluorophores can be conjugated to free amino groups (e.g., of lysines) or sulfhydryl groups (e.g., cysteines) of proteins using succinimidyl (NHS) ester or tetrafluorophenyl (TFP) ester moieties attached to the fluorophores. In some embodiments, the fluorophores can be conjugated to a heterobifunctional cross-linker moiety such as sulfo-SMCC. Suitable conjugation methods involve incubating an antibody protein or fragment thereof with the fluorophore under conditions that facilitate binding of the fluorophore to the protein. See, e.g., Welch and Redvanly (2003) Handbook of Radiopharmaceuticals: Radiochemistry and Applications, John Wiley and Sons.

In some embodiments, the antibodies or fragments can be modified, e.g., with a moiety that improves the stabilization and/or retention of the antibodies in circulation, e.g., in blood, serum, or other tissues. For example, the antibody or fragment can be PEGylated as described in, e.g., Lee et al. (1999) Bioconjug Chem 10(6): 973-8; Kinstler et al. (2002) Advanced Drug Deliveries Reviews 54:477-485; and Roberts et al. (2002) Advanced Drug Delivery Reviews 54:459-476, or HESylated (Fresenius Kabi, Germany) (see, e.g., Pavisid et al. (2010) Int J Pharm 387(1-2):110-119). The stabilization moiety can improve the stability, or retention of, the antibody (or fragment) by at least 1.5 (e.g., at least 2, 5, 10, 15, 20, 25, 30, 40, or 50 or more) fold.

In some embodiments, the antibodies or antigen-binding fragments thereof described herein can be glycosylated. In some embodiments, an antibody or antigen-binding fragment thereof described herein can be subjected to enzymatic or chemical treatment, or produced from a cell, such that the antibody or fragment has reduced or absent glycosylation. Methods for producing antibodies with reduced glycosylation are known in the art and described in, e.g., U.S. Pat. No. 6,933,368; Wright et al. (1991) EMBO J 10(10):2717-2723; and Co et al. (1993)Mol Immunol 30:1361.

### IV. Chimeric Antigen Receptors

Also provided herein are chimeric antigen receptors comprising any of the antibodies or antigen-binding fragments described above. Chimeric antigen receptors (CARs, also known as chimeric T cell receptors) are designed to be expressed in host effector cells, e.g., T cells or NK cells, and to induce an immune response against a specific target antigen and cells expressing that antigen. Adoptive T cell immunotherapy, in which a patient's own T lymphocytes are engineered to express CARs, has shown great promise in treating hematological malignancies. CARs can be engineered and used as described, for example, in Sadelain et al., 2013, Cancer Discov. 3:388-398. A CAR typically comprises an extracellular target-binding module, a transmembrane (TM) domain, and an intracellular signaling domain (ICD). The CAR domains can be joined via flexible hinge and/or spacer regions. The extracellular target-binding module generally comprises an antibody or antigen binding fragment thereof. In some instances, multiple binding specificities can be included in the extracellular target-binding module. For example, multiple antibodies or antigen binding fragments thereof that target different antigens can be included to produce bi-specific, tri-specific, or quad-specific CARs. TM domains are primarily considered a structural requirement, anchoring the CAR in the cell membrane, and are most commonly derived from molecules regulating T cell function, such as CD8 and CD28. The intracellular module typically consists of the T cell receptor CD3ζ chain and one or more costimulatory domains from either the Ig (CD28-like) or TNF receptor (TNFR) superfamilies. CARs containing either CD28 or 4-1BB costimulatory domains have been the most widely used, to date, and both of them have yielded dramatic responses in clinical trials. CAR domains are discussed in more detail below.

Provided herein are chimeric antigen receptors comprising: (a) an extracellular target-binding domain comprising a ROR1-specific antibody or antigen binding portion thereof, (b) a transmembrane domain; and (c) a signaling domain.

The extracellular target-binding module of a CAR may comprise an antibody or an antigen-binding fragment thereof that specifically binds a target antigen (e.g., ROR1). In certain embodiments, the extracellular target-binding domain can be a single-chain variable fragment derived from an antibody (scFv), a tandem scFv, a single-domain antibody fragment (V_{H}Hs or sdAbs), a single domain bispecific antibody (BsAbs), an intrabody, a nanobody, an immunokine in a single chain format, and Fab, Fab′, or (Fab′)₂ in a single chain format. In other embodiments, the extracellular target-binding domain can be an antibody moiety that comprises covalently bound multiple chains of variable fragments. In some embodiments, the extracellular target-binding domain comprises any of the antibodies or antigen-binding portions thereof described above. In some embodiments, the extracellular target-binding domain comprises a scFv comprising a heavy chain variable region comprising an amino acid sequence that is at least 90% identical (for example, at least 90%, 91%, 92%, 93%, 94%, 95%, 96%, 97%, 98% or 99% identical) to any of SEQ ID NOs:1-6 and a light chain variable region comprising an amino acid sequence that is at least 90% identical (for example, at least 90%, 91%, 92%, 93%, 94%, 95%, 96%, 97%, 98% or 99% identical) to any of SEQ ID NOs:7-11. In some embodiments, the scFv comprises a linker polypeptide between the heavy chain and light chain sequences (e.g., SEQ ID NO:54 or any of the other linkers described herein). In some embodiments, the CAR comprises a scFv comprising an amino acid sequence that is at least 90% identical to SEQ ID NO:53.

In some embodiments, the extracellular target-binding domains of the CARs provided herein further comprise one or more additional antigen-binding domains (i.e., in addition to the ROR1-specific antibody or antigen binding portion thereof, as described above). In some embodiments, the extracellular target-binding domain comprises one additional antigen-binding domain. CARs comprising such an extracellular target-binding domain can be referred to as bi-specific CARs. In some embodiments, the extracellular target-binding domain comprises two additional antigen-binding domains. CARs comprising such an extracellular target-binding domain can be referred to as tri-specific CARs. In some embodiments, the extracellular target-binding domain comprises three additional antigen-binding domain. CARs comprising such an extracellular target-binding domain can be referred to as quad-specific CARs. Each of the one or more additional antigen-binding domains may comprise an antibody or antigen binding portion thereof. In some embodiments, the one or more additional antigen-binding domains specifically bind to CD19, CD20, CD22, CD79a, CD79b, or any combination thereof.

The transmembrane domain of a CAR provided herein may be derived from either a natural or from a synthetic source. Where the source is natural, the domain may be derived from any membrane-bound or transmembrane protein. In some embodiments, the transmembrane domain is derived from (i.e., comprises at least the transmembrane region(s) of) the α, β, δ, γ, or ζ chain of the T-cell receptor, CD28, CD3ε, CD3ζ, CD45, CD4, CD5, CD8, CD9, CD16, CD22, CD30, CD33, CD37, CD64, CD80, CD86, CD134, CD137, or CD154. In some embodiments, a transmembrane domain can be chosen based on, for example, the nature of the various other proteins or trans-elements that bind the transmembrane domain or the cytokines induced by the transmembrane domain. In some embodiments, the transmembrane domain comprises a CD8α transmembrane domain (e.g., the amino acid sequence of SEQ ID NO:56). When a transmembrane domain is synthetic, it may comprise predominantly hydrophobic residues such as leucine and valine. In some embodiments, a triplet of phenylalanine, tryptophan, and valine may be found at each end of a synthetic transmembrane domain. In some embodiments, a short oligo- or polypeptide linker, having a length of, for example, between about 2 and about 10 (such as about any of 2, 3, 4, 5, 6, 7, 8, 9, or 10) amino acids in length may form the linkage between the transmembrane domain and the intracellular signaling domain of a CAR described herein. In some embodiments, the linker is a glycine-serine doublet.

The intracellular signaling domain of the CAR is responsible for activation of at least one of the normal effector functions of the immune cell in which the CAR has been placed in or is designed to be placed in. An effector function of a T cell may be, for example, cytolytic activity or helper activity, including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term “intracellular signaling sequence” is thus meant to include any truncated portion of the intracellular signaling domain sufficient to transduce the effector function signal.

Examples of intracellular signaling domains for use in the CARs provided herein include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or costimulatory signal is also required. Thus, T cell activation can be said to be mediated by two distinct classes of intracellular signaling sequence: those that initiate antigen-dependent primary activation through the TCR (primary signaling sequences) and those that act in an antigen-independent manner to provide a secondary or costimulatory signal (costimulatory signaling sequences).

Primary signaling sequences regulate primary activation of the TCR complex either in a stimulatory way, or in an inhibitory way. Primary signaling sequences that act in a stimulatory manner may contain signaling motifs which are known as immunoreceptor tyrosine-based activation motifs or ITAMs. In some embodiments, the CARs described herein comprise one or more ITAMs.

Examples of ITAM containing primary signaling sequences that are of particular use in the invention include those derived from TCRζ, FcRγ, FcRβ, CD3γ, CD3δ, CD3ε, CD3ζ, CD5, CD22, CD79a, CD79b, and CD66d. In some embodiments, an ITAM containing primary signaling sequence is derived from CD3ζ.

In some embodiments, the CAR comprises a primary signaling sequence derived from CD3ζ. For example, the intracellular signaling domain of the CAR can comprise the CD3ζ intracellular signaling sequence by itself or combined with any other desired intracellular signaling sequence(s) useful in the context of the CAR of the invention. In some embodiments, the intracellular signaling domain of a CAR provided herein comprises a CD3ζ primary intracellular signaling sequence (e.g., the amino acid sequence of SEQ ID NO:58) and a 4-1BB costimulatory signaling sequence (e.g., the amino acid sequence of SEQ ID NO:57).

The CARs provided herein may include additional elements, such a signal peptide to ensure proper export of the fusion protein to the cells surface, a leader sequence (e.g., SEQ ID NO:52), and a hinge domain (e.g., a CD8a hinge domain, e.g., SEQ ID NO:55) that imparts flexibility to the recognition region and allows strong binding to the targeted moiety. In some embodiments, a spacer domain may be present between any of the domains of the CAR. The spacer domain can be any polypeptide that functions to link two parts of the CAR. A spacer domain may comprise up to about 300 amino acids, including for example about 10 to about 100, or about 25 to about 50 amino acids. Methods of identifying and selecting suitable spacer domains are known.

In some embodiments, the CAR comprises at least one anti-ROR1 scFv domain as described in this disclosure; at least one hinge or transmembrane domain selected from the group consisting of CD8α and CD28 (e.g., as described in this disclosure, e.g., in any one of Table 5-Table 12); at least one costimulatory domain selected from the group consisting of CD28, 4-1BB, and OX40 (e.g., as described in this disclosure); and a CD3ζ signaling domain (e.g., as described in this disclosure). In some embodiments, a CAR provided herein comprises a sequence that is at least 90% identical (for example, at least 90%, 91%, 92%, 93%, 94%, 95%, 96%, 97%, 98% 99%, or 100% identical identical) to SEQ ID NOs: 51, 60, 62, 64, or 66. In some embodiments, a CAR provided herein is encoded by a nucleic acid molecule comprising a nucleotide sequence that is at least 65% identical (for example, at least 70%, 75%, 80%, 85%, 90%, 91%, 92%, 93%, 94%, 95%, 96%, 97%, 98%, 99%, or 100% identical) to SEQ ID NOs: 50, 59, 61, 63, or 65.

### V. Antibody Expression and Purification, Nucleic Acids, Vectors, and Cells

The ROR1 antibodies and antigen binding fragments thereof and molecules comprising such antibodies and antigen binding fragments thereof discussed above (e.g., CARs) may be produced by recombinant expression in a human or non-human cell. Synthetic antibody-producing cells include non-human cells expressing heavy chains, light chains, or both heavy and light chains; human cells that are not immune cells expressing heavy chains, light chains, or both heavy and light chains; and human B cells that produce heavy chains or light chains, but not both heavy and light chains. Synthetic antibodies of this disclosure may be heterologously expressed, in vitro or in vivo, in cells other than human B cells, such as non-human cells and human cells other than B cells, optionally other than immune cells, and optionally in cells other than cells in a B cell lineage.

The ROR1 antibodies and antigen binding fragments thereof and molecules comprising them described herein can be produced using a variety of techniques known in the art of molecular biology and protein chemistry. For example, a nucleic acid encoding the antibody or antigen binding fragment thereof can be inserted into an expression vector that contains transcriptional and translational regulatory sequences, which include, e.g., promoter sequences, ribosomal binding sites, transcriptional start and stop sequences, translational start and stop sequences, transcription terminator signals, polyadenylation signals, and enhancer or activator sequences. The regulatory sequences include a promoter and transcriptional start and stop sequences. In addition, the expression vector can include more than one replication system, such that it can be maintained in two different organisms, for example, in mammalian or insect cells for expression and in a prokaryotic host for cloning and amplification.

Several possible vector systems are available for the expression of cloned heavy chain and light chain polypeptides from nucleic acids in mammalian cells. One class of vectors relies upon the integration of the desired gene sequences into the host cell genome. Cells that have stably integrated DNA can be selected by simultaneously introducing drug resistance genes such as E. coli gpt (Mulligan and Berg (1981) Proc Natl Acad Sci USA 78:2072) or Tn5 neo (Southern and Berg (1982) Mol Appl Genet 1:327). The selectable marker gene can be either linked to the DNA gene sequences to be expressed or introduced into the same cell by co-transfection (Wigler et al. (1979) Cell 16:77). A second class of vectors utilizes DNA elements that confer autonomously replicating capabilities to an extrachromosomal plasmid. These vectors can be derived from animal viruses, such as bovine papillomavirus (Sarver et al. (1982) Proc Natl Acad Sci USA, 79:7147), CMV, polyoma virus (Deans et al. (1984) Proc Natl Acad Sci USA 81:1292), or SV40 virus (Lusky and Botchan (1981) Nature 293:79).

The expression vectors can be introduced into cells in a manner suitable for subsequent expression of the nucleic acid. The method of introduction is largely dictated by the targeted cell type, discussed below. Exemplary methods include CaPO₄ precipitation, liposome fusion, cationic liposomes, electroporation, nucleoporation, viral infection, dextran-mediated transfection, polybrene-mediated transfection, protoplast fusion, and direct microinjection.

Appropriate host cells for the expression of antibodies or antigen binding fragments thereof include yeast, bacteria, insect, plant, and mammalian cells. Of particular interest are bacteria such as E. coli, fungi such as Saccharomyces cerevisiae and Pichia pastoris, insect cells such as SF9, mammalian cell lines (e.g., human cell lines), as well as primary cell lines.

In some embodiments, an antibody or fragment thereof can be expressed in, and purified from, transgenic animals (e.g., transgenic mammals). For example, an antibody can be produced in transgenic non-human mammals (e.g., rodents) and isolated from milk as described in, e.g., Houdebine (2002) Curr Opin Biotechnol 13(6):625-629; van Kuik-Romeijn et al. (2000) Transgenic Res 9(2):155-159; and Pollock et al. (1999) J Immunol Methods 231(1-2):147-157.

The antibodies and fragments thereof can be produced from the cells by culturing a host cell transformed with the expression vector containing nucleic acid encoding the antibodies or fragments, under conditions, and for an amount of time, sufficient to allow expression of the proteins. Such conditions for protein expression vary with the choice of the expression vector and the host cell and are easily ascertained by one skilled in the art through routine experimentation. For example, antibodies expressed in E. coli can be refolded from inclusion bodies (see, e.g., Hou et al. (1998) Cytokine 10:319-30). Bacterial expression systems and methods for their use are known in the art (see Ausubel et al. (1988) Current Protocols in Molecular Biology, Wiley & Sons; and Green and Sambrook (2012) Molecular Cloning—A Laboratory Manual, 4th Ed., Cold Spring Harbor Laboratory Press, New York (2001)). The choice of codons, suitable expression vectors and suitable host cells vary depending on a number of factors, and may be easily optimized as needed. An antibody (or fragment thereof) described herein can be expressed in mammalian cells or in other expression systems including but not limited to yeast, baculovirus, and in vitro expression systems (see, e.g., Kaszubska et al. (2000) Protein Expression and Purification 18:213-220). Additional discussion of expression vectors for use in eukaryotic cells (e.g., for treating a subject with cancer), along with suitable delivery systems, is provided in Section VIII.A, below.

Also provided herein are nucleic acid molecules encoding a ROR1 antibody or antigen binding portion thereof that binds specifically to ROR1 as described in this disclosure.

In some embodiments, provided are nucleic acid molecules encoding a ROR1 antibody or antigen binding fragment thereof comprising a heavy chain variable comprising an amino acid sequence that is at least 90% identical (for example, at least 90%, 91%, 92%, 93%, 94%, 95%, 96%, 97%, 98% or 99% identical) to any of SEQ ID NOs: 1-6 and a light chain variable region comprising an amino acid sequence that is at least 90% identical to any one of SEQ ID NOs: 7-11. In some embodiments, provided are nucleic acid molecules encoding a ROR1 antibody or antigen binding fragment thereof comprising a heavy chain variable comprising an amino acid sequence that is at least 90% identical to any of SEQ ID NOs: 1-6. In some embodiments, provided are nucleic acid molecules encoding a ROR1 antibody or antigen binding fragment thereof comprising a light chain variable region comprising an amino acid sequence that is at least 90% identical to any one of SEQ ID NOs: 7-11. In some embodiments, provided are nucleic acid molecules encoding a ROR1 antibody or antigen binding fragment thereof comprising one or more heavy chain CDRs comprising an amino acid sequence that is at least 90% identical to any one of SEQ ID NOs: 12-23, 67-76, 84-94, 102-110, 119-131. In some embodiments, provided are nucleic acid molecules encoding a ROR1 antibody or antigen binding fragment thereof comprising one or more heavy chain CDRs comprising an amino acid sequence that is at least 90% identical to any one of SEQ ID NOs: 12-23. In some embodiments, provided are nucleic acid molecules encoding a ROR1 antibody or antigen binding fragment thereof comprising one or more light chain CDRs comprising an amino acid sequence that is at least 90% identical to any one of SEQ ID NOs: 24-33, 77-83, 95-101, or 111-118. In some embodiments, provided are nucleic acid molecules encoding a ROR1 antibody or antigen binding fragment thereof comprising one or more light chain CDRs comprising an amino acid sequence that is at least 90% identical to any one of SEQ ID NOs: 24-33.

In some embodiments, provided are nucleic acid molecules encoding antibodies or antigen binding fragments thereof that bind specifically to ROR1, wherein the nucleic acid sequences comprise sequences encoding an amino acid sequence that is at least 90% identical (for example, at least 90%, 91%, 92%, 93%, 94%, 95%, 96%, 97%, 98% or 99% identical) to any of the sequences in Table 1.

In some embodiments, provided are nucleic acid molecules comprising a nucleotide sequence that is at least 90% identical (for example, at least 90%, 91%, 92%, 93%, 94%, 95%, 96%, 97%, 98% or 99% identical) to any of SEQ ID NOs: 34-49.

Also provided herein are nucleic acid molecules encoding a molecule comprising a ROR1 antibody or antigen binding portion thereof (e.g., a CAR) described above. In some embodiments, provided are nucleic acid molecules encoding any of the CARs described herein. In some embodiments, the nucleic acid molecule encodes a CAR that comprises a sequence that is at least 90% identical (for example, at least 90%, 91%, 92%, 93%, 94%, 95%, 96%, 97%, 98% or 99% identical) to SEQ ID NOs: 51, 60, 62, 64, 66. In some embodiments, the nucleic acid molecule comprises a nucleotide sequence that is at least 65% identical (for example, at least 70%, 75%, 80%, 85%, 90%, 91%, 92%, 93%, 94%, 95%, 96%, 97%, 98%, 99%, or 100% identical) to SEQ ID NOs: 50, 59, 61, 63, or 65.

In some embodiments, the nucleic acid molecules encoding the ROR1 antibodies or antigen binding fragments thereof are synthetic sequences designed for expression in a host cell (for example, a human cell).

In some embodiments, the nucleic acid molecules encoding the ROR1 antibodies or antigen binding fragments thereof are operably linked to a promoter capable of directing expression in a bacterial cell or a eukaryotic cell.

The terms “polynucleotide” and “nucleic acid” interchangeably refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. Examples of polynucleotides contemplated herein include single- and double-stranded DNA, single- and double-stranded RNA, and hybrid molecules having mixtures of single- and double-stranded DNA and RNA.

The amino acid sequences of the CDRs and framework regions can be determined using various well-known definitions in the art, e.g., Kabat, Chothia, international ImMunoGeneTics database (IMGT), AbM, and observed antigen contacts (“Contact”). In some embodiments, CDRs are determined according to the IMGT definition. See, Brochet et al., 2008, Nucl. Acids Rex. 36:W503-508. In some embodiments, CDRs are determined by a combination of Kabat, Chothia, and/or Contact CDR definitions.

Also provided herein are DNA constructs comprising a promoter that drives expression in a host cell operably linked to a recombinant nucleic acid molecule comprising a nucleotide sequence that encodes a ROR1 specific antibody or antigen binding fragment thereof.

Also provided herein are vectors, discussed further below, comprising a DNA construct comprising a promoter that drives expression in a host cell operably linked to a recombinant nucleic acid molecule comprising a nucleotide sequence that encodes a ROR1 specific antibody or antigen binding fragment thereof.

Also provided herein are host cells, including bacterial host cells and eukaryotic host cells, comprising a recombinant nucleic acid molecule encoding a ROR1 antibody or antigen binding fragment thereof as described in this disclosure.

Also provided herein are host cells that have been engineered to express and secrete a ROR1 antibody or antigen binding fragment thereof as described in this disclosure. In some embodiments, the cells are suitable for implanting in a patient with cancer. In some embodiments, the cells are animal or human cells, and can be autologous, heterologous, or xenogeneic. In certain embodiments, the cells can be immortalized. In certain embodiments, in order to decrease the chance of an immunological response, the cells can be encapsulated to avoid infiltration of surrounding tissues. In certain embodiments, the encapsulation materials are typically biocompatible, semi-permeable polymeric enclosures or membranes that allow the release of the protein product(s) but prevent the destruction of the cells by a subject's immune system or by other detrimental factors from the surrounding tissues.

Also provided herein are immune cells (e.g., T cells) expressing any of the CARs described herein. In some embodiments, the immune cell expresses the CAR on its surface. In some embodiments, the immune cell comprises a nucleic acid encoding the CAR, wherein the CAR is expressed from the nucleic acid and localized to the immune cell surface. In some embodiments, the immune cell a B-lymphocyte, T-lymphocyte, thymocyte, dendritic cell, natural killer (NK) cell, monocyte, macrophage, granulocyte, eosinophil, basophil, neutrophil, myelomonocytic cell, megakaryocyte, peripheral blood mononuclear cell, myeloid progenitor cell, or a hematopoietic stem cell. In some embodiments, the immune cell is a T cell. In some embodiments, the T cell is a cytotoxic T cell, a helper T cell, a natural killer T cell, a suppressor T cell, a CD8⁺ T cell, a CD4⁺ T cell, a CD8⁺/CD4⁺ T cell, γδ T cell, or a T-regulatory (T-reg) cell.

In some embodiments, immune cells expressing a CAR provided herein are obtained from a subject. Where the immune cells are used to treat (e.g., according to the treatment methods described herein below) the same subject from which they are obtained, they are referred to as autologous cells. Where they are obtained from a different subject, they are referred to as heterologous cells. Immune cells can be isolated from peripheral blood using techniques well known in the art, include Ficoll density gradient centrifugation followed by negative selection to remove undesired cells. In some embodiments, heterologous immune cells useful for the methods provided herein comprise allogeneic T cells, as described in, e.g., Bedoya et al., 2021, Front. Immunol. 12:640082.

In vitro methods are also suitable for preparing monovalent antibodies or antigen binding fragments thereof. Digestion of antibodies to produce fragments thereof, particularly, Fab fragments, can be accomplished using routine techniques known in the art. For instance, digestion can be performed using papain. Examples of papain digestion are described in International Application Publication No. WO 94/29348, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)2 fragment that has two antigen combining sites and is still capable of cross-linking antigen.

The Fab fragments produced in antibody digestion can also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group.

One method of producing proteins comprising the provided antibodies or fragments is to link two or more peptides or polypeptides together by protein chemistry techniques. For example, peptides or polypeptides can be chemically synthesized using currently available laboratory equipment using either Fmoc (9-fluorenylmethyl-oxycarbonyl) or Boc (tert-butyloxycarbonoyl) chemistry (Applied Biosystems, Inc.; Foster City, CA). Those of skill in the art readily appreciate that a peptide or polypeptide corresponding to the antibody provided herein, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group that is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer Verlag Inc., NY). Alternatively, the peptide or polypeptide can by independently synthesized in vivo. Once isolated, these independent peptides or polypeptides may be linked to form an antibody or fragment thereof via similar peptide condensation reactions.

For example, enzymatic ligation of cloned or synthetic peptide segments can allow relatively short peptide fragments to be joined to produce larger peptide fragments, polypeptides or whole protein domains (Abrahmsen et al., Biochemistry, 30:4151 (1991)). Alternatively, native chemical ligation of synthetic peptides can be utilized to synthetically construct large peptides or polypeptides from shorter peptide fragments. This method consists of a two-step chemical reaction (Dawson et al., Science, 266:776 779 (1994)). The first step is the chemoselective reaction of an unprotected synthetic peptide a thioester with another unprotected peptide segment containing an amino terminal Cys residue to give a thioester linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini et al., FEBS Lett. 307:97-101 (1992); Clark et al., J. Biol. Chem. 269:16075 (1994); Clark et al., Biochemistry 30:3128 (1991); Rajarathnam et al., Biochemistry 33:6623-30 (1994)).

Alternatively, unprotected peptide segments can be chemically linked where the bond formed between the peptide segments as a result of the chemical ligation is an unnatural (non-peptide) bond (Schnolzer et al., Science 256:221 (1992)). This technique has been used to synthesize analogs of protein domains as well as large amounts of relatively pure proteins with full biological activity (deLisle et al., Techniques in Protein Chemistry IV. Academic Press, New York, pp. 257-267 (1992)).

Following expression, the antibodies and fragments thereof can be isolated. An antibody or fragment thereof can be isolated or purified in a variety of ways known in the art depending on what other components are present in the sample. Standard purification methods include electrophoretic, molecular, immunological, and chromatographic techniques, including ion exchange, hydrophobic, affinity, and reverse-phase HPLC chromatography. For example, an antibody can be purified using a standard anti-antibody column (e.g., a protein-A or protein-G column). Ultrafiltration and diafiltration techniques, in conjunction with protein concentration, are also useful. See, e.g., Scopes (1994) Protein Purification, 3^{rd} edition, Springer-Verlag, New York City, New York. The degree of purification necessary varies depending on the desired use. In some instances, no purification of the expressed antibody or fragments thereof is necessary.

Methods for determining the yield or purity of a purified antibody or fragment thereof are known in the art and include, e.g., Bradford assay, UV spectroscopy, Biuret protein assay, Lowry protein assay, amido black protein assay, high pressure liquid chromatography (HPLC), mass spectrometry (MS), and gel electrophoretic methods (e.g., using a protein stain such as Coomassie Blue or colloidal silver stain).

### V. Pharmaceutical Compositions and Formulations

The ROR1 antibodies and antigen binding portions thereof described herein, as well as the various molecules comprising said antibodies and antigen binding portions thereof (e.g., CARs) are suitable for administration in vitro or in vivo. Compositions comprising a ROR1 antibody or antigen binding fragment thereof of the present disclosure and a pharmaceutically acceptable carrier (excipient) are provided. In some embodiments, the compositions comprise a CAR comprising the ROR1 antibody or antigen binding fragment thereof. A pharmaceutically acceptable carrier (excipient) is a material that is not biologically or otherwise undesirable, i.e., the material is administered to a subject without causing undesirable biological effects or interacting in a deleterious manner with the other components of the pharmaceutical composition in which it is contained. The carrier is selected to minimize any degradation of the active ingredient and to minimize any adverse side effects in the subject. The compositions may further comprise a diluent, solubilizer, emulsifier, preservative, and/or adjuvant to be used with the methods disclosed herein. Such compositions can be used, for example, in a subject with cancer that would benefit from any of the ROR1 antibodies or antigen binding fragments thereof described herein.

Suitable carriers and their formulations are described in Remington: The Science and Practice of Pharmacy, 21^{st} Edition, Philip P. Gerbino, ed., Lippincott Williams & Wilkins (2006). In certain embodiments, acceptable formulation materials preferably are nontoxic to recipients at the dosages and concentrations employed. In certain embodiments, the formulation material(s) are for subcutaneous and/or intravenous administration. In certain embodiments, the formulation comprises an appropriate amount of a pharmaceutically acceptable salt to render the formulation isotonic. In certain embodiments, the pharmaceutical composition can contain formulation materials for modifying, maintaining or preserving, for example, the pH, osmolality, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. In certain embodiments, the optimal pharmaceutical composition is determined by one skilled in the art depending upon, for example, the intended route of administration, delivery format and desired dosage. See, for example, Remington: The Science and Practice of Pharmacy, 22^{nd} Edition, Lloyd V. Allen, Jr., ed., The Pharmaceutical Press (2014). In certain embodiments, such compositions may influence the physical state, stability, rate of in vivo release and/or rate of in vivo clearance of the ROR1-specific antibody or antigen binding fragment thereof.

In certain embodiments, the primary vehicle or carrier in a pharmaceutical composition can be either aqueous or non-aqueous in nature. For example, in certain embodiments, a suitable vehicle or carrier can be sterile water for injection, physiological saline solution, buffered solutions like Ringer's solution, dextrose solution, or artificial cerebrospinal fluid, possibly supplemented with other materials common in compositions for parenteral administration. In certain embodiments, the saline comprises isotonic phosphate-buffered saline. In certain embodiments, neutral buffered saline or saline mixed with serum albumin are further exemplary vehicles. In certain embodiments, pharmaceutical compositions comprise a pH controlling buffer such phosphate-buffered saline or acetate-buffered saline. In certain embodiments, a composition comprising a ROR1-specific antibody or antigen binding fragment thereof disclosed herein can be prepared for storage by mixing the selected composition having the desired degree of purity with optional formulation agents (see Remington: The Science and Practice of Pharmacy, 22^{nd} Edition, Lloyd V. Allen, Jr., ed., The Pharmaceutical Press (2014)) in the form of a lyophilized cake or an aqueous solution. Further, in certain embodiments, a composition comprising a ROR1-specific antibody or antigen binding fragment thereof disclosed herein can be formulated as a lyophilizate using appropriate excipients. In some instances, appropriate excipients may include a cryo-preservative, a bulking agent, a surfactant, or a combination of any thereof. Exemplary excipients include one or more of a polyol, a disaccharide, or a polysaccharide, such as, for example, mannitol, sorbitol, sucrose, trehalose, and dextran 40. In some instances, the cryo-preservative may be sucrose or trehalose. In some instances, the bulking agent may be glycine or mannitol. In one example, the surfactant may be a polysorbate such as, for example, polysorbate-20 or polysorbate-80.

In certain embodiments, the pharmaceutical composition can be selected for parenteral delivery (e.g., through injection by intravenous, intraperitoneal, intracerebral (intra-parenchymal), intracerebral, intraventricular, intramuscular, subcutaneous, intra-ocular, intraarterial, intraportal, or intralesional routes). Preparations for parenteral administration can be in the form of a pyrogen-free, parenterally acceptable aqueous solution (i.e., water, alcoholic/aqueous solutions, emulsions or suspensions, including saline and buffered media) comprising a ROR1-specific antibody or antigen binding fragment thereof in a pharmaceutically acceptable vehicle. Preparations for parenteral administration can also include non-aqueous solutions, suspensions, and emulsions. Examples of non-aqueous solvents are propylene glycol, polyethylene glycol, vegetable oils such as olive oil, and injectable organic esters such as ethyl oleate. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, anti-oxidants, chelating agents, and inert gases and the like. In certain embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In certain embodiments, hyaluronic acid can also be used, and can have the effect of promoting sustained duration in the circulation. In certain embodiments, implantable drug delivery devices can be used to introduce the desired molecule.

In certain embodiments, the compositions can be selected for inhalation or for delivery through the digestive tract, such as orally. Compositions for oral administration include powders or granules, suspension or solutions in water or non-aqueous media, capsules, sachets, or tables. Thickeners, flavorings, diluents, emulsifiers, dispersing aids or binders are optionally desirable.

In certain embodiments, the compositions can be selected for topical delivery. Formulations for topical administration include ointments, lotions, creams, gels, drops, suppositories, sprays, liquids, and powders. Conventional pharmaceutical carriers, aqueous, powder, or oily bases, thickeners and the like are optionally necessary or desirable.

In certain embodiments, the formulation components are present in concentrations that are acceptable to the site of administration. In certain embodiments, buffers are used to maintain the composition at physiological pH or at a slightly lower pH, typically within a pH range of from about 5 to about 8. For example, the pH may be 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 6.9, 7.0, 7.1, 7.2, 7.3, 7.4, 7.5, 7.6, 7.7, 7.8, 7.9, 8.0, 8.1, 8.2, 8.3, 8.4, or 8.5. In some instances, the pH of the pharmaceutical composition may be in the range of 6.6-8.5 such as, for example, 7.0-8.5, 6.6-7.2, 6.8-7.2, 6.8-7.4, 7.2-7.8, 7.0-7.5, 7.5-8.0, 7.2-8.2, 7.6-8.5, or 7.8-8.3. In some instances, the pH of the pharmaceutical composition may be in the range of 5.5-7.5 such as, for example, 5.5-5.8, 5.5-6.0, 5.7-6.2, 5.8-6.5, 6.0-6.5, 6.2-6.8, 6.5-7.0, 6.8-7.2, or 6.8-7.5. In some instances, the pH of the pharmaceutical composition may be in the range of 4.0-5.5 such as, for example, 4.0-4.3, 4.0-4.5, 4.2-4.8, 4.5-4.8, 4.5-5.0, 4.8-5.2, or 5.0-5.5.

In certain embodiments, a pharmaceutical composition can comprise an effective amount of a ROR1 antibody or antigen binding fragment thereof in a mixture with non-toxic excipients suitable for the manufacture of tablets. In certain embodiments, by dissolving the tablets in sterile water or other appropriate vehicle, solutions can be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions can be selected by one skilled in the art, including formulations involving a ROR1-specific antibody or antigen binding fragment thereof in sustained- or controlled-delivery formulations. In certain embodiments, techniques for formulating a variety of other sustained- or controlled-delivery means, such as liposome carriers, bio-erodible microparticles or porous beads and depot injections, are also known to those skilled in the art. See for example, International Application Publication No. WO/1993/015722, which describes the controlled release of porous polymeric microparticles for the delivery of pharmaceutical compositions. In certain embodiments, sustained-release preparations can include semipermeable polymer matrices in the form of shaped articles, e.g., films, or microcapsules. Sustained release matrices can include polyesters, hydrogels, polylactides (see, e.g., U.S. Pat. Nos. 3,773,919; 5,594,091; 8,383,153; 4,767,628; International Application Publication No. WO1998043615, Calo, E. et al. (2015) Eur. Polymer J 65:252-267 and European Patent No. EP 058,481), including, for example, chemically synthesized polymers, starch based polymers, and polyhydroxyalkanoates (PHAs), copolymers of L-glutamic acid and gamma ethyl-L-glutamate (Sidman et al. (1993) Biopolymers 22:547-556), poly (2-hydroxyethyl-methacrylate) (Langer et al. (1981) J Biomed Mater Res. 15: 167-277; and Langer (1982) Chem Tech 12:98-105), ethylene vinyl acetate (Hsu and Langer (1985) J Biomed Materials Res 19(4):445-460) or poly-D(−)-3-hydroxybutyric acid (European Patent No. EP0133988). In certain embodiments, sustained release compositions can also include liposomes, which can be prepared by any of several methods known in the art. (See, e.g., Eppstein et al. (1985) Proc. Natl. Acad. Sci. USA 82:3688-3692; European Patent No. EP 036,676; and U.S. Pat. Nos. 4,619,794 and 4,615,885).

The pharmaceutical composition to be used for in vivo administration typically is sterile. In certain embodiments, sterilization is accomplished by filtration through sterile filtration membranes. In certain embodiments, where the composition is lyophilized, sterilization using this method can be conducted either prior to or following lyophilization and reconstitution. In certain embodiments, the composition for parenteral administration can be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, once the pharmaceutical composition has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

In still another aspect, unit dose forms comprising a ROR1-specific antibody or antigen binding fragment thereof as described in this disclosure are provided. A unit dose form can be formulated for administration according to any of the routes described in this disclosure. In one example, the unit dose form is formulated for intravenous or intraperitoneal administration. In still another aspect, pharmaceutical packages comprising unit dose forms of a ROR1-specific antibody or antigen binding fragment thereof are provided.

### VII. Kits and Packaging

The ROR1 antibodies and antigen binding fragments thereof disclosed herein are ideally suited for the preparation of a kit. In some embodiments, kits are provided for carrying out any of the methods described herein. The kits of this disclosure may comprise a carrier container being compartmentalized to receive in close confinement one or more containers such as vials, tubes, and the like, each of the containers comprising one of the separate elements to be used in the method.

In some instances, one of the containers may comprise a ROR1 antibody or antigen binding fragment thereof as described in this disclosure that is, or can be, detectably labeled. The kit may also have containers containing buffer(s) and/or a container comprising a reporter-means, such as a biotin-binding protein, such as avidin or streptavidin, bound to a reporter molecule, such as an enzymatic or fluorescent label. For example, a kit for imaging a tumor in a subject with a ROR1-expressing cancer is provided herein. In some embodiments, the kit comprises a container containing a labeled ROR1 antibody or antigen binding fragment thereof. In some embodiments, the kit comprises separate containers containing a ROR1 antibody or antigen binding fragment thereof and a detectable label.

A ROR1 antibody or antigen binding fragment thereof as described in this disclosure for use in treating cancer patients may be delivered in a pharmaceutical package or kit to doctors and cancer patients. Such packaging is intended to improve patient convenience and compliance with the treatment plan. Typically, the packaging comprises paper (cardboard) or plastic. In some embodiments, the kit or pharmaceutical package further comprises instructions for use (e.g., for administering according to a method as described herein).

In some embodiments, a pharmaceutical package or kit comprises unit dose forms of a ROR1 antibody or antigen binding fragment. In some embodiments, the pharmaceutical package or kit further comprises unit dose forms of one or more of a chemotherapeutic agent, a cytotoxic agent, a radiotherapeutic agent, or an immunotherapeutic agent.

In one embodiment, the kit or pharmaceutical package comprises a ROR1 antibody or antigen binding fragment in a defined, therapeutically effective dose in a single unit dosage form or as separate unit doses. The dose and form of the unit dose (e.g., tablet, capsule, immediate release, delayed release, etc.) can be any doses or forms as described herein.

In one embodiment, the kit or pharmaceutical package includes doses suitable for multiple days of administration, such as one week, one month, or three months.

In certain embodiments, kits are provided for producing a single-dose administration unit. In certain embodiments, kits containing single or multi-chambered pre-filled syringes are included. In certain embodiments, kits containing one or more containers of a formulation described in this disclosure are included.

### VIII. Methods of Use

Provided herein are methods to treat, inhibit, or ameliorate cancer in a subject using a ROR1 antibody or antigen binding fragment thereof as described in this disclosure. The methods comprise administering to a subject a pharmaceutically effective amount of a composition comprising an isolated ROR1-specific antibody or antigen binding portion thereof described herein. Also, provided are prognostic and diagnostic methods for cancer based on detection and/or quantitation of ROR1 using a ROR1 antibody or antigen binding fragment as described in this disclosure. Also provided are methods of detecting the presence of ROR1 protein in a sample using the described ROR1 antibodies or antigen binding fragments.

As used throughout, subject can be a vertebrate, more specifically a mammal (e.g., a human, horse, cat, dog, cow, pig, sheep, goat, mouse, rabbit, rat, and guinea pig), birds, reptiles, amphibians, fish, and any other animal. The term does not denote a particular age or sex. Thus, adult and newborn subjects, whether male or female, are intended to be covered. As used herein, patient or subject may be used interchangeably and the term patient or subject includes human and veterinary subjects. The ROR1 antibody or antigen binding portion thereof described herein are useful for treating cancer in humans, including, without limitation, pediatric and geriatric populations, and in animals, e.g., veterinary applications. In one embodiment, the subject is a human.

As used herein the terms “cancer” and “tumor” are used to indicate malignant tissue. The term, “cancer” is also used to refer to the disease associated with the presence of malignant tumor cells in an individual, and the term “tumor” is used to refer to a plurality of cancer cells that are physically associated with each other. Cancer cells are malignant cells that give rise to cancer, and tumor cells are malignant cells that can form a tumor and thereby give rise to cancer.

### A. Methods of Treatment

Provided herein are methods to treat cancer in a subject using a ROR1 antibody or antigen binding fragment thereof as described in this disclosure. In some instances, the ROR1 antibody or antigen binding fragment thereof may directly inhibit growth and induce cell death of cancer cells. In some instances, the ROR1 antibody or antigen binding fragment thereof may inhibit tumor initiation, e.g., by binding to ROR1 expressed by undifferentiated leukemia cells or cancer stem cells. In some instances, the ROR1 antibody or antigen binding fragment thereof may sensitize cancer cells to other cancer therapies (e.g., chemotherapy). In some instances, treating a subject according to the methods described herein inhibits at least one of formation of a tumor, the proliferation of tumor cells, the growth of tumor cells, survival of tumor cells in circulation, or metastasis of tumor cells in the individual. In another embodiment, treating a subject according to the methods described herein may result in tumor growth stasis, reduction of tumor size and, in some instances, elimination of one or more tumors in the subject.

In some instances, the ROR1 antibody or antigen binding fragment thereof itself may not be therapeutic but may be used to target a therapeutic agent to cancer stem cells, as discussed further below. In such instances, the ROR1 antibody or antigen binding fragment thereof need only bind specifically to the ROR1 protein. Thus, in some instances, the ROR1 antibody or antigen binding fragment thereof may be conjugated to a therapeutic pharmaceutical agent, as described below.

Also provided are cancer treatment methods using a CAR comprising a ROR1 antibody or antigen binding fragment thereof as described in this disclosure. In some embodiments, these methods comprise using the CAR to redirect the specificity of an immune effector cell (e.g., a T cell) to target a cancer cell (e.g., a ROR1-expressing cancer cell). Thus, provided herein are methods of stimulating an effector cell-mediated response (such as a T cell-mediated immune response) to a target cell population or tissue comprising cancer cells in a mammal, comprising the step of administering to the mammal an effector cell (such as a T cell) that expresses a CAR as described herein. In some embodiments, “stimulating” an immune cell refers to eliciting an effector cell-mediated response (such as a T cell-mediated immune response), which is different from activating an immune cell. CAR-expressing effector cells described herein can be infused to a subject in need of treatment (e.g., a cancer patient). In some embodiments, the infused cell is able to kill (or lead to the killing of) cancer cells in the subject. Formulations and methods for making CAR-expressing effector cells and using them in therapeutic methods are known in the art (see, e.g., Feins et al., 2019, Am. J. Hematol. 94(S1):S3-S9).

The subject to be treated by any of the methods herein may have one of various of different cancers, including, for example, lymphoma, follicular lymphoma (FL), mantle cell lymphoma (MCL), diffuse large B-cell lymphoma (DLBCL), leukemia, chronic lymphocytic leukemia (CLL), marginal zone lymphoma, breast cancer, ovarian cancer, colon cancer, lung cancer, skin cancer, pancreatic cancer, testicular cancer, bladder cancer, uterus cancer, prostate cancer, or adrenal cancer. In some instances, the subject may have a primary cancer. In other instances, the subject may have metastatic cancer. In some embodiments, the cancer comprises cells that abnormally express ROR1 at a level above basal expression in corresponding normal/non-cancer cells (i.e., a ROR1-expressing cancer). In some embodiments, the subject can have chronic lymphocytic leukemia. In some embodiments, the subject can have mantle cell lymphoma. In some embodiments, the subject can have breast cancer. In some embodiments, the subject can have lung cancer.

In some embodiments, ROR1 expression (e.g., in cancer cells) can be examined by using one or more routine biochemical analyses. In some embodiments, ROR1 expression is determined by detecting protein expression using methods such as mass spectrometry, western blot analysis, flow cytometry, and immunohistochemistry staining. In some embodiments, such methods comprise use of a ROR1 antibody or antigen binding portion thereof (e.g., as described in this disclosure). In some embodiments, ROR1 expression is determined by detecting mRNA levels using methods such as RT-PCR, RNA sequencing, microarray analysis, and northern blot analysis. In some instances, a combination of these methods may be used, or additional methods known in the art may also be used.

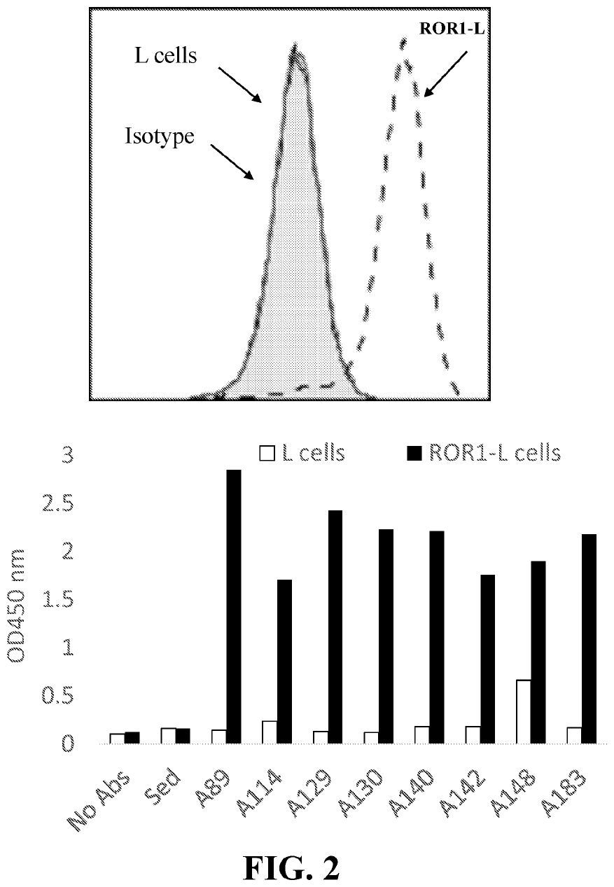

In one embodiment, ROR1 specific antibodies provided herein specifically bind to cells (e.g., L cells, which are mouse fibroblast cells) expressing human ROR1 but not to parental cells that do not express ROR1 (see, e.g., Example 3 and FIG. 2 herein).

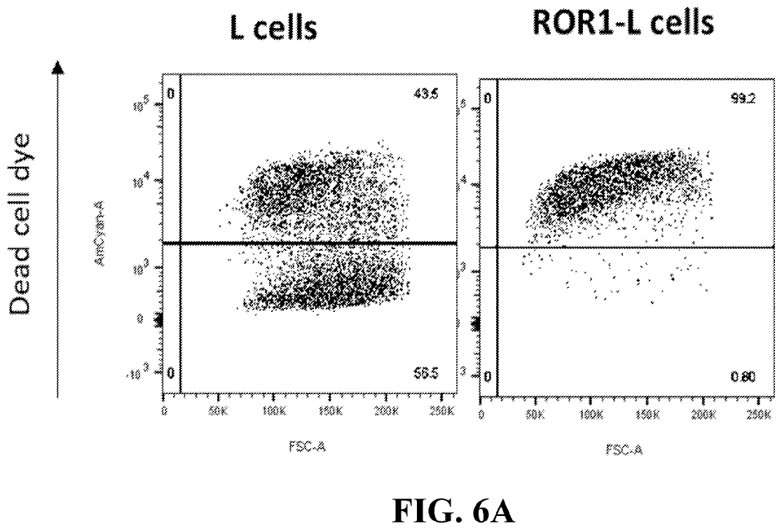

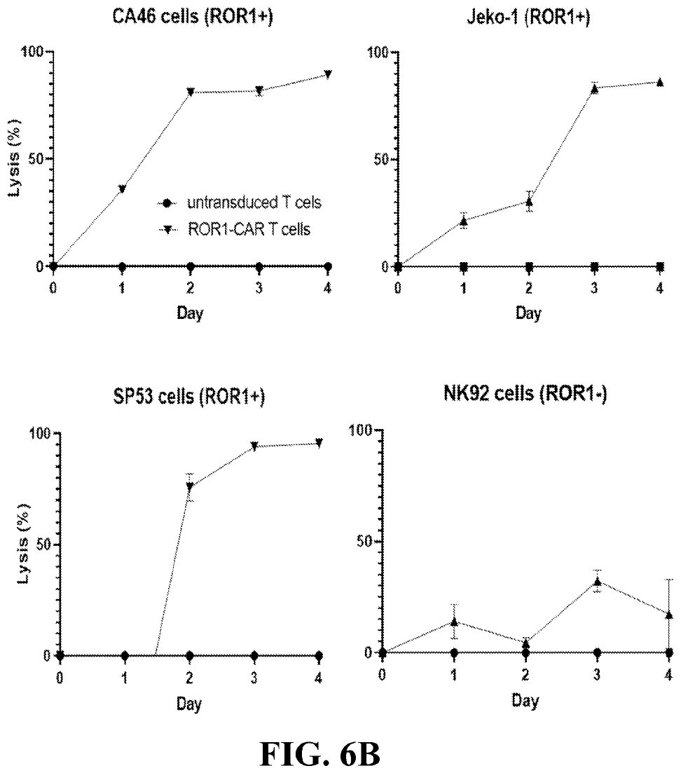

In one embodiment, CAR T cells comprising ROR1 specific antibodies provided herein induce significant lysis of cells expressing human ROR1 (e.g., ROR1-expressing L cells and cells from B-cell lymphoma cell lines including Jeko-1, sp53, and CA46), but not cells that do not express ROR1 (e.g., parental L cells and cells from a ROR1-negative leukemia cell line NK92) (see, e.g., Example 4 and FIGS. 6A-6B herein)

“Treat,” “treatment,” and the like are used herein to generally mean obtaining a desired pharmacologic and/or physiologic effect. “Treating” or “treatment” may refer to any indicia of success in the treatment or amelioration of cancer. “Treating” or “treatment” includes the administration of an agent to impede growth of a cancer, to do one or more of the following: cause a cancer to shrink by weight or volume, extend the expected survival time of the subject, or extend the expected time to progression of the tumor, or the like. The effect of treatment can be compared to an individual or pool of individuals not receiving the treatment, or to the same patient prior to treatment or at a different time during treatment.

The term “administer,” as used herein, refers to a method of delivering agents, compounds, or compositions to the desired site of biological action. The pharmaceutical compositions (e.g., as described above) are prepared for administration in a number of ways, including but not limited to injection, ingestion, transfusion, implantation, or transplantation, depending on whether local or systemic treatment is desired, and on the area to be treated. The preparation of such pharmaceutically acceptable compositions is within the ability of one skilled in the art. The compositions are administered via any of several routes of administration, including topical, oral, parenteral, intravenous, intra-articular, intraperitoneal, intramuscular, subcutaneous, intracavity, intralesional, transdermal, intradermal, intrahepatical, intrathecal, intracranial, rectal, transmucosal, intestinal, ocular, otic, nasal, inhalation, or intrabronchial delivery, or any other method known in the art. In some embodiments, the ROR1 antibody or antigen binding fragment thereof is administered orally, intravenously, or intraperitoneally.

In one aspect, provided is a method of treating a subject with cancer, the method comprising administering to the patient a pharmaceutically effective amount of a composition comprising a ROR1 antibody or antigen binding portion thereof as described in this disclosure. The composition may further comprise a pharmaceutically acceptable carrier.

In some instances, the patient is administered an isolated ROR1 antibody or antigen binding portion thereof. The term “isolated,” as used with reference to a protein (or nucleic acid), denotes that the protein (or nucleic acid) is essentially free of other cellular components with which it is associated in the natural state. It is preferably in a homogeneous state. Purity and homogeneity are typically determined using analytical chemistry techniques such as electrophoresis (e.g., polyacrylamide gel electrophoresis) or chromatography (e.g., high performance liquid chromatography). In some embodiments, an isolated protein (or nucleic acid) is at least 85% pure, at least 90% pure, at least 95% pure, or at least 99% pure.

In some instances, the ROR1 antibody or antigen-binding fragment thereof can be administered via virus-like particles. Virus-like particles (VLPs) comprise viral protein(s) derived from the structural proteins of a virus. Methods for making and using virus like particles are described in, for example, Garcea and Gissmann, Current Opinion in Biotechnology 15:513-7 (2004).

In some instances, the ROR1-specific antibody or antigen-binding fragment thereof can be administered by subviral dense bodies (DBs). DBs transport proteins into target cells by membrane fusion. Methods for making and using DBs are described in, for example, Pepperl-Klindworth et al., Gene Therapy 10:278-84 (2003).

In some instances, the ROR1 antibody or antigen-binding fragment thereof can be administered by tegument aggregates. Methods for making and using tegument aggregates are described in International Publication No. WO 2006/110728.

In another aspect, provided is a method of treating a subject with cancer, the method comprising administering to the patient cells that have been genetically engineered, using methods such as those described herein, to express and secrete a ROR1 antibody or antigen binding portion thereof as described in this disclosure.

In another aspect, provided is a method of treating a subject with cancer, the method comprising administering to the patient immune cells that express a CAR comprising a ROR1 antibody or antigen binding portion thereof as described herein.

In another aspect, provided is a method of treating a subject with cancer, the method comprising administering to the patient a vector comprising a nucleic acid sequence encoding the ROR1 antibody or antigen binding fragment thereof as described in this disclosure.

There are a number of compositions and methods which can be used to deliver the nucleic acid molecules and/or polypeptides to cells, either in vitro or in vivo via, for example, expression vectors. These methods and compositions can largely be broken down into two classes: viral based delivery systems and non-viral based delivery systems. Such methods are well known in the art and readily adaptable for use with the compositions and methods described herein.

As used herein, plasmid or viral vectors are agents that transport the disclosed nucleic acids into the cell without undesired degradation and include a promoter yielding expression of the nucleic acid molecule and/or adapter polypeptide in the cells into which it is delivered. Viral vectors are, for example, Adenovirus, Adeno-associated virus, herpes virus, Vaccinia virus, Polio virus, Sindbis, and other RNA viruses, including these viruses with the HIV backbone. Also preferred are any viral families which share the properties of these viruses which make them suitable for use as vectors. Retroviral vectors, in general are described by Coffin et al., Retroviruses, Cold Spring Harbor Laboratory Press (1997), which is incorporated by reference herein for the vectors and methods of making them. The construction of replication-defective adenoviruses has been described (Berkner et al., J. Virology 61:1213-20 (1987); Massie et al., Mol. Cell. Biol. 6:2872-83 (1986); Haj-Ahmad et al., J. Virology 57:267-74 (1986); Davidson et al., J. Virology 61:1226-39 (1987); Zhang et al., BioTechniques 15:868-72 (1993)). The benefit and the use of these viruses as vectors is that they are limited in the extent to which they can spread to other cell types, since they can replicate within an initial infected cell, but are unable to form new infections viral particles. Recombinant adenoviruses have been shown to achieve high efficiency after direct, in vivo delivery to airway epithelium, hepatocytes, vascular endothelium, CNS parenchyma, and a number of other tissue sites. Other useful systems include, for example, replicating and host-restricted non-replicating vaccinia virus vectors. In some instances, the nucleic acid molecules encoding the ROR1 antibodies or antigen-binding fragments thereof can be delivered via virus-like particles.

Non-viral based delivery methods, can include expression vectors comprising nucleic acid molecules and nucleic acid sequences encoding the adapter polypeptides, wherein the nucleic acids are operably linked to an expression control sequence. Suitable vector backbones include, for example, those routinely used in the art such as plasmids, artificial chromosomes, BACs, YACs, or PACs. Numerous vectors and expression systems are commercially available from such corporations as Novagen (Madison, WI), Clonetech (Pal Alto, CA), Stratagene (La Jolla, CA), and Invitrogen/Life Technologies (Carlsbad, CA). Vectors typically contain one or more regulatory regions. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns.

Preferred promoters controlling transcription from vectors in mammalian host cells may be obtained from various sources, for example, the genomes of viruses such as polyoma, Simian Virus 40 (SV40), adenovirus, retroviruses, hepatitis B virus, and most preferably cytomegalovirus (CMV), or from heterologous mammalian promoters (e.g., β-actin promoter or EF1α promoter), or from hybrid or chimeric promoters (e.g., CMV promoter fused to the β-actin promoter). Of course, promoters from the host cell or related species are also useful herein.

Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 bp in length, and they function in cis. Enhancers usually function to increase transcription from nearby promoters. Enhancers can also contain response elements that mediate the regulation of transcription. While many enhancer sequences are known from mammalian genes (globin, elastase, albumin, fetoprotein, and insulin), typically one will use an enhancer from a eukaryotic cell virus for general expression. Preferred examples are the SV40 enhancer on the late side of the replication origin, the cytomegalovirus early promoter enhancer, the polyoma enhancer on the late side of the replication origin, and adenovirus enhancers.

The promoter and/or the enhancer can be inducible (e.g., chemically or physically regulated). A chemically regulated promoter and/or enhancer can, for example, be regulated by the presence of alcohol, tetracycline, a steroid, or a metal. A physically regulated promoter and/or enhancer can, for example, be regulated by environmental factors, such as temperature and light. Optionally, the promoter and/or enhancer region can act as a constitutive promoter and/or enhancer to maximize the expression of the region of the transcription unit to be transcribed. In certain vectors, the promoter and/or enhancer region can be active in a cell type specific manner. Optionally, in certain vectors, the promoter and/or enhancer region can be active in all eukaryotic cells, independent of cell type. Preferred promoters of this type are the CMV promoter, the SV40 promoter, the beta-actin promoter, the EF1A promoter, and the retroviral long terminal repeat (LTR).

The vectors also can include, for example, origins of replication and/or markers. A marker gene can confer a selectable phenotype, e.g., antibiotic resistance, on a cell. The marker product is used to determine if the vector has been delivered to the cell and once delivered is being expressed. Examples of selectable markers for mammalian cells are dihydrofolate reductase (DHFR), thymidine kinase, neomycin, neomycin analog G418, hygromycin, puromycin, and blasticidin. When such selectable markers are successfully transferred into a mammalian host cell, the transformed mammalian host cell can survive if placed under selective pressure. Examples of other markers include, for example, the E. coli lacZ gene, green fluorescent protein (GFP), and luciferase. In addition, an expression vector can include a tag sequence designed to facilitate manipulation or detection (e.g., purification or localization) of the expressed polypeptide. Tag sequences, such as GFP, glutathione S-transferase (GST), polyhistidine, c-myc, hemagglutinin, or FLAG™ tag (Kodak; New Haven, CT) sequences typically are expressed as a fusion with the encoded polypeptide. Such tags can be inserted anywhere within the polypeptide including at either the carboxyl or amino terminus.

As used herein, an “effective amount” means the amount of an agent that is effective for producing a desired effect in a subject. The actual dose that comprises the effective amount may depend upon the route of administration, the size and health of the subject, the disorder being treated (e.g., cancer), and the like.

In certain embodiments, the effective amount of a pharmaceutical composition comprising a ROR1-specific antibody or antigen binding fragment thereof to be employed therapeutically depends, for example, upon the therapeutic context and objectives. One skilled in the art will appreciate that the appropriate dosage levels for treatment, according to certain embodiments, vary depending, in part, upon the molecule delivered, the indication for which a ROR1-specific antibody or antigen binding fragment thereof is being used, the route of administration, and the size (body weight, body surface or organ size) and/or condition (the age and general health) of the patient. The clinician can titer the dosage and modify the route of administration to obtain the optimal therapeutic effect.

The clinician also selects the frequency of dosing, taking into account the pharmacokinetic parameters of the ROR1-specific antibody or antigen binding fragment thereof in the formulation used. Such pharmacokinetic parameters are well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J Pharm. Sci. 84:1144-1146; Rohatagi (1995) Pharmazie 50:610-613; Brophy (1983) Eur. J Clin. Pharmacol. 24:103-108; the latest Remington's, supra). In certain embodiments, a clinician administers the composition until a dosage is reached that achieves the desired effect. In certain embodiments, the composition can therefore be administered as a single dose or as two or more doses (which may or may not contain the same amount of the desired molecule) over time, or as a continuous infusion via, for example, an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data.

In some cases, the dosage (of the active component) ranges from about 0.0001 to 100 mg/kg, and more usually 0.01 to 20 mg/kg, of the patient's body weight. For example, dosages can be 0.3 mg/kg body weight, 1 mg/kg body weight, 3 mg/kg body weight, 5 mg/kg body weight, 10 mg/kg body weight or within the range of 0.1-20 mg/kg. In certain examples, the ROR1-specific antibodies or antigen-binding fragments thereof can be administered at a dose of 1 mg/kg, 2 mg/kg, 3 mg/kg, 4 mg/kg, or 5 mg/kg once every other day at least four times. An exemplary treatment regime may include administration once per day, once per week, twice a week, once every two weeks, once every three weeks, once every four weeks, once a month, once every 3 months, or once every three to 6 months. In some cases, the treatment comprises administering ROR1-specific antibodies or antigen-binding fragments thereof according to one of the aforementioned dosing regimens for a first period and another of the aforementioned dosing regimens for a second period. In some cases, the treatment discontinues for a period of time before the same or a different dosing regimen resumes. For example, a patient may be on a ROR1-specific antibody dosing regimen for two weeks, off for a week, on for another two weeks, and so on. Dosage regimens for ROR1-specific antibodies or antigen-binding fragments thereof of this disclosure include 0.1 mg/kg body weight, 0.3 mg/kg body weight, 2 mg/kg body weight, 3 mg/kg body weight, or 10 mg/kg via intravenous administration, with the ROR1-specific antibodies or antigen-binding fragments thereof being given using one of the following dosing schedules: (i) every four weeks for six dosages, then every three months; (ii) every three weeks; (iii) 3 mg/kg body weight once followed by 1 mg/kg body weight every three weeks.

In certain embodiments, the route of administration of the pharmaceutical composition is in accord with known methods, e.g., orally, through injection by intravenous, intraperitoneal, intracerebral (intra-parenchymal), intracerebral, intraventricular, intramuscular, subcutaneously, intra-ocular, intraarterial, intraportal, or intralesional routes; by sustained release systems or by implantation devices. In certain embodiments, the compositions can be administered by bolus injection or continuously by infusion, or by implantation device. In certain embodiments, individual elements of a combination therapy may be administered by different routes.

In certain embodiments, the composition can be administered locally, e.g., during surgery or topically. Optionally local administration is via implantation of a membrane, sponge, or another appropriate material onto which the desired molecule has been absorbed or encapsulated. In certain embodiments, where an implantation device is used, the device can be implanted into any suitable tissue or organ, and delivery of the desired molecule can be via diffusion, timed-release bolus, or continuous administration.

In certain embodiments, it can be desirable to use a pharmaceutical composition comprising a ROR1 antibody or antigen binding fragment thereof in an ex vivo manner. In such instances, cells that have been removed from a subject may be exposed to a pharmaceutical composition comprising a ROR1 antibody or antigen binding fragment thereof after which the cells are subsequently implanted back into the subject.

In some instances, the provided methods may include administering to the subject a ROR1-specific antibody or antigen binding fragment thereof that is conjugated to a therapeutic agent. The therapeutic agent may be at least one of a cytotoxic agent, a chemotherapeutic agent, or an immunosuppressive agent. Such therapeutic agents are described below.

In some instances, the provided methods may include administering a ROR1-specific antibody or antigen binding fragment thereof and a second form of cancer therapy to the subject. The second form of cancer therapy may include a cytotoxic agent, a chemotherapeutic agent, an immunosuppressive agent (including immune checkpoint inhibitors), or radiation therapy. In some embodiments, the second form of cancer therapy is an antibody (e.g., a monoclonal antibody). Monoclonal antibodies which may be administered as a second form of cancer therapy include, but are not limited to, rituximab (e.g., for treatment of B-cell lymphomas), trastuzumab (e.g., for treatment of breast cancer), and cetuximab (e.g., for treatment of lung cancer).

In some instances, the ROR1 antibody or antigen binding fragment thereof can be labeled, conjugated, or fused with a therapeutic agent or diagnostic agent (such as an imaging agent). The linkage can be covalent or noncovalent (e.g., ionic). Such antibodies and antibody fragments are referred to antibody-drug conjugates (ADC) or immunoconjugates. The antibody conjugates are useful for the local delivery of therapeutic agents, particularly cytotoxic or cytostatic agents, i.e. drugs to kill or inhibit tumor cells in the treatment of cancer allows targeted delivery of the drug moiety to tumors, and intracellular accumulation therein, where systemic administration of these unconjugated drug agents may result in unacceptable levels of toxicity to normal cells as well as the tumor cells sought to be eliminated. Therapeutic agents include but are not limited to toxins, including but not limited to plant and bacterial toxins, small molecules, peptides, polypeptides and proteins. Genetically engineered fusion proteins, in which genes encoding for an antibody, or fragments thereof including the Fv region, or peptides can be fused to the genes encoding a toxin to deliver a toxin to the target cell are also provided. As used herein, a target cell or target cells are ROR1 positive cells.

In some embodiments, the ROR1 antibody or antigen binding fragment thereof is conjugated to a moiety that specifically binds to an immune cell. In some embodiments, provided is a bispecific antibody comprising a ROR1 antibody or antigen binding fragment thereof as described herein and an antibody or antigen binding fragment thereof that specifically binds to an immune cell. In some embodiments, the bispecific antibody comprises a ROR1-specific antibody or antigen-binding portion thereof and an antibody moiety that specifically binds to T cells. Such a molecule is referred to as a bispecific T cell engager and may induce T cell-mediated cytotoxicity of ROR1-expressing cancer cells (see, e.g., Zhou et al., 2021, Biomarker Research 9:38). In some embodiments, the bispecific antibody comprises a ROR1-specific antibody or antigen-binding portion thereof and an antibody moiety that specifically binds to natural killer cells (NK cells). Such a molecule is referred to as a NK cell engager and may induce NK cell-mediated cytotoxicity of ROR1-expressing cancer cells (see, e.g., Demaria et al., 2021, European Journal of Immunology 51(8):1934-1942).

Other examples of therapeutic agents include chemotherapeutic agents, a radiotherapeutic agent, and immunotherapeutic agent, as well as combinations thereof. In this way, the antibody or peptide complex delivered to the subject can be multifunctional, in that it exerts one therapeutic effect by binding to the ROR1 protein and a second therapeutic effect by delivering a supplemental therapeutic agent.

The therapeutic agent can act extracellularly, for example by initiating or affecting an immune response, or it can act intracellularly, either directly by translocating through the cell membrane or indirectly by, for example, affecting transmembrane cell signaling. The therapeutic agent is optionally cleavable from the ROR1 antibody or antigen binding fragment thereof. Cleavage can be autolytic, accomplished by proteolysis, or affected by contacting the cell with a cleavage agent.

In some instances, the therapeutic agent is a cytotoxic agent. A cytotoxin or cytotoxic agent includes any agent that is detrimental to cells. Examples of toxins or toxin moieties include diphtheria, ricin, streptavidin, and modifications thereof. Additional examples include paclitaxel, cisplatin, carboplatin, cytochalasin B, gramicidin D, ethidium bromide, emetine, etoposide, tenoposide, colchicin, dihydroxy anthracin dione, mitoxantrone, mithramycin, actinomycin D, 1-dehydrotestosterone, glucocorticoids, procaine, tetracaine, lidocaine, propranolol, puromycin and analogs or homologs thereof. Therapeutic agents include, but are not limited to, antimetabolites (e.g., methotrexate, 6-mercaptopurine, 6-thioguanine, cytarabine, 5-fluorouracil, decarbazine), alkylating agents (e.g., mechlorethamine, thiotepa, chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e. g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). Cytotoxic peptides such as auristatin (antineoplastic) peptides auristatin E (AE) and monomethylauristatin (MMAE), which are synthetic analogs of dolastatin, may also be conjugated to the ROR1-specific antibody or antigen binding fragment thereof. In some instances, the ROR1-specific antibody or antigen binding fragment thereof may be conjugated to a radioactive metal ion.

As referred to herein, a chemotherapeutic agent is a chemical compound useful in the treatment of cancer. Examples of chemotherapeutic agents include erlotinib (such as TARCEVA®, Genentech/OSI Pharm.), bortezomib (such as VELCADE®, Millenium Pharm.), fulvestrant (such as FASLODEX®, AstraZeneca), sutent (such as SU11248, Pfizer), letrozole (such as FEMARA®, Novartis), imatinib mesylate (such as GLEEVEC®, Novartis), PTK787/ZK222584 (Novartis), oxaliplatin (such as ELOXATIN®, Sanofi), 5-fluorouracil (5-FU), leucovorin, rapamycin (also known as sirolimus) (such as RAPAMUNE®, Wyeth), lapatinib (such as TYKERB®, GSK572016, GlaxoSmithKline), lonafarnib (such as SCH 66336), sorafenib (such as BAY43-9006, Bayer Labs.), capecitabine (such as XELODA®, Roche), docetaxel (such as TAXOTERE®), and gefitinib (such as IRESSA®, Astrazeneca), AG1478, AG1571 (such as SU 5271; Sugen Inc.), alkylating agents such as thiotepa and cyclosphosphamide (such as CYTOXAN®); alkyl sulfonates such as busulfan, improsulfan and piposulfan; aziridines such as benzodopa, carboquone, meturedopa, and uredopa; ethylenimines and methylamines including altretamine, triethylenemelamine, triethylenephosphoramide, triethylenethiophosphoramide and trimethylomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, particularly calicheamicin γ₁^{I} and calicheamicin θ₁^{I}); dynemicin, including dynemicin A; bisphosphonates, such as clodronate; an esperamicin; as well as neocarzinostatin chromophore and related chromoprotein enediyne antibiotic chromophores), aclacinomysins, actinomycin, anthramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (such as ADRIAMYCIN®, including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; Trametes versicolor polysaccharide-K (Krestin, PSK) (JHS Natural Products, Eugene, OR); razoxane; rhizoxin; sizofiran; spirogermanium; tenuazonic acid; triaziquone; 2,2′, 2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; cytarabine (cytosine arabinoside, “Ara-C”); cyclophosphamide; thiotepa; taxoids, e.g., paclitaxel (such as TAXOL®, Bristol-Myers Squibb Oncology, Princeton, N.J.), ABRAXANE™ (a Cremophor-free, albumin-engineered nanoparticle formulation of paclitaxel (American Pharmaceutical Partners, Schaumberg, IL)), and doxetaxel (such as TAXOTERE®, Rhône-Poulenc Rorer, Antony, France); chloranbucil; gemcitabine (such as GEMZAR®); 6-thioguanine; mercaptopurine; methotrexate; platinum analogs such as cisplatin and carboplatin; vinblastine; platinum; etoposide (VP-16); ifosfamide; mitoxantrone; vincristine; vinorelbine (such as NAVELBINE®); novantrone; teniposide; edatrexate; daunomycin; aminopterin; xeloda; ibandronate; CPT-11; topoisomerase inhibitor RFS 2000; difluorometlhylomithine (DMFO); retinoids such as retinoic acid; capecitabine; and pharmaceutically acceptable salts, acids or derivatives of any of the above.

Chemotherapeutic agents, as used herein, also refers to (i) anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY 117018, onapristone, and toremifene (such as FARESTON®); (ii) aromatase inhibitors that inhibit the enzyme aromatase, which regulates estrogen production in the adrenal glands, such as, for example, 4(5)-imidazoles, aminoglutethimide, megestrol acetate (such as MEGASE®), exemestane (such as AROMASIN®), formestanie, fadrozole, vorozole (such as RIVISOR®), letrozole (such as FEMARA®), and anastrozole (such as ARIMIDEX®); (iii) anti-androgens such as flutamide, nilutamide, bicalutamide, leuprolide, and goserelin; as well as troxacitabine (a 1,3-dioxolane nucleoside cytosine analog); (iv) aromatase inhibitors; (v) protein kinase inhibitors; (vi) lipid kinase inhibitors; (vii) antisense oligonucleotides, particularly those which inhibit expression of genes in signaling pathways implicated in aberrant cell proliferation, such as, for example, PKC-alpha, Ralf and H-Ras; (viii) VEGF receptor and angiogenesis inhibitors (including ribozymes such as ANGIOZYME®) and a HER2 expression inhibitor; (ix) vaccines such as gene therapy vaccines, for example, ALLOVECTIN-7® vaccine (plasmid/lipid complex containing the DNA sequences encoding HLA-B7 and ß2 microglobulin), LEUVECTIN® vaccine (plasmid DNA expression vector encoding interleukin-2 (IL-2) complexed with a lipid delivery vehicle (DMRIE/DOPE)), and VAXID® vaccine (patient-specific naked DNA vaccine); IL-2 or aldesleukin (such as PROLEUKIN®); topoisomerase 1 inhibitors (such as TOPOTECAN®); gonadotropin-releasing hormone antagonists (such as ABARELIX®); (x) anti-angiogenic agents such as bevacizumab (such as AVASTIN®, Genentech); and (xi) pharmaceutically acceptable salts, acids or derivatives of any of the above.

In some instances, the treatment methods provided herein may further comprise administering an immunosuppressive agent such as an immune checkpoint inhibitor as part of the method. These treatments work by “taking the brakes off” the immune system (are immunosuppressive), allowing it to mount a stronger and more effective attack against cancer. Several different types of checkpoint inhibitors, targeting different checkpoints or “brakes” on immune cells, are currently in use. Exemplary immunosuppressive agents are PD-1 inhibitors (such as nivolumab and pembrolizumab), PD-L1 inhibitors (such as atezolizumab, durvalumab, and avelumab), and CTLA-4 inhibitors (such as ipilimumab). In one example, the second form of cancer therapy comprises a PD-L1 inhibitor, a PD-1 inhibitor, or a CTLA4 inhibitor. In some instances, combinations of such inhibitors can be administered. In some instances, the PD-L1 inhibitor, the PD-1 inhibitor, and/or the CTLA4 inhibitor may be an inhibitory antibody that binds specifically to PD-L1, PD-1, or CTLA4, respectively.

In some instances, the treatment methods provided herein may further comprise administering radiation therapy to the subject. Radiation therapy uses high-energy radiation to shrink tumors and kill cancer cells. X-rays, gamma rays, and charged particles are types of radiation used for cancer treatment. The radiation may be delivered by a machine outside the body (external-beam radiation therapy), or it may come from radioactive material placed in the body near cancer cells (internal radiation therapy, also called brachytherapy). Systemic radiation therapy uses radioactive substances, such as radioactive iodine, that travel in the blood to kill cancer cells.

### B. Diagnostic Methods

In another aspect, provided are methods of assessing eligibility of a subject for inclusion in or exclusion from a clinical trial of a ROR1 targeted therapy using a ROR1 antibody or antigen binding fragment thereof. The method comprises (a) measuring in a tumor sample from a subject the amount of ROR1; (b) determining if a subject has a cancer characterized as having a high level of ROR1 expression; and (c) indicating that the subject is eligible for a clinical trial of a ROR1 targeted therapy if the subject's cancer is characterized as having a high level of ROR1 expression, i.e., above a predetermined threshold or that the subject is ineligible for a clinical trial of ROR1 targeted therapy if the subject's cancer is characterized as having a low level of ROR1 expression, i.e., below a predetermined threshold. In some instances, the threshold level is a median amount of ROR1 determined in a reference population of patients having the same kind of cancer as the subject. In another instance, the threshold level is an optimal amount of ROR1 determined in a reference population of patients having the same kind of cancer as the subject. “Optimal cutoff” as used herein, refers to the value of a predetermined measure on subjects exhibiting certain attributes that allow the best discrimination between two categories of an attribute. For example, finding a value for an optimal cutoff that allows one to best discriminate between two categories (subgroups) of patients for determining at least one of overall survival, time to disease progression, progression-free survival, and likelihood to respond to treatment (e.g., based on clinical assessment using the RECIST criteria, e.g., Eisenhauer, E. A., et al., Eur. J. Cancer 45:228-247 (2009) or the like as recognized in the medical field). Optimal cutoffs are used to separate the subjects with values lower than or higher than the optimal cutoff to optimize the prediction model, for example, without limitation, to maximize the specificity of the model, maximize the sensitivity of the model, maximize the difference in outcome, or minimize the p-value from hazard ratio or a difference in response.

In another aspect, provided are methods for assessing responsiveness of a subject with cancer to a ROR1 antibody or antigen binding fragment thereof comprising: (a) measuring in a tumor sample from a subject the amount of ROR1; (c) determining if the subject has a cancer characterized as having a high level of ROR1 expression; and (d) indicating that the subject is more likely to respond to the ROR1 antibody or antigen binding fragment thereof if the subject's cancer is characterized as having a high level of ROR1 expression. Conversely, if the subject's cancer is characterized as having a low level of ROR1 expression, the subject is less likely to respond to a ROR1 antibody or antigen binding fragment thereof. In some instances, the amount of ROR1 in the tumor sample is measured using a ROR1 antibody or antigen binding fragment thereof as described herein.

In another aspect, provided are methods to diagnose cancer in a subject. Specifically, the diagnosis may be of a ROR1-expressing cancer. The method may comprise measuring in a sample from a subject the amount of ROR1 and diagnosing the subject with cancer if the amount of ROR1 expression in the sample is high. In some instances, the method may comprise (a) measuring in a tumor sample from a subject the amount of ROR1 using a ROR1 antibody or antigen binding fragment thereof, and (c) determining if the subject has a cancer characterized as having a high level of ROR1 expression. Conversely, if the amount of ROR1 expression in the sample or the subject's cancer low level, the subject may not be diagnosed with cancer or may not be diagnosed with a ROR1-expressing cancer.

In some instances, to diagnose cancer in a subject, or to characterize a subject's cancer, a biopsy is typically taken from a subject having an abnormal tissue growth, such as a tumor. Samples may be formalin-fixed, paraffin-embedded tissue samples obtained from the subject's cancer (tumor). In other instances, such as where circulating tumor cells are to be assessed, the sample from the subject is a blood, plasma, or lymph sample. Typically, the tissue or cells of the patient sample are examined under a microscope in order to confirm the diagnosis and/or assess information about the tumor. In some cases, additional tests may need to be performed on the proteins, DNA, and/or mRNA of the cells in the ample to verify the diagnosis or characterization.

### C. Methods of Detecting ROR1

In another aspect, provided are methods for detecting the presence of ROR1-expressing cells in a biological sample comprising: (a) contacting said sample with a composition comprising an isolated ROR1 antibody or antigen binding portion thereof as described in this disclosure; and (b) detecting an amount of binding of the isolate antibody or antigen binding portion thereof as a determination of the presence of said ROR1-expressing cells. In some embodiments, the biological sample comprises a tumor sample. In some embodiments, the composition further comprises a pharmaceutically acceptable carrier.

In some embodiments, ROR1 expression in cancer cells can be examined by using one or more routine biochemical analyses. In some embodiments, ROR1 expression is determined by detecting protein expression using methods such as Western blot analysis, flow cytometry, and immunohistochemistry staining using a ROR1 antibody or antigen binding portion thereof as described in this disclosure. In some instances, a combination of these methods may be used or additional methods may also be used such as microarray analysis and RT-PCR.

In some instances, a threshold amount of ROR1 protein expression is used to characterize ROR1 expression as either high or low. A high level of ROR1 protein expression refers to a measure of ROR1 protein expression above a particular threshold. For example, the threshold may be a normal, an average, or a median amount of ROR1 protein expression as measured in a particular set of samples, referred to as a reference population. In some instances, the reference population may be a population of normal/healthy subjects. In other instances, the reference population may be a population of subjects having a particular type of cancer (the same type of cancer that the subject being assessed has). A low level of ROR1 expression refers to the converse of the above. For example, the threshold may be determined by identifying two distinct subgroups in the reference population by dividing samples around a mathematically determined point, such as, without limitation, a median, thus creating a subgroup whose measure is high (i.e., higher than the median) and another subgroup whose measure is low.

Also provided are methods of imaging a tumor in a subject with a ROR1-expressing cancer, the method comprising administering to the subject an isolated antibody or antigen binding portion thereof that is specific for ROR1 that is conjugated to an imaging label, and detecting the imaging label in the subject. Imaging methods may be used to assess tumor size and changes in tumor size over or after the course of a treatment administered to the subject. The methods may be useful to assess response of the subject to an administered treatment. In some instances, the methods may be useful to grade the subject's cancer.

Also provided are methods of monitoring response of a subject with a ROR1-expressing cancer to cancer therapy. The methods include administering to the subject a ROR1-specific antibody or antigen-binding fragment thereof conjugated to an imaging label at a first time point prior to the subject before the subject receives cancer therapy, detecting the imaging label in the subject to obtain a first image of the tumor, administering to the subject a ROR1-specific antibody or antigen-binding fragment thereof conjugated to an imaging label at a second time point after the subject receives cancer therapy, detecting the imaging label in the subject to obtain a second image of the tumor; and comparing the first image to the second image to determine whether a change in tumor size has occurred. In some instances, the steps of administering to the subject a ROR1-specific antibody or antigen-binding fragment thereof conjugated to an imaging label at a first time point after the subject receives cancer therapy, detecting the imaging label in the subject to obtain a second image of the tumor; and comparing the first image to the second image to determine whether a change in tumor size has occurred may be repeated at a third time point (or additional time points) after the subject receives cancer therapy.

In some embodiments, the isolated antibody or antigen binding portion thereof administered at the two or more time points is identical. In some embodiments, the isolated antibody or antigen binding portion thereof administered at the first time point differs from the isolated antibody or antigen binding portion thereof administered at the second time point. In some embodiments, the imaging label conjugated to the isolated antibody or antigen binding portion thereof administered at the two or more time points is identical. In some embodiments, the imaging label conjugated to the isolated antibody or antigen binding portion thereof administered at the first time point differs from the isolated antibody or antigen binding portion thereof administered at the second time point.

In one embodiment, a subject is administered a labeled ROR1 antibody or antigen binding fragment thereof as described in this disclosure that is conjugated to an imaging agent. The labeled ROR1 antibody or antigen binding fragment thereof is allowed to incubate in vivo and bind to ROR1 in the subject's tissues. The imaging label is thereby localized to tumor cells or tissues, and the localized imaging label is detected using an appropriate imaging device as known to those skilled in the art.

For imaging purposes, the ROR1 antibody or antigen-binding fragment thereof may be conjugated to an imaging agent. For example, the ROR1 antibody or antigen-binding fragment thereof may be labelled for use in radionuclide imaging. In particular, the agent may be directly or indirectly labelled with a radioisotope. Examples of radioisotopes that may be used are: ²⁷⁷Ac, ²¹¹At, ¹²⁸Ba, ¹³¹Ba, ⁷Be, ²⁰⁴Bi, ²⁰⁵Bi, ²⁰⁶Bi, ⁷⁶Br, ⁷⁷Br, ⁸²Br, ¹⁰⁹Cd, ⁴⁷Ca, ¹¹C, ¹⁴C, ³⁶Cl, ⁴⁸Cr, ⁵¹Cr, ⁶²Cu, ⁶⁴Cu, ⁶⁷Cu, ¹⁶⁵Dy, ¹⁵⁵Eu, ¹⁸F, ¹⁵³Gd, ⁶⁶Ga ⁶⁷Ga, ⁶⁸Ga, ⁷²Ga, ¹⁹⁸Au, ³H, ¹⁶⁶Ho, ¹¹¹In, ^{113m}In, ^{115m}In, ¹²³I, ¹²⁵I, ¹³¹I, ¹⁸⁹Ir, ^{191m}Ir, ¹⁹²Ir, ¹⁹⁴Ir, ⁵²Fe, ⁵⁵Fe, ⁵⁹Fe, ¹⁷⁷Lu, ¹⁵O, ^{191m-191}Os, ¹⁰⁹Pd, ³²P, ³³P, ⁴²K, ²²⁶Ra, ¹⁸⁶Re, ¹⁸⁸Re, ^{82m}Rb, ¹⁵³Sm, ⁴⁶Sc, ⁴⁷Sc, ⁷²Se, ⁷⁵Se, ¹⁰⁵Ag, ²²Na, ²⁴Na, ⁸⁹Sr, ³⁵S, ³⁸S, ¹⁷⁷Ta, ⁹⁶Tc, ^{99m}Tc, ²⁰¹Tl, ²⁰²Tl, ¹¹³Sn, ^{117m}Sn, ¹²¹Sn, ¹⁶⁶Yb, ¹⁶⁹Yb, ¹⁷⁵Yb, ⁸⁸Y, ⁹⁰Y, ⁶²Zn and ⁶⁵Zn. Preferably the radioisotope is ¹³¹I, ¹²⁵I, ¹²³I, ¹¹¹I, ^{99m}Tc, ⁹⁰Y, ¹⁸⁶Re, ¹⁸⁸Re, ³²P, ¹⁵³Sm, ⁶⁷Ga, ²⁰¹Tl, ⁷⁷Br, or ¹⁸F, and is imaged with a photoscanning device. Procedures for labeling biological agents with the radioactive isotopes are generally known in the art.

The imaging agent may carry a bioluminescent or chemiluminescent label. Such labels include polypeptides known to be fluorescent, bioluminescent or chemiluminescent, or, that act as enzymes on a specific substrate (reagent), or can generate a fluorescent, bioluminescent or chemiluminescent molecule. Examples of bioluminescent or chemiluminescent labels include luciferases, aequorin, obelin, mnemiopsin, berovin, a phenanthridinium ester, and variations thereof and combinations thereof. A substrate for the bioluminescent or chemiluminescent polypeptide may also be used in imaging. For example, the chemiluminescent polypeptide can be luciferase and the reagent luciferin. A substrate for a bioluminescent or chemiluminescent label can be administered before, at the same time (e.g., in the same formulation), or after administration of the agent.

The imaging agent may include a paramagnetic compound, such as a polypeptide chelated to a metal (e.g., a metalloporphyrin). The paramagnetic compound may also include a monocrystalline nanoparticle, e.g., a nanoparticle including a lanthanide (e.g., Gd) or iron oxide; or, a metal ion such as a lanthanide. Examples of elements that are useful in magnetic resonance imaging include gadolinium, terbium, tin, iron, or isotopes thereof.

Whole body imaging techniques using radioisotope labeled agents can be used for locating diseased cells and tissues (e.g., primary tumors and tumors which have metastasized). In some cases, the labeled agents for locating the tumor tissue or cells are administered intravenously. The bio-distribution of the label can be monitored by scintigraphy, and accumulations of the label are related to the presence of ROR1 or other tumor markers. Whole body imaging techniques are described in, e.g., U.S. Pat. Nos. 4,036,945 and 4,311,688.

An image according to this disclosure can be generated by computer assisted tomography (CAT), magnetic resonance spectroscopy (MRS) image, magnetic resonance imaging (MRI), positron emission tomography (PET), single-photon emission computed tomography (SPECT), or bioluminescence imaging (BLI) or equivalent.

Computer assisted tomography (CAT) and computerized axial tomography (CAT) systems and devices well known in the art can be used to generate an image. (See, for example, U.S. Pat. Nos. 6,151,377; 5,946,371; 5,446,799; 5,406,479; 5,208,581; and 5,109,97.) The imaging methods may also utilize animal imaging modalities, such as MICROCAT™ (ImTek, Inc.).

Magnetic resonance imaging (MRI) systems and devices well known in the art can be used for imaging. For a description of MRI methods and devices, see, for example, U.S. Pat. No. 6,151,377. MRI and supporting devices are commercially available, for example, from Bruker Medical GMBH; Caprius; Esaote Biomedica; Fonar; GE Medical Systems (GEMS); Hitachi Medical Systems America; Intermagnetics General Corporation; Lunar Corp.; MagneVu; Marconi Medicals; Philips Medical Systems; Shimadzu; Siemens; Toshiba America Medical Systems; including imaging systems, by, e.g., Silicon Graphics.

Positron emission tomography imaging (PET) systems and devices well known in the art can be used for imaging. For example, an imaging method of this disclosure may use the system designated Pet VI located at Brookhaven National Laboratory. For descriptions of PET systems and devices, see, for example, U.S. Pat. No. 6,151,377. Animal imaging modalities such as micro-PETs (Concorde Microsystems, Inc.) can also be used.

Single-photon emission computed tomography (SPECT) systems and devices well known in the art can be used for imaging. (See, for example, U.S. Pat. Nos. 6,115,446; 6,072,177; 5,608,221; 5,600,145; 5,210,421; 5,103,098.) Imaging methods may also use animal imaging modalities, such as micro-SPECTs.

Sensitive photon detection systems can be used to detect bioluminescent and fluorescent proteins externally; see for example, Contag (2000), Neoplasia 2:41-52; adn Zhang (1994), Clin. Exp. Metastasis, 12:87-92. The imaging methods of the disclosure can be practiced using any such photon detection device, for example, an intensified charge-coupled device (ICCD) camera coupled to an image processor. Photo detection devices are also commercially available from Xenogen, Hamamatsue.

Disclosed herein are materials, compositions, and methods that can be used for, can be used in conjunction with or can be used in preparation for the disclosed embodiments. These and other materials are disclosed herein, and it is understood that when combinations, subsets, interactions, groups, etc. of these materials are disclosed that while specific reference of each various individual and collective combinations and permutations of these compositions may not be explicitly disclosed, each is specifically contemplated and described herein. For example, if a method is disclosed and discussed, and a number of modifications that can be made to a number of molecules included in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are various additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties. The following description provides further non-limiting examples of thwe disclosed compositions and methods.

### EXAMPLES

The following examples are offered to illustrate, but not to limit, the claimed invention.

### Example 1. Materials and Methods

Assessment of ROR1 transcripts in normal tissues. Human Total RNA Survey Panel containing RNA from 22 normal human tissues was obtained from Applied Biosystems, Foster City, CA. About 10 μg of total RNA from each source was reverse transcribed into cDNA with Superscript III kit (Invitrogen). Quantitative PCR was performed with ROR1 and 3-actin primers using the following conditions: 50° C. for 2 min, 94° C. for 10 min, followed by 94° C. for 15 sec, 60° C. for 60 sec for 40 cycles on Applied Biosystems StepOne™/StepOnePlus™ Real-Time PCR System. The expression of ROR1 mRNA relative to the β-actin mRNA was calculated in each sample.

Generation of anti-human ROR1 monoclonal antibodies. Human ROR1 cDNA was stably transfected into L cells, a mouse fibroblast cell line, and the ROR1-expressing L cells were used to immunize BALB/c mice. Splenocytes from immunized mice were isolated and hybridized with mouse myeloma cells to generate mouse monoclonal antibodies targeting human ROR1 by hybridoma technology.

Staining of B-cell lymphoma cell lines. Anti-ROR1 antibodies were fluorochrome conjugated with AF647 and used to stain B-cell lymphoma cell lines as well as normal donor peripheral blood mononuclear cells (PBMC) along with antibodies against CD3, CD20, CD14, and CD56. Stained cells were analyzed by flow cytomtery.

Generation of an ROR1-specific CAR construct. The VH and VL sequences of the ROR1 antibody, clone A129, were converted to single chain variable fragment (scFv) and a CAR was generated using CD8α (CD8 alpha) hinge and transmembrane domain, and 4-1BB and CD3ζ (CD3 zeta) signaling domains. The entire CAR sequence was incorporated into a lentivirus vector (pLVMG).

Transduction of normal donor T cells with ROR1-specific CAR. Normal donor T cells were purified from buffy coat, activated for 2-3 days with anti-CD3, CD28, CD2 antibodies in the presence of IL-2, and then transduced with ROR1 CAR containing lentivirus. The transduction efficiency was assessed by staining with ROR1 extra cellular domain protein-conjugated FITC (ACROBiosystems, USA) and analysis by flow cytometry.

Cytotoxic assay of ROR1-specific CAR-T cells against B-cell lymphoma cell lines. Jeko-1 and SP53 (mantle cell lymphoma cell lines), and CA46 (Burkitt Lymphoma cell line) were labeled with CellTrace™ Far Red cell staining kit (Thermofisher), and NK-cell lymphoma cell line NK92 (NK-cell leukemia cell line) was stained with CFSE (Thermofisher). The B-cell lymphoma and NK92 cell lines were then co-cultured with ROR1-specific CAR-transduced T cells at 1:1:1 Effector:Tumor 1: Tumor 2 (E:T1:T2) ratio. The percentage of dead cells was determined by flow cytometry on days 1, 2, 3 and 4 after staining for dead cells with Aqua (Thermofisher). Untransduced T cells or tumor alone were used as negative controls.

### Example 2. ROR1 Expression is Restricted in Normal Tissues and is Overexpressed in CLL and Other B-Cell Malignancies

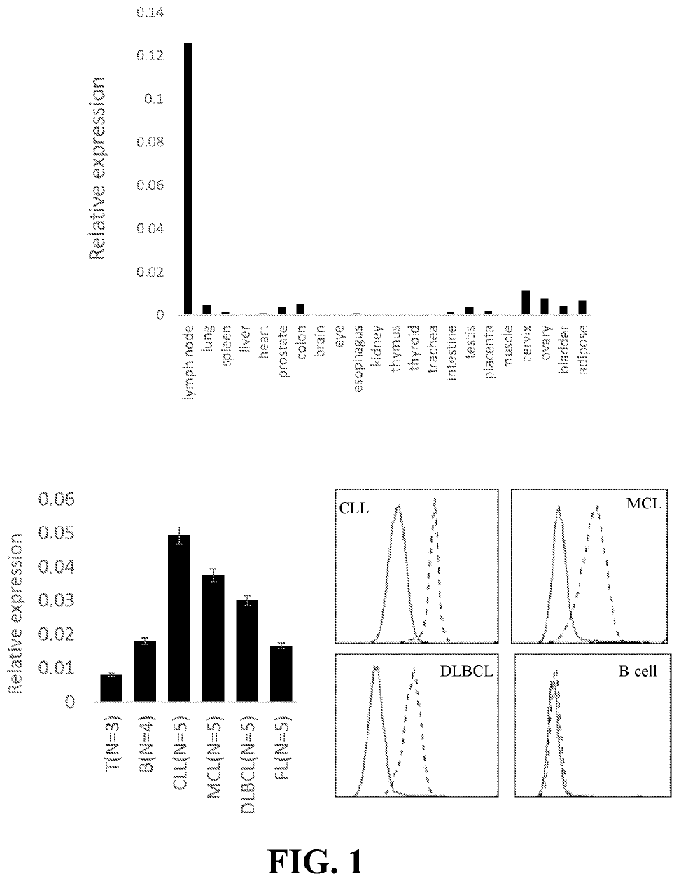

To analyze the expression of ROR1 in normal tissues, realtime PCR assays were performed to quantify ROR1 mRNA in 22 adult normal donor tissues and peripheral blood B and T cells as described in Weng et al., 2012, Blood 120(8):1613-1623. It was found that i) ROR1 expression is restricted to lymph node with highest expression in B cells, and ii) compared with peripheral blood B cells from normal donors, ROR1 was significantly over-expressed in CLL, MCL, and DLBCL (P=0.03) both at transcript (FIG. 1, top and middle panels) and protein level (FIG. 1, bottom panel), which is consistent with prior reports (see, e.g., Barna et al., 2011, Hematological Oncology 29(1):17-21).

### Example 3. Generation and Characterization of Anti-ROR1 Monoclonal Antibodies

To generate anti-ROR1 antibodies, L cells stably transfected with human ROR1 were generated (FIG. 2, top panel). These cells were then used to immunize mice and eight anti-ROR1 monoclonal antibodies (A89, A97, A129, A130, A140, A142, A148, and A183) were generated by standard hybridoma technology.

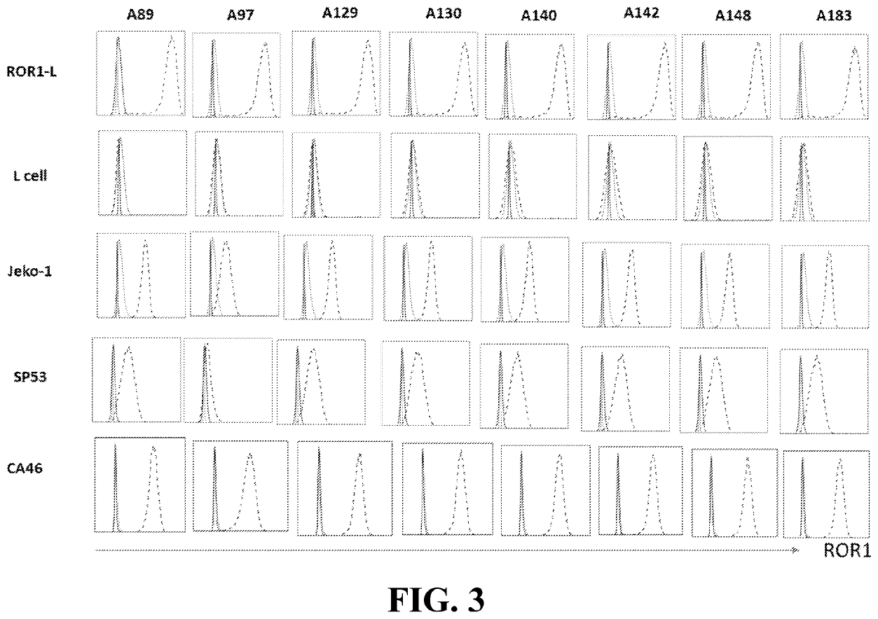

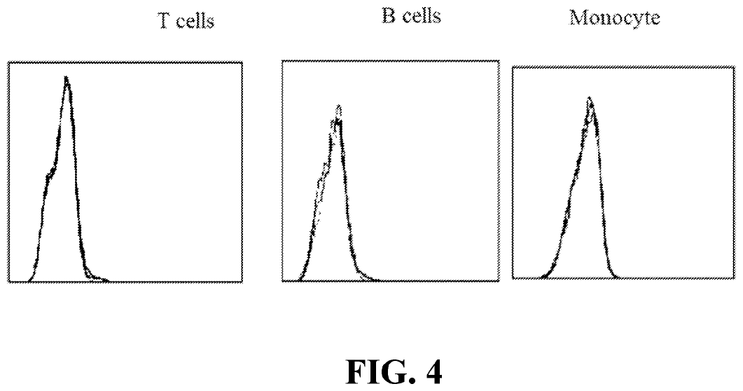

It was found that the eight anti-ROR1 antibodies generated in Example 3 (A89, A97, A129, A130, A140, A142, A148, and A183) specifically bind to human ROR1-expressing L cells but not parental L cells by both ELISA (FIG. 2, bottom panel) and flow cytometry (FIG. 3). The antibody also positively stained human B-cell lymphoma cell lines (Jeko-1, SP-53, and CA46) (FIG. 3), but not human peripheral blood B cells, T cells, or monocytes (FIG. 4).

The CDR sequences of each of the eight anti-ROR1 antibodies were determined using various definition methods. The CDR sequences determined by each definition method for each antibody are represented in Tables 5-12, below.

TABLE 5
A89 CDR sequences.

| Region | Definition | Sequence Fragment | Residues | Length |
|---|---|---|---|---|
| CDR-H1 | Chothia | GYSITSGY (SEQ ID NO: 67) | 26-33 | 8 |
| | AbM | GYSITSGYYWS (SEQ ID NO: 68) | 26-36 | 11 |
| | Kabat | SGYYWS (SEQ ID NO: 69) | 31-36 | 6 |
| | Contact | TSGYYWS (SEQ ID NO: 70) | 30-36 | 7 |
| | IMGT | GYSITSGYY (SEQ ID NO: 12) | 26-34 | 9 |
| CDR-H2 | Chothia | SNDGS (SEQ ID NO: 71) | 53-57 | 5 |
| | AbM | YISNDGSNK (SEQ ID NO: 72) | 51-59 | 9 |
| | Kabat | YISNDGSNKYKPSLKN (SEQ ID NO: 73) | 51-66 | 16 |
| | Contact | WMGYISNDGSNK (SEQ ID NO: 74) | 48-59 | 12 |
| | IMGT | ISNDGSN (SEQ ID NO: 15) | 52-58 | 7 |
| CDR-H3 | Chothia | FPLINYDPWLAY (SEQ ID NO: 75) | 99-110 | 12 |
| | AbM | FPLINYDPWLAY (SEQ ID NO: 75) | 99-110 | 12 |
| | Kabat | FPLINYDPWLAY (SEQ ID NO: 75) | 99-110 | 12 |
| | Contact | ARFPLINYDPWLA (SEQ ID NO: 76) | 97-109 | 13 |
| | IMGT | ARFPLINYDPWLAY (SEQ ID NO: 20) | 97-110 | 14 |
| CDR-L1 | Chothia | RSSQSIVHSNGNTYLE (SEQ ID NO: 77) | 24-39 | 16 |
| | AbM | RSSQSIVHSNGNTYLE (SEQ ID NO: 77) | 24-39 | 16 |
| | Kabat | RSSQSIVHSNGNTYLE (SEQ ID NO: 77) | 24-39 | 16 |
| | Contact | VHSNGNTYLEWY (SEQ ID NO: 78) | 30-41 | 12 |
| | IMGT | QSIVHSNGNTY (SEQ ID NO: 24) | 27-37 | 11 |
| | IMGT | LEWYLQKPGQSPKLLIY (SEQ ID NO: 79) | 38-54 | 17 |
| CDR-L2 | Chothia | KVSNRFS (SEQ ID NO: 80) | 55-61 | 7 |
| | AbM | KVSNRFS (SEQ ID NO: 80) | 55-61 | 7 |
| | Kabat | KVSNRFS (SEQ ID NO: 80) | 55-61 | 7 |
| | Contact | LLIYKVSNRF (SEQ ID NO: 81) | 51-60 | 10 |
| | IMGT | KV (SEQ ID NO: 82) | 55-56 | 2 |
| CDR-L3 | Chothia | FQGSHVPWT (SEQ ID NO: 31) | 94-102 | 9 |
| | AbM | FQGSHVPWT (SEQ ID NO: 31) | 94-102 | 9 |
| | Kabat | FQGSHVPWT (SEQ ID NO: 31) | 94-102 | 9 |
| | Contact | FQGSHVPW (SEQ ID NO: 83) | 94-101 | 8 |
| | IMGT | FQGSHVPWT (SEQ ID NO: 31) | 94-102 | 9 |

TABLE 6
A97 CDR sequences.

| Region | Definition | Sequence Fragment | Residues | Length |
|---|---|---|---|---|
| CDR-H1 | Chothia | GYSFIGY (SEQ ID NO: 84) | 26-32 | 7 |
| | AbM | GYSFIGYSMN (SEQ ID NO: 85) | 26-35 | 10 |
| | Kabat | GYSMN (SEQ ID NO: 86) | 31-35 | 5 |
| | Contact | IGYSMN (SEQ ID NO: 87) | 30-35 | 6 |
| | IMGT | GYSFIGYS (SEQ ID NO: 88) | 26-33 | 8 |
| CDR-H2 | Chothia | DPYYGG (SEQ ID NO: 89) | 52-57 | 6 |
| | AbM | NIDPYYGGTS (SEQ ID NO: 90) | 50-59 | 10 |
| | Kabat | NIDPYYGGTSYNLKFKG (SEQ ID NO: 91) | 50-66 | 17 |
| | Contact | WIGNIDPYYGGTS (SEQ ID NO: 92) | 47-59 | 13 |
| | IMGT | IDPYYGGT (SEQ ID NO: 16) | 51-58 | 8 |
| CDR-H3 | Chothia | LGRGYFDY (SEQ ID NO: 93) | 99-106 | 8 |
| | AbM | LGRGYFDY (SEQ ID NO: 93) | 99-106 | 8 |
| | Kabat | LGRGYFDY (SEQ ID NO: 93) | 99-106 | 8 |
| | Contact | ARLGRGYFD (SEQ ID NO: 94) | 97-105 | 9 |
| | IMGT | ARLGRGYFDY (SEQ ID NO: 21) | 97-106 | 10 |
| CDR-L1 | Chothia | RASQSITNNLH (SEQ ID NO: 95) | 24-34 | 11 |
| | AbM | RASQSITNNLH (SEQ ID NO: 95) | 24-34 | 11 |
| | Kabat | RASQSITNNLH (SEQ ID NO: 95) | 24-34 | 11 |
| | Contact | TNNLHWY (SEQ ID NO: 96) | 30-36 | 7 |
| | IMGT | QSITNN (SEQ ID NO: 97) | 27-32 | 6 |
| CDR-L2 | Chothia | SASQSIS (SEQ ID NO: 98) | 50-56 | 7 |
| | AbM | SASQSIS (SEQ ID NO: 98) | 50-56 | 7 |
| | Kabat | SASQSIS (SEQ ID NO: 98) | 50-56 | 7 |
| | Contact | LLIKSASQSI (SEQ ID NO: 99) | 46-55 | 10 |
| | IMGT | SA (SEQ ID NO: 100) | 50-51 | 2 |
| CDR-L3 | Chothia | QQSNSWPHT (SEQ ID NO: 32) | 89-97 | 9 |
| | AbM | QQSNSWPHT (SEQ ID NO: 32) | 89-97 | 9 |
| | Kabat | QQSNSWPHT (SEQ ID NO: 32) | 89-97 | 9 |
| | Contact | QQSNSWPH (SEQ ID NO: 101) | 89-96 | 8 |
| | IMGT | QQSNSWPHT (SEQ ID NO: 32) | 89-97 | 9 |

TABLE 7
A129 CDR sequences.

| Region | Definition | Sequence Fragment | Residues | Length |
|---|---|---|---|---|
| CDR-H1 | Chothia | GYSITSGY (SEQ ID NO: 67) | 26-33 | 8 |
| | AbM | GYSITSGYYWN (SEQ ID NO: 102) | 26-36 | 11 |
| | Kabat | SGYYWN (SEQ ID NO: 103) | 31-36 | 6 |
| | Contact | TSGYYWN (SEQ ID NO: 104) | 30-36 | 7 |
| | IMGT | GYSITSGYY (SEQ ID NO: 12) | 26-34 | 9 |
| CDR-H2 | Chothia | RNDGS (SEQ ID NO: 105) | 53-57 | 5 |
| | AbM | YIRNDGSNN (SEQ ID NO: 106) | 51-59 | 9 |
| | Kabat | YIRNDGSNNYNPSLKN (SEQ ID NO: 107) | 51-66 | 16 |
| | Contact | WMGYIRNDGSNN (SEQ ID NO: 108) | 48-59 | 12 |
| | IMGT | IRNDGSN (SEQ ID NO: 17) | 52-58 | 7 |
| CDR-H3 | Chothia | FPLINYDPWFAY (SEQ ID NO: 109) | 99-110 | 12 |
| | AbM | FPLINYDPWFAY (SEQ ID NO: 109) | 99-110 | 12 |
| | Kabat | FPLINYDPWFAY (SEQ ID NO: 109) | 99-110 | 12 |
| | Contact | ARFPLINYDPWFA (SEQ ID NO: 110) | 97-109 | 13 |
| | IMGT | ARFPLINYDPWFAY (SEQ ID NO: 22) | 97-110 | 14 |
| CDR-L1 | Chothia | RSSQNVVHRNGNTYLE (SEQ ID NO: 111) | 24-39 | 16 |
| | AbM | RSSQNVVHRNGNTYLE (SEQ ID NO: 111) | 24-39 | 16 |
| | Kabat | RSSQNVVHRNGNTYLE (SEQ ID NO: 111) | 24-39 | 16 |
| | Contact | VHRNGNTYLEWY (SEQ ID NO: 112) | 30-41 | 12 |
| | IMGT | QNVVHRNGNTY (SEQ ID NO: 26) | 27-37 | 11 |
| CDR-L2 | Chothia | KVSNRFS (SEQ ID NO: 80) | 55-61 | 7 |
| | AbM | KVSNRFS (SEQ ID NO: 80) | 55-61 | 7 |
| | Kabat | KVSNRFS (SEQ ID NO: 80) | 55-61 | 7 |
| | Contact | LLIYKVSNRF (SEQ ID NO: 81) | 51-60 | 10 |
| | IMGT | KV (SEQ ID NO: 82) | 55-56 | 2 |
| CDR-L3 | Chothia | FQGSHVPWT (SEQ ID NO: 31) | 94-102 | 9 |
| | AbM | FQGSHVPWT (SEQ ID NO: 31) | 94-102 | 9 |
| | Kabat | FQGSHVPWT (SEQ ID NO: 31) | 94-102 | 9 |
| | Contact | FQGSHVPW (SEQ ID NO: 83) | 94-101 | 8 |
| | IMGT | FQGSHVPWT (SEQ ID NO: 31) | 94-102 | 9 |

TABLE 8
A130 CDR sequences.

| Region | Definition | Sequence Fragment | Residues | Length |
|---|---|---|---|---|
| CDR-H1 | Chothia | GYSITSGY (SEQ ID NO: 67) | 26-33 | 8 |
| | AbM | GYSITSGYYWN (SEQ ID NO: 102) | 26-36 | 11 |
| | Kabat | SGYYWN (SEQ ID NO: 103) | 31-36 | 6 |
| | Contact | TSGYYWN (SEQ ID NO: 104) | 30-36 | 7 |
| | IMGT | GYSITSGYY (SEQ ID NO: 12) | 26-34 | 9 |
| CDR-H2 | Chothia | RNDGS (SEQ ID NO: 105) | 53-57 | 5 |
| | AbM | YIRNDGSNN (SEQ ID NO: 106) | 51-59 | 9 |
| | Kabat | YIRNDGSNNYNPSLKN (SEQ ID NO: 107) | 51-66 | 16 |
| | Contact | WMGYIRNDGSNN (SEQ ID NO: 108) | 48-59 | 12 |
| | IMGT | IRNDGSN (SEQ ID NO: 17) | 52-58 | 7 |
| CDR-H3 | Chothia | FPLINYDPWFAY (SEQ ID NO: 109) | 99-110 | 12 |
| | AbM | FPLINYDPWFAY (SEQ ID NO: 109) | 99-110 | 12 |
| | Kabat | FPLINYDPWFAY (SEQ ID NO: 109) | 99-110 | 12 |
| | Contact | ARFPLINYDPWFA (SEQ ID NO: 110) | 97-109 | 13 |
| | IMGT | ARFPLINYDPWFAY (SEQ ID NO: 22) | 97-110 | 14 |
| CDR-L1 | Chothia | ITSTDIDDDMN (SEQ ID NO: 113) | 24-34 | 11 |
| | AbM | ITSTDIDDDMN (SEQ ID NO: 113) | 24-34 | 11 |
| | Kabat | ITSTDIDDDMN (SEQ ID NO: 113) | 24-34 | 11 |
| | Contact | DDDMNWY (SEQ ID NO: 114) | 30-36 | 7 |
| | IMGT | TDIDDD (SEQ ID NO: 27 | 27-32 | 6 |
| CDR-L2 | Chothia | EANTLRP (SEQ ID NO: 115) | 50-56 | 7 |
| | AbM | EANTLRP (SEQ ID NO: 115) | 50-56 | 7 |
| | Kabat | EANTLRP (SEQ ID NO: 115) | 50-56 | 7 |
| | Contact | LLISEANTLR (SEQ ID NO: 116) | 46-55 | 10 |
| | IMGT | EA (SEQ ID NO: 117) | 50-51 | 2 |
| CDR-L3 | Chothia | LQSYNMPLT (SEQ ID NO: 33) | 89-97 | 9 |
| | AbM | LQSYNMPLT (SEQ ID NO: 33) | 89-97 | 9 |
| | Kabat | LQSYNMPLT (SEQ ID NO: 33) | 89-97 | 9 |
| | Contact | LQSYNMPL (SEQ ID NO: 118) | 89-96 | 8 |
| | IMGT | LQSYNMPLT (SEQ ID NO: 33) | 89-97 | 9 |

TABLE 9
A140 CDR sequences.

| Region | Definition | Sequence Fragment | Residues | Length |
|---|---|---|---|---|
| CDR-H1 | Chothia | GYSITSGY (SEQ ID NO: 67) | 26-33 | 8 |
| | AbM | GYSITSGYYWN (SEQ ID NO: 102) | 26-36 | 11 |
| | Kabat | SGYYWN (SEQ ID NO: 103) | 31-36 | 6 |
| | Contact | TSGYYWN (SEQ ID NO: 104) | 30-36 | 7 |
| | IMGT | GYSITSGYY (SEQ ID NO: 12) | 26-34 | 9 |
| CDR-H2 | Chothia | SNDGN (SEQ ID NO: 119) | 53-57 | 5 |
| | AbM | YISNDGNNK (SEQ ID NO: 120) | 51-59 | 9 |
| | Kabat | YISNDGNNKYKPSLKN (SEQ ID NO: 121) | 51-66 | 16 |
| | Contact | WMGYISNDGNNK (SEQ ID NO: 122) | 48-59 | 12 |
| | IMGT | ISNDGNN (SEQ ID NO: 18) | 52-58 | 7 |
| CDR-H3 | Chothia | FPLINYDPWLAY (SEQ ID NO: 75) | 99-110 | 12 |
| | AbM | FPLINYDPWLAY (SEQ ID NO: 75) | 99-110 | 12 |
| | Kabat | FPLINYDPWLAY (SEQ ID NO: 75) | 99-110 | 12 |
| | Contact | ARFPLINYDPWLA (SEQ ID NO: 76) | 97-109 | 13 |
| | IMGT | ARFPLINYDPWLAY (SEQ ID NO: 20) | 97-110 | 14 |
| CDR-L1 | Chothia | RSSQSIVHSNGNTYLE (SEQ ID NO: 77) | 24-39 | 16 |
| | AbM | RSSQSIVHSNGNTYLE (SEQ ID NO: 77) | 24-39 | 16 |
| | Kabat | RSSQSIVHSNGNTYLE (SEQ ID NO: 77) | 24-39 | 16 |
| | Contact | VHSNGNTYLEWY (SEQ ID NO: 78) | 30-41 | 12 |
| | IMGT | QSIVHSNGNTY (SEQ ID NO: 24) | 27-37 | 11 |
| CDR-L2 | Chothia | KVSNRFS (SEQ ID NO: 80) | 55-61 | 7 |
| | AbM | KVSNRFS (SEQ ID NO: 80) | 55-61 | 7 |
| | Kabat | KVSNRFS (SEQ ID NO: 80) | 55-61 | 7 |
| | Contact | LLIYKVSNRF (SEQ ID NO: 81) | 51-60 | 10 |
| | IMGT | KV (SEQ ID NO: 82) | 55-56 | 2 |
| CDR-L3 | Chothia | FQGSHVPWT (SEQ ID NO: 31) | 94-102 | 9 |
| | AbM | FQGSHVPWT (SEQ ID NO: 31) | 94-102 | 9 |
| | Kabat | FQGSHVPWT (SEQ ID NO: 31) | 94-102 | 9 |
| | Contact | FQGSHVPW (SEQ ID NO: 83 | 94-101 | 8 |
| | IMGT | FQGSHVPWT (SEQ ID NO: 31) | 94-102 | 9 |

TABLE 10
A142 CDR sequences.

| Region | Definition | Sequence Fragment | Residues | Length |
|---|---|---|---|---|
| CDR-H1 | Chothia | GYSFTGY (SEQ ID NO: 123) | 26-32 | 7 |
| | AbM | GYSFTGYYMH (SEQ ID NO: 124) | 26-35 | 10 |
| | Kabat | GYYMH (SEQ ID NO: 125) | 31-35 | 5 |
| | Contact | TGYYMH (SEQ ID NO: 126) | 30-35 | 6 |
| | IMGT | GYSFTGYY (SEQ ID NO: 14) | 26-33 | 8 |
| CDR-H2 | Chothia | YPNNGG (SEQ ID NO: 127) | 52-57 | 6 |
| | AbM | RVYPNNGGTS (SEQ ID NO: 128) | 50-59 | 10 |
| | Kabat | RVYPNNGGTSYNQKFKG (SEQ ID NO: 129) | 50-66 | 17 |
| | Contact | WIGR VYPNNGGTS (SEQ ID NO: 130) | 47-59 | 13 |
| | IMGT | VYPNNGGT (SEQ ID NO: 19) | 51-58 | 8 |
| CDR-H3 | Chothia | IHYYGFYSMDY (SEQ ID NO: 20) | 99-109 | 11 |
| | AbM | IHYYGFYSMDY (SEQ ID NO: 20) | 99-109 | 11 |
| | Kabat | IHYYGFYSMDY (SEQ ID NO: 20) | 99-109 | 11 |
| | Contact | ALIHYYGFYSMD (SEQ ID NO: 131) | 97-108 | 12 |
| | IMGT | ALIHYYGFYSMDY (SEQ ID NO: 23) | 97-109 | 13 |
| CDR-L1 | Chothia | RSSQSIVHSNGNTYLE (SEQ ID NO: 77) | 24-39 | 16 |
| | AbM | RSSQSIVHSNGNTYLE (SEQ ID NO: 77) | 24-39 | 16 |
| | Kabat | RSSQSIVHSNGNTYLE (SEQ ID NO: 77) | 24-39 | 16 |
| | Contact | VHSNGNTYLEWY (SEQ ID NO: 78) | 30-41 | 12 |
| | IMGT | QSIVHSNGNTY (SEQ ID NO: 24) | 27-37 | 11 |
| CDR-L2 | Chothia | KVSNRFS (SEQ ID NO: 80) | 55-61 | 7 |
| | AbM | KVSNRFS (SEQ ID NO: 80) | 55-61 | 7 |
| | Kabat | KVSNRFS (SEQ ID NO: 80) | 55-61 | 7 |
| | Contact | LLIYKVSNRF (SEQ ID NO: 81) | 51-60 | 10 |
| | IMGT | KV (SEQ ID NO: 82) | 55-56 | 2 |
| CDR-L3 | Chothia | FQGSHVPWT (SEQ ID NO: 31) | 94-102 | 9 |
| | AbM | FQGSHVPWT (SEQ ID NO: 31) | 94-102 | 9 |
| | Kabat | FQGSHVPWT (SEQ ID NO: 31) | 94-102 | 9 |
| | Contact | FQGSHVPW (SEQ ID NO: 83) | 94-101 | 8 |
| | IMGT | FQGSHVPWT (SEQ ID NO: 31) | 94-102 | 9 |

TABLE 11
A148 CDR sequences.

| Region | Definition | Sequence Fragment | Residues | Length |
|---|---|---|---|---|
| CDR-H1 | Chothia | GYSITSGY (SEQ ID NO: 67) | 26-33 | 8 |
| | AbM | GYSITSGYYWN (SEQ ID NO: 102) | 26-36 | 11 |
| | Kabat | SGYYWN (SEQ ID NO: 103) | 31-36 | 6 |
| | Contact | TSGYYWN (SEQ ID NO: 104) | 30-36 | 7 |
| | IMGT | GYSITSGYY (SEQ ID NO: 12) | 26-34 | 9 |
| CDR-H2 | Chothia | SNDGS (SEQ ID NO: 71) | 53-57 | 5 |
| | AbM | YISNDGSNK (SEQ ID NO: 72) | 51-59 | 9 |
| | Kabat | YISNDGSNKYKPSLKN (SEQ ID NO: 73) | 51-66 | 16 |
| | Contact | WMGYISNDGSNK (SEQ ID NO: 74) | 48-59 | 12 |
| | IMGT | ISNDGSN (SEQ ID NO: 15) | 52-58 | 7 |
| CDR-H3 | Chothia | FPLINYDPWLAY (SEQ ID NO: 75) | 99-110 | 12 |
| | AbM | FPLINYDPWLAY (SEQ ID NO: 75) | 99-110 | 12 |
| | Kabat | FPLINYDPWLAY (SEQ ID NO: 75) | 99-110 | 12 |
| | Contact | ARFPLINYDPWLA (SEQ ID NO: 76) | 97-109 | 13 |
| | IMGT | ARFPLINYDPWLAY (SEQ ID NO: 20) | 97-110 | 14 |
| CDR-L1 | Chothia | RSSQNVVHRNGNTYLE (SEQ ID NO: 111) | 24-39 | 16 |
| | AbM | RSSQNVVHRNGNTYLE (SEQ ID NO: 111) | 24-39 | 16 |
| | Kabat | RSSQNVVHRNGNTYLE (SEQ ID NO: 111) | 24-39 | 16 |
| | Contact | VHRNGNTYLEWY (SEQ ID NO: 112) | 30-41 | 12 |
| | IMGT | QNVVHRNGNTY (SEQ ID NO: 26) | 27-37 | 11 |
| CDR-L2 | Chothia | KVSNRFS (SEQ ID NO: 80) | 55-61 | 7 |
| | AbM | KVSNRFS (SEQ ID NO: 80) | 55-61 | 7 |
| | Kabat | KVSNRFS (SEQ ID NO: 80) | 55-61 | 7 |
| | Contact | LLIYKVSNRF (SEQ ID NO: 81) | 51-60 | 10 |
| | IMGT | KV (SEQ ID NO: 82) | 55-56 | 2 |
| CDR-L3 | Chothia | FQGSHVPWT (SEQ ID NO: 31) | 94-102 | 9 |
| | AbM | FQGSHVPWT (SEQ ID NO: 31) | 94-102 | 9 |
| | Kabat | FQGSHVPWT (SEQ ID NO: 31) | 94-102 | 9 |
| | Contact | FQGSHVPW (SEQ ID NO: 83) | 94-101 | 8 |
| | IMGT | FQGSHVPWT (SEQ ID NO: 31) | 94-102 | 9 |

TABLE 12
A183 CDR sequences.

| Region | Definition | Sequence Fragment | Residues | Length |
|---|---|---|---|---|
| CDR-H1 | Chothia | GYSITSGY (SEQ ID NO: 67) | 26-33 | 8 |
| | AbM | GYSITSGYYWS (SEQ ID NO: 68) | 26-36 | 11 |
| | Kabat | SGYYWS (SEQ ID NO: 69) | 31-36 | 6 |
| | Contact | TSGYYWS (SEQ ID NO: 70) | 30-36 | 7 |
| | IMGT | GYSITSGYY (SEQ ID NO: 12) | 26-34 | 9 |
| CDR-H2 | Chothia | SNDGS (SEQ ID NO: 71) | 53-57 | 5 |
| | AbM | YISNDGSNK (SEQ ID NO: 72) | 51-59 | 9 |
| | Kabat | YISNDGSNKYKPSLKN (SEQ ID NO: 73) | 51-66 | 16 |
| | Contact | WMGYISNDGSNK (SEQ ID NO: 74) | 48-59 | 12 |
| | IMGT | ISNDGSN (SEQ ID NO: 15) | 52-58 | 7 |
| CDR-H3 | Chothia | FPLINYDPWLAY (SEQ ID NO: 75) | 99-110 | 12 |
| | AbM | FPLINYDPWLAY (SEQ ID NO: 75) | 99-110 | 12 |
| | Kabat | FPLINYDPWLAY (SEQ ID NO: 75) | 99-110 | 12 |
| | Contact | ARFPLINYDPWLA (SEQ ID NO: 76) | 97-109 | 13 |
| | IMGT | ARFPLINYDPWLAY (SEQ ID NO: 20) | 97-110 | 14 |
| CDR-L1 | Chothia | RSSQNVVHRNGNTYLE (SEQ ID NO: 111) | 24-39 | 16 |
| | AbM | RSSQNVVHRNGNTYLE (SEQ ID NO: 111) | 24-39 | 16 |
| | Kabat | RSSQNVVHRNGNTYLE (SEQ ID NO: 111) | 24-39 | 16 |
| | Contact | VHRNGNTYLEWY (SEQ ID NO: 112) | 30-41 | 12 |
| | IMGT | QNVVHRNGNTY (SEQ ID NO: 26) | 27-37 | 11 |
| CDR-L2 | Chothia | KVSNRFS (SEQ ID NO: 80) | 55-61 | 7 |
| | AbM | KVSNRFS (SEQ ID NO: 80) | 55-61 | 7 |
| | Kabat | KVSNRFS (SEQ ID NO: 80) | 55-61 | 7 |
| | Contact | LLIYKVSNRF (SEQ ID NO: 81) | 51-60 | 10 |
| | IMGT | KV (SEQ ID NO: 82) | 55-56 | 2 |
| CDR-L3 | Chothia | FQGSHVPWT (SEQ ID NO: 31) | 94-102 | 9 |
| | AbM | FQGSHVPWT (SEQ ID NO: 31) | 94-102 | 9 |
| | Kabat | FQGSHVPWT (SEQ ID NO: 31) | 94-102 | 9 |
| | Contact | FQGSHVPW (SEQ ID NO: 83) | 94-101 | 8 |
| | IMGT | FQGSHVPWT (SEQ ID NO: 31) | 94-102 | 9 |

### Example 4. Generation and Characterization of ROR1-Specific CAR T Cells

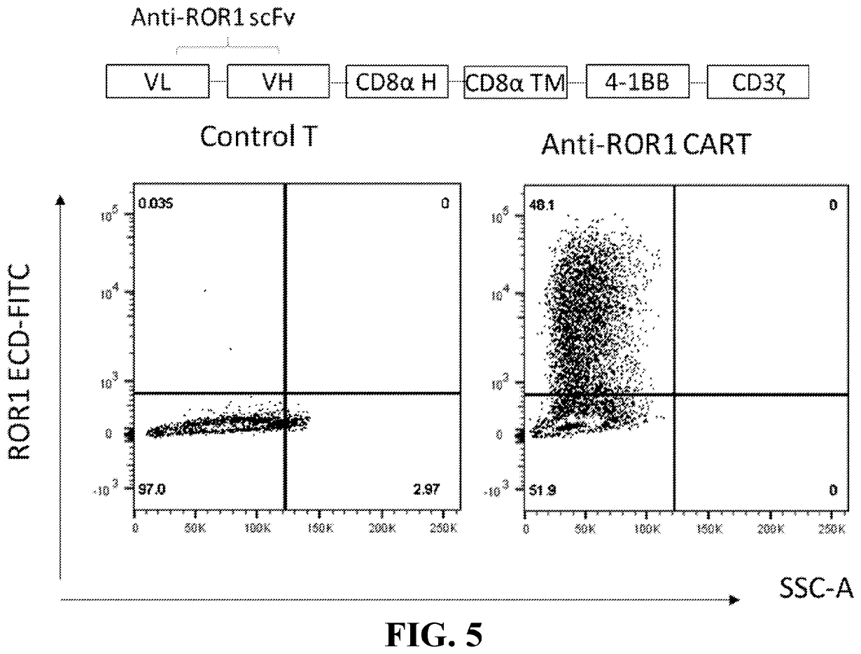

Healthy donor T cells were lentivirally transduced with ROR1-specific CAR (A129-8a-BB-z) containing anti-ROR1 scFv (clone A129), CD8α hinge, CD8α transmembrane, and 4-1BB and CD3ζ signaling domains (FIG. 5, top panel; A129-8α-BB-ζ in Table 13). Flow cytometric analysis for surface expression of anti-ROR1 CAR using FITC-conjugated ROR1 extracellular domain protein staining revealed high transduction efficiency after 72 hours (FIG. 5, bottom panel).

In co-culture assays, anti-ROR1 CAR T cells induced significant lysis of ROR1-transduced L cells and ROR1+ B-cell lymphoma cell line cells (Jeko-1, sp53, and CA46), but not parental L cells or ROR1-negative NK-cell leukemia cell line cells (NK92) (FIGS. 6A-6B).

Further experiments were conducted with additional anti-ROR1 CAR-T cells having anti-ROR1 scFv (clone A129) and different hinge and transmembrane domains (CD8α or CD28) and costimulatory domains (CD28, 4-1BB, or OX40). See Table 13 below. The CAR-T cells were co-cultured with green fluorescent protein (GFP)-transduced, ROR1-expressing B-cell lymphoma cell lines, CA46, Jeko-1, and SP53 at an Effector:Target (E:T) ratio of 1:1. I

TABLE 13
Anti-ROR1 CAR-T cells construct configurations.

| | | SEQ ID NO |
|---|---|---|
| Construct | Configuration | (DNA/AA) |
| A129-8α-BB-ζ | Anti-ROR1 scFv | 50/51 |
| | (A129)/CD8α/4-1BB-CD3ζ | |
| A129-8α-OX40-ζ | Anti-ROR1/CD8α/OX40-CD3ζ | 59/60 |
| A129-28-28-ζ | Anti-ROR1/CD28/CD28-CD3ζ | 61/62 |
| A129-28-OX40-ζ | Anti-ROR1/CD28/OX40-CD3ζ | 63/64 |
| A129-28-BB-ζ | Anti-ROR1/CD28/4-1BB-CD3ζ | 65/66 |

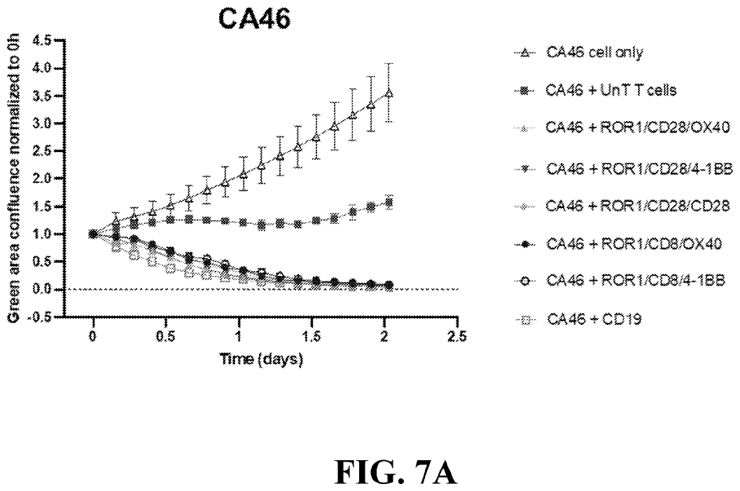

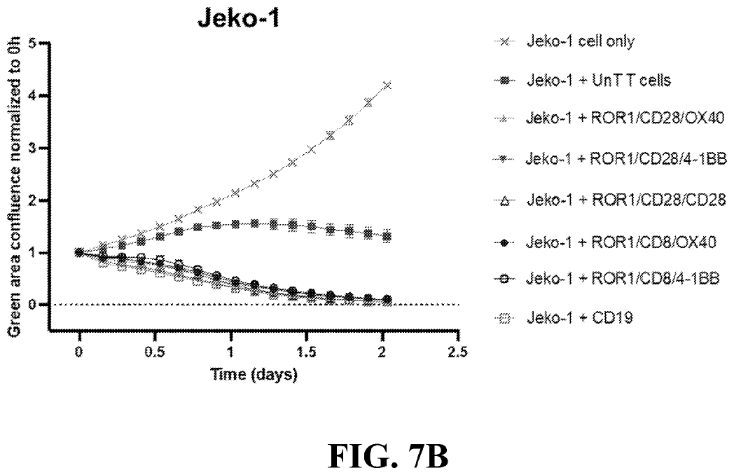

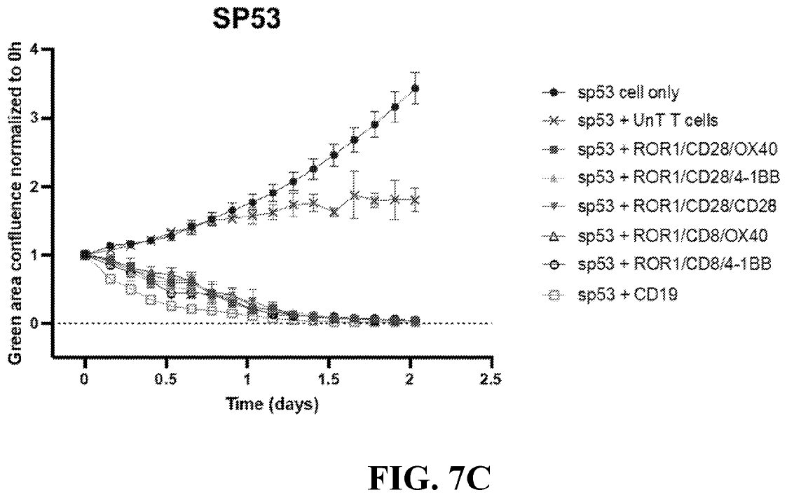

The cytotoxic activity of the CAR-T cells was assessed by determining change in GFP-positive cells (green area confluence) by Incucyte live cell imaging serially over time (FIGS. 7A-7C). Anti-ROR1 CAR-T cells induced significant lysis of ROR1+ B-cell lymphoma lines compared to tumor alone and untransduced T cell (UnT) groups (p<0.05). Anti-CD19 CAR-T cells (CD19) were used as positive control. The experiments were performed in triplicate wells.

### Example 5. Long-Term Cytotoxicity of Anti-ROR1 CAR-T Cells Against B-Cell Lymphoma Cells

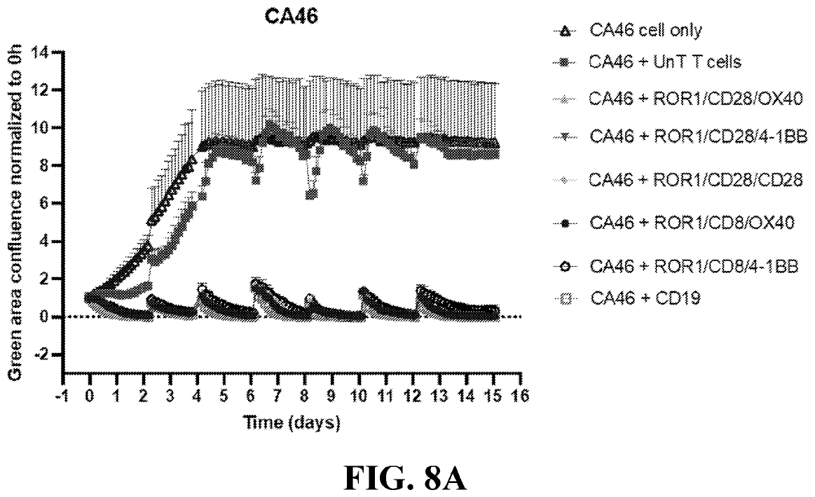

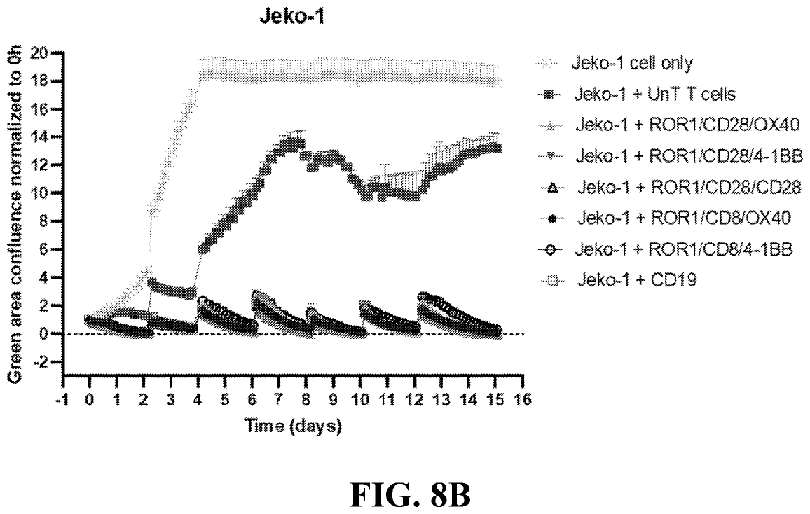

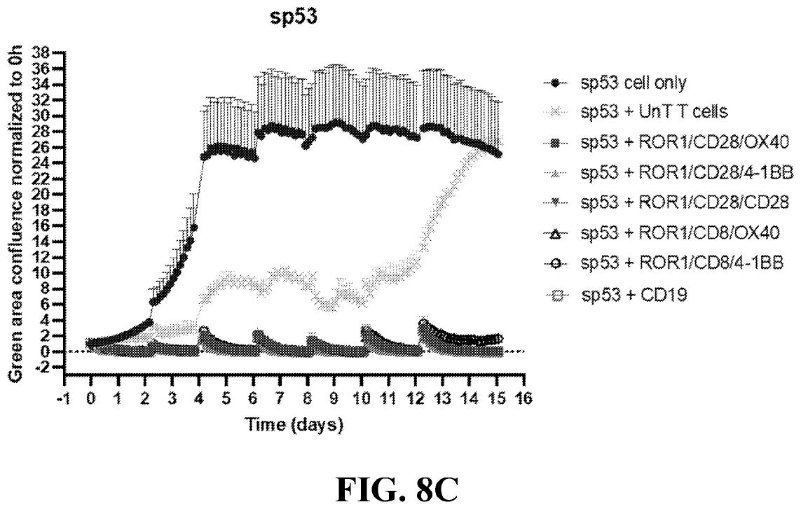

Anti-ROR1 CAR-T cells with different hinge and transmembrane domains (CD8α or CD28) and costimulatory domains (CD28, 4-1BB, or OX40) (see Table 13) were co-cultured with green fluorescent protein (GFP)-transduced, ROR1-expressing B-cell lymphoma cell lines, CA46, Jeko-1, and SP53 at an Effector:Target (E:T) ratio of 1:1. Fresh batches of tumor cells were added to the wells every 2 days and the cytotoxic activity of the CAR-T cells was assessed by determining change in GFP-positive cells (green area confluence) by INCUCYTE® live cell imaging serially over time for 15 days (15d) (FIGS. 8A-8C). Anti-ROR1 CAR-T cells exhibited significant cytotoxic activity against ROR1+ B-cell lymphoma cell lines long-term for over 2 weeks compared to tumor alone and untransduced T cell (UnT) groups (p<0.05). Anti-CD19 CAR-T cells (CD19) were used as positive control. The experiments were performed in triplicate wells.

### Example 6. Antitumor Effects of Multiple Anti-ROR1 Constructs Against Jeko-1 B-Cell Lymphoma Cells In Vivo

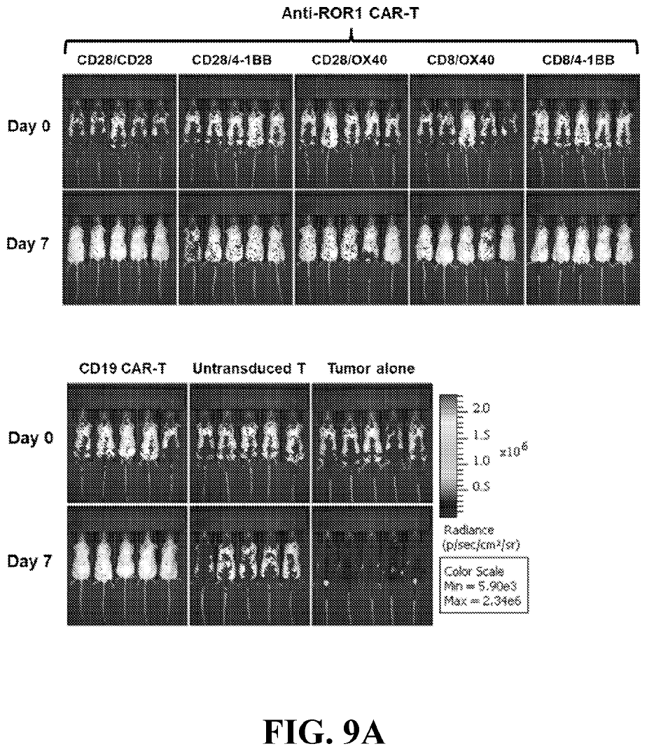

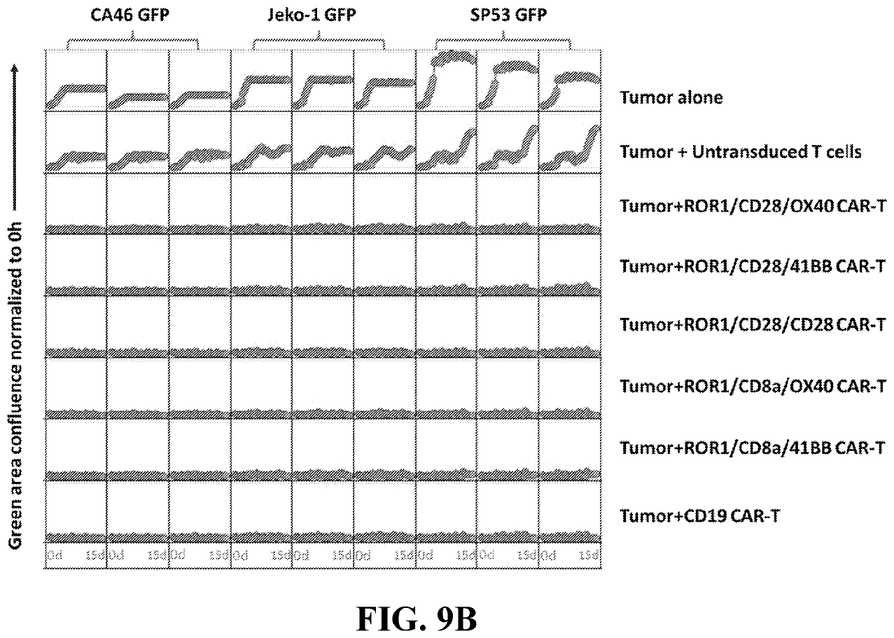

Luciferase-transduced ROR1+ Jeko-1 mantle cell lymphoma tumor cells were injected via tail vein into NSG mice (0.3×10⁶ cells/mouse) on day −7. On day 0, 5×10⁶ anti-ROR1 CAR-T cells with different hinge and transmembrane domains (CD8α or CD28) and costimulatory domains (CD28, 4-1BB, or OX40) were injected via tail vein. Tumor burden was determined by bioluminescent imaging at the indicated time points (FIGS. 9A-9B). Anti-ROR1 CAR-T cells induced rapid elimination of tumor cells compared to tumor alone and untransduced T cell groups by day 7 (FIGS. 9A-9B). Anti-CD19 CAR-T cells were used as positive control.

List of sequences identified in this disclosure:

SEQ ID NO: 34:
A89 VH nucleotide sequence
GACGTGCAGCTGCAGGAGAGCGGTCCTGGCCTGGTGAAGCCTAGCCAATC
TCTGAGCCTGACCTGCAGCGTGTCCGGATATTCCATCACCAGCGGCTATT
ACTGGTCCTGGATCAGACAGTTTCCAGGCAACAAGCTGGAGTGGATGGGC
TACATCAGCAACGACGGCAGCAACAAGTACAAGCCCAGCCTGAAAAACAG
AATCAGTATTACAAGAGATACATCTAAGAACCAGTTCTTCCTGAAGCTTA
AAAGCGTGACAACCGAGGACACCGCCACCTACTACTGCGCCCGGTTCCCT
CTGATCAACTACGACCCCTGGCTGGCCTACTGGGGCCAGGGCACACTGGT
TACAGTGTCCGCC
SEQ ID NO: 35:
A89 VL nucleotide sequence
GATGTCCTGATGACCCAGACCCCTCTCAGCCTGCCAGTGTCCCTGGGCGA
CCAGGCCAGCATCAGCTGCAGAAGCAGCCAGTCCATCGTGCATAGCAATG
GAAATACCTACCTGGAATGGTATCTGCAAAAGCCTGGACAGAGCCCTAAG
CTGCTGATCTACAAAGTGAGCAACCGGTTCAGCGGAGTGCCCGACAGATT
CTCTGGCTCTGGCTCTGGCACAGATTTCACCCTGAAGATCTCTAGGGTGG
AAGCCGAGGACCTGGGCGTGTACTACTGTTTTCAGGGCAGCCACGTCCCT
TGGACCTTCGGCGGCGGCACCAAGCTGGAAATCAAC
SEQ ID NO: 36:
A97 VH nucleotide sequence
CAGTTGCAGCAGAGCGGTCCTGAGCTGGAAAAGCCAGGCGCCAGCGTGAA
GATCAGCTGTAAAGCTTCTGGCTATAGCTTCATCGGCTACAGCATGAACT
GGGTGCGGCAGAGCGATGGCAAGTCTCTGGAATGGATTGGCAACATCGAC
CCTTACTACGGCGGTACAAGCTACAATCTGAAATTCAAGGGCAAGGCCAC
CCTGACAGTGGACAAGTCTAGCAGCACCGCCTACATGCAGCTGAAGAGCC
TGACCTCCGAGGATAGCGCCGTCTACTACTGCGCCAGACTGGGCAGAGGA
TATTTCGACTACTGGGGCCAGGGCACCACCCTGACCGTGTCCAGC
SEQ ID NO: 37:
A97 VL nucleotide sequence
GATATCGTGCTGACCCAGTCTCCTGCTACACTGAGCGTTACACCAGGCGA
CTCCGTGTCTCTCAGTTGCAGAGCCAGCCAGTCCATCACAAACAACCTGC
ATTGGTATCAGCAAAAGAGCCACGAAAGCCCCAGACTGCTGATCAAGAGC
GCCAGCCAGAGCATCAGCGGCATCCCTAGCCGGTTCAGCGGCTCTGGCAG
CGGAACCGACTTCACCCTTTCCATCAATTCTGTGGAGACCGAGGACTTCG
GCATGTACTTTTGCCAGCAAAGCAACAGCTGGCCCCACACCTTTGGAGGC
GGAACAAAACTGGAAATCAAGGGCGGA
SEQ ID NO: 38:
A129 VH nucleotide sequence
GACGTGCAGCTGCAGGAGAGCGGACCTGGACTGGTTAAGCCTAGCCAAAG
CCTGAGCCTGACCTGCAGCGTGACAGGCTACAGCATCACCAGCGGGTATT
ACTGGAACTGGATCAGACAGTTCCCCGGCAACAAGCTGGAATGGATGGGC
TACATCAGAAACGACGGCTCCAACAACTACAACCCTTCTCTGAAAAACCG
GATCTCTATTACCAGAGATACCTCCAAGAACCAGTTTTTCCTGAAGCTGA
ATAGCGTCACGACCGAGGACACAGCTACATACTATTGTGCTCGGTTTCCA
CTGATCAACTACGACCCCTGGTTCGCCTACTGGGGCCAGGGCACCCTGGT
GACGGTGTCCGCC
SEQ ID NO: 39:
A129 VL nucleotide sequence
GACGTGCTGATGACACAGGCCCCTCTGTCTCTGCCCGTCAGCCTCGGCGA
CCAGGCCAGCATCAGCTGCAGAAGCAGCCAGAACGTGGTGCATAGAAATG
GCAATACCTACCTGGAATGGTATCTGCAGAAACCTGGCCAATCTCCAAAG
CTGCTGATCTACAAGGTGTCCAACAGATTCAGCGGCGTGCCTGATAGATT
CAGCGGCAGCGGATCTGGAACAGACTTCACCCTGAAGATCAGCAGAGTGG
AAGCCGAGGATCTGGGCGTGTACTACTGCTTCCAGGGCAGCCACGTGCCC
TGGACCTTCGGCGGCGGCACCAAGCTCGAGATCAAG
SEQ ID NO: 40:
A130 VH nucleotide sequence
GACGTGCAGCTGCAGGAGAGCGGCCCTGGCCTGGTTAAGCCCTCTCAGAG
CCTTTCTCTGACCTGCAGCGTGACCGGATACAGCATCACCAGCGGCTATT
ACTGGAACTGGATCAGACAGTTTCCAGGCAACAAGCTCGAGTGGATGGGC
TATATCCGGAACGACGGCAGCAACAACTACAACCCAAGCCTGAAAAATAG
AATCAGCATCACCCGGGACACCAGCAAGAACCAGTTCTTCCTGAAGCTGA
ACAGCGTGACCACGGAAGATACCGCCACCTACTACTGTGCTAGATTCCCC
CTGATTAACTACGACCCTTGGTTCGCCTACTGGGGCCAGGGCACACTGGT
CACCGTGTCCGCC
SEQ ID NO: 41:
A130 VL nucleotide sequence
GAAACCACAGTGACACAAAGCCCTGCCAGCCTGTCCGTGGCTACAGGAGA
GAAGGTGACCATCCGGTGCATCACCAGTACAGATATCGACGACGATATGA
ACTGGTATCAGCAGAAGCCTGGAGAACCCCCCAAGCTGCTGATCTCCGAG
GCCAATACCCTGAGACCCGGCGTGCCTTCTAGGTTCAGCAGCTCTGGTTA
CGGCACAGACTTCGTGTTCACCATCGAGAACACACTGTCTGAGGATGTGG
CCGACTACTACTGCCTGCAAAGCTACAATATGCCTCTGACCTTTGGCGCC
GGCACAACCCTGGAACTGAAA
SEQ ID NO: 42:
A140 VH nucleotide sequence
GACGTGCAGCTGCAGGAGAGCGGACCTGGCCTGGTGAAGCCTAGCCAGAG
CCTGAGCCTGACATGTTCCGTGAGCGGCTACAGCATCACCAGCGGCTACT
ACTGGAACTGGATTAGACAGTTTCCCGGCAACAAGCTGGAGTGGATGGGA
TACATCAGCAACGACGGCAACAACAAATACAAGCCAAGCCTGAAAAACCG
GATCTCTATCACAAGAGATACCAGCAAGAACCAGTTTTTCCTGAAGCTGA
ACAGCGTGACAGCCGAGGACACCGCTACATACTATTGCGCCAGATTCCCC
CTGATCAACTACGACCCCTGGCTGGCTTATTGGGGCCAAGGCACCCTGGT
TACCGTGTCCGCC
SEQ ID NO: 43:
A140 VL nucleotide sequence
GACGTGCTGATGACCCAGACCCCTCTGAGCCTGCCTGTGTCCCTGGGCGA
TCAGGCCTCTATCTCCTGCAGAAGCTCTCAGTCTATCGTGCATAGCAACG
GCAATACCTACCTGGAATGGTATCTGCAAAAGCCTGGCCAGTCTCCTAAG
CTTCTGATCTACAAGGTGTCTAATCGGTTCAGCGGAGTCCCCGACCGCTT
CAGCGGCAGCGGCAGCGGCACCGACTTCACCCTGAAAATCTCAAGAGTGG
AAGCCGAGGATCTGGGCGTGTACTACTGCTTCCAGGGCAGCCACGTGCCT
TGGACCTTCGGCGGCGGCACAAAGCTGGAAATCAAT
SEQ ID NO: 44:
A142 VH nucleotide sequence
GAGGTGCAGCTGCAGCAGAGCGGTCCTGATCTGGTGAAACCTGGCGCTAG
CGTGAAGATCAGCTGCAAGGCCTCTGGATATTCTTTTACAGGCTACTACA
TGCACTGGGTCAAGCAGAGCCACGGCAAAAGCCTGGAGTGGATCGGCAGA
GTTTACCCCAACAACGGCGGCACAAGCTACAACCAGAAATTCAAGGGCAA
GGCTATCCTGACCGTGGACAAGTCCTCCACCACCGCCTACATGGAACTGC
GGAGCCTGACATCTGAGGATAGCGCCGTGTACTACTGCGCCCTGATTCAC
TACTACGGCTICTACAGCATGGACTACTGGGGCCAGGGCACCAGCGTGAC
AGTGTCCAGC
SEQ ID NO: 45:
A142 VL nucleotide sequence
GACGTGCTGATGACCCAGACCCCTCTGAGCCTGCCTGTGAGCCTCGGCGA
CCAGGCCAGCATCTCTTGTAGAAGCTCACAGTCTATCGTGCATAGCAACG
GAAATACCTACCTGGAATGGTATCTGCAAAAGCCCGGCCAATCCCCTAAG
CTGCTGATCTACAAGGTGTCTAATCGGTTTAGCGGAGTGCCCGATAGGTT
CAGCGGCAGCGGCAGCGGCACCGACTTCACCCTGAAGATCTCTAGAGTGG
AAGCCGAGGACCTGGGCGTGTACTATTGCTTCCAGGGCTCCCATGTGCCA
TGGACCTTCGGCGGCGGCACAAAGCTGGAGATCAAC
SEQ ID NO: 46:
A148 VH nucleotide sequence
GATGTGCAGCTGCAAGAGAGCGGCCCTGGCCTGGTCAAGCCTAGCCAAAG
CCTGAGCCTGACCTGCAGCGTTTCCGGCTACAGCATCACCTCTGGCTACT
ACTGGAACTGGATCAGACAGTTCCCTGGAAATAAACTGGAATGGATGGGA
TACATCAGCAATGACGGCTCTAACAAGTACAAGCCCAGCCTCAAGAACAG
AATCAGCATTACCCGCGACACCTCCAAGAACCAGTTTTTCCTGAAGCTGA
ACAGCGTGACCACAGAAGATACCGCTACATACTACTGCGCCAGATTCCCC
CTCATCAACTACGACCCCTGGCTGGCCTATTGGGGCCAGGGAACACTGGT
GACAGTGTCCGCC
SEQ ID NO: 47:
A148 VL nucleotide sequence
GACGTGCTGATGACCCAGACCCCTCTGTCTCTGCCTGTGTCCCTGGGCGA
CCAGGCCTCTATCAGCTGTAGAAGCAGCCAGAACGTGGTGCATAGAAACG
GCAACACCTACCTGGAGTGGTATCTGCAGAAACCTGGCCAGAGCCCTAAG
CTGCTGATCTACAAGGTGTCTAATCGGTTCAGCGGAGTGCCCGACCGGTT
TAGCGGCAGCGGCTCTGGAACAGATTTCACCCTGAAAATCAGCAGAGTGG
AAGCCGAGGACCTGGGCGTGTACTATTGCTTCCAGGGCAGCCACGTGCCA
TGGACATTCGGCGGTGGCACCAAGCTGGAGATCAAG
SEQ ID NO: 48:
A183 VH nucleotide sequence
GACGTGCAGCTGCAGGAGAGCGGTCCTGGCCTGGTCAAGCCTAGCCAGAG
CCTGAGCCTTACATGCAGCGTGTCCGGCTACAGCATCACCTCTGGATATT
ACTGGTCCTGGATCAGACAGTTTCCTGGCAACAAGCTGGAATGGATGGGC
TACATCAGCAACGACGGCTCTAACAAATACAAGCCCAGCCTGAAGAACCG
GATCAGCATTACAAGAGATACCAGCAAGAACCAGTTCTTCCTGAAACTGA
AAAGCGTCACCACCGAGGATACAGCTACATACTATTGTGCCAGATTCCCC
CTGATCAACTACGACCCCTGGCTGGCCTACTGGGGACAAGGCACCCTGGT
GACAGTGTCCGCC
SEQ ID NO: 49:
A183 VL nucleotide sequence
GACGTGCTGATGACCCAGACCCCTCTGTCTCTGCCTGTGTCCTTGGGCGA
CCAGGCCAGCATCTCATGCAGAAGCAGCCAGAACGTGGTGCATAGAAACG
GCAATACCTACCTGGAATGGTATCTGCAAAAGCCCGGCCAGTCTCCAAAG
CTGCTCATCTACAAGGTGTCTAATAGGTTTAGCGGCGTTCCTGATAGATT
CAGCGGCAGCGGCAGCGGCACAGACTTCACCCTGAAGATCTCTAGAGTGG
AAGCCGAGGACCTGGGCGTGTACTACTGCTTCCAGGGCAGCCACGTGCCT
TGGACCTTCGGCGGAGGAACCAAGCTGGAGATCAAG
SEQ ID NO: 50:
A129-8α-BB-ζ CAR nucleotide sequence
ATGGCCCTGCCTGTGACCGCCCTGCTGCTGCCTCTGGCCCTGCTGCTGCA
CGCCGCTCGCCCCGACGTGCTGATGACACAGGCCCCTCTGTCTCTGCCCG
TCAGCCTCGGCGACCAGGCCAGCATCAGCTGCAGAAGCAGCCAGAACGTG
GTGCATAGAAATGGCAATACCTACCTGGAATGGTATCTGCAGAAACCTGG
CCAATCTCCAAAGCTGCTGATCTACAAGGTGTCCAACAGATTCAGCGGCG
TGCCTGATAGATTCAGCGGCAGCGGATCTGGAACAGACTTCACCCTGAAG
ATCAGCAGAGTGGAAGCCGAGGATCTGGGCGTGTACTACTGCTTCCAGGG
CAGCCACGTGCCCTGGACCTTCGGCGGCGGCACCAAGCTCGAGATCAAGG
GTGGCGGCGGAAGCGGAGGCGGCGGCTCTGGCGGAGGCGGCAGCGACGTG
CAGCTGCAGGAGAGCGGACCTGGACTGGTTAAGCCTAGCCAAAGCCTGAG
CCTGACCTGCAGCGTGACAGGCTACAGCATCACCAGCGGGTATTACTGGA
ACTGGATCAGACAGTTCCCCGGCAACAAGCTGGAATGGATGGGCTACATC
AGAAACGACGGCTCCAACAACTACAACCCTTCTCTGAAAAACCGGATCTC
TATTACCAGAGATACCTCCAAGAACCAGTTTTTCCTGAAGCTGAATAGCG
TCACGACCGAGGACACAGCTACATACTATTGTGCTCGGTTTCCACTGATC
AACTACGACCCCTGGTTCGCCTACTGGGGCCAGGGCACCCTGGTGACGGT
GTCCGCCACAACCACCCCTGCCCCTAGACCTCCTACCCCTGCTCCAACCA
TCGCCAGCCAGCCCCTGTCTCTGCGGCCTGAAGCTTGCAGACCCGCTGCT
GGCGGCGCCGTGCACACGAGGGGACTGGACTTCGCCTGCGACATCTACAT
TTGGGCCCCTCTGGCCGGCACCTGCGGCGTGCTGCTGCTGAGCCTGGTCA
TCACACTGTACTGCAAGAGAGGCAGAAAAAAGCTGCTGTACATCTTCAAG
CAGCCTTTTATGCGGCCCGTGCAGACCACACAGGAGGAAGATGGCTGCTC
TTGTAGATTCCCCGAGGAAGAGGAAGGCGGATGTGAACTGAGAGTGAAGT
TCAGCAGAAGCGCCGATGCCCCTGCCTACCAGCAGGGACAGAACCAGCTG
TACAACGAGCTGAACCTGGGCAGACGGGAAGAGTACGACGTGCTGGATAA
GCGGCGGGGTCGGGACCCCGAGATGGGAGGCAAGCCTAGAAGGAAGAACC
CCCAAGAGGGCCTTTATAATGAGCTGCAGAAAGATAAGATGGCCGAAGCC
TACAGCGAGATCGGCATGAAGGGCGAGAGACGCAGAGGCAAAGGCCACGA
CGGCCTGTACCAAGGCCTCTCCACCGCCACAAAGGACACCTACGACGCCC
TGCACATGCAGGCCCTGCCTCCAAGATGA
SEQ ID NO: 51:
A129-8α-BB-ζ CAR amino acid sequence
MALPVTALLLPLALLLHAARPDVLMTQAPLSLPVSLGDQASISCRSSQNV
VHRNGNTYLEWYLQKPGQSPKLLIYKVSNRFSGVPDRFSGSGSGTDFTLK
ISRVEAEDLGVYYCFQGSHVPWTFGGGTKLEIKGGGGSGGGGSGGGGSDV
QLQESGPGLVKPSQSLSLTCSVTGYSITSGYYWNWIRQFPGNKLEWMGYI
RNDGSNNYNPSLKNRISITRDTSKNQFFLKLNSVTTEDTATYYCARFPLI
NYDPWFAYWGQGTLVTVSATTTPAPRPPTPAPTIASQPLSLRPEACRPAA
GGAVHTRGLDFACDIYIWAPLAGTCGVLLLSLVITLYCKRGRKKLLYIFK
QPFMRPVQTTQEEDGCSCRFPEEEEGGCELRVKFSRSADAPAYQQGQNQL
YNELNLGRREEYDVLDKRRGRDPEMGGKPRRKNPQEGLYNELQKDKMAEA
YSEIGMKGERRRGKGHDGLYQGLSTATKDTYDALHMQALPPR
SEQ ID NO: 52:
A129 CAR leader sequence
MALPVTALLLPLALLLHAARP
SEQ ID NO: 53:
A129 scFv
DVLMTQAPLSLPVSLGDQASISCRSSQNVVHRNGNTYLEWYLQKPGQSPK
LLIYKVSNRFSGVPDRFSGSGSGTDFTLKISRVEAEDLGVYYCFQGSHVP
WTFGGGTKLEIKGGGGSGGGGSGGGGSDVQLQESGPGLVKPSQSLSLTCS
VTGYSITSGYYWNWIRQFPGNKLEWMGYIRNDGSNNYNPSLKNRISITRD
TSKNQFFLKLNSVTTEDTATYYCARFPLINYDPWFAYWGQGTLVTVSA
SEQ ID NO: 54:
A129 scFv linker
GGGGSGGGGSGGGGS
SEQ ID NO: 55:
CD8α hinge
TTTPAPRPPTPAPTIASQPLSLRPEACRPAAGGAVHTRGLDFACD
SEQ ID NO: 56:
CD8α transmembrane domain
IYIWAPLAGTCGVLLLSLVITLYC
SEQ ID NO: 57:
4-1BB signaling domain
KRGRKKLLYIFKQPFMRPVQTTQEEDGCSCRFPEEEEGGCEL
SEQ ID NO: 58:
CD3ζ signaling domain
MALPVTALLLPLALLLHAARPDVLMTQAPLSLPVSLGDQASISCRSSQNV
VHRNGNTYLEWYLQKPGQSPKLLIYKVSNRFSGVPDRFSGSGSGTDFTLK
ISRVEAEDLGVYYCFQGSHVPWTFGGGTKLEIKGGGGSGGGGSGGGGSDV
QLQESGPGLVKPSQSLSLTCSVTGYSITSGYYWNWIRQFPGNKLEWMGYI
RNDGSNNYNPSLKNRISITRDTSKNQFFLKLNSVTTEDTATYYCARFPLI
NYDPWFAYWGQGTLVTVSATTTPAPRPPTPAPTIASQPLSLRPEACRPAA
GGAVHTRGLDFACDIYIWAPLAGTCGVLLLSLVITLYCKRGRKKLLYIFK
QPFMRPVQTTQEEDGCSCRFPEEEEGGCELRVKFSRSADAPAYQQGQNQL
YNELNLGRREEYDVLDKRRGRDPEMGGKPRRKNPQEGLYNELQKDKMAEA
YSEIGMKGERRRGKGHDGLYQGLSTATKDTYDALHMQALPPR
SEQ ID NO: 59:
A129-8α-OX40-ζ CAR nucleotide sequence
ATGGCCCTGCCTGTGACCGCCCTGCTGCTGCCTCTGGCCCTGCTGCTGCA
CGCCGCTCGCCCCGACGTGCTGATGACACAGGCCCCTCTGTCTCTGCCCG
TCAGCCTCGGCGACCAGGCCAGCATCAGCTGCAGAAGCAGCCAGAACGTG
GTGCATAGAAATGGCAATACCTACCTGGAATGGTATCTGCAGAAACCTGG
CCAATCTCCAAAGCTGCTGATCTACAAGGTGTCCAACAGATTCAGCGGCG
TGCCTGATAGATTCAGCGGCAGCGGATCTGGAACAGACTTCACCCTGAAG
ATCAGCAGAGTGGAAGCCGAGGATCTGGGCGTGTACTACTGCTTCCAGGG
CAGCCACGTGCCCTGGACCTTCGGCGGCGGCACCAAGCTCGAGATCAAGG
GTGGCGGCGGAAGCGGAGGCGGCGGCTCTGGCGGAGGCGGCAGCGACGTG
CAGCTGCAGGAGAGCGGACCTGGACTGGTTAAGCCTAGCCAAAGCCTGAG
CCTGACCTGCAGCGTGACAGGCTACAGCATCACCAGCGGGTATTACTGGA
ACTGGATCAGACAGTTCCCCGGCAACAAGCTGGAATGGATGGGCTACATC
AGAAACGACGGCTCCAACAACTACAACCCTTCTCTGAAAAACCGGATCTC
TATTACCAGAGATACCTCCAAGAACCAGTTTTTCCTGAAGCTGAATAGCG
TCACGACCGAGGACACAGCTACATACTATTGTGCTCGGTTTCCACTGATC
AACTACGACCCCTGGTTCGCCTACTGGGGCCAGGGCACCCTGGTGACCGT
GTCCGCCACAACCACCCCTGCCCCTAGACCTCCTACCCCTGCTCCAACCA
TCGCCAGCCAGCCCCTGTCTCTGCGGCCTGAAGCTTGCAGACCCGCTGCT
GGCGGCGCCGTGCACACGAGGGGACTGGACTTCGCCTGCGACATCTACAT
TTGGGCCCCTCTGGCCGGCACCTGCGGCGTGCTGCTGCTGAGCCTGGTCA
TCACACTGTACTGCCGGAGGGACCAGAGGCTGCCCCCCGATGCCCACAAG
CCCCCTGGGGGAGGCAGTTTCCGGACCCCCATCCAAGAGGAGCAGGCCGA
CGCCCACTCCACCCTGGCCAAGATCAGAGTGAAGTTCAGCAGAAGCGCCG
ATGCCCCTGCCTACCAGCAGGGACAGAACCAGCTGTACAACGAGCTGAAC
CTGGGCAGACGGGAAGAGTACGACGTGCTGGATAAGCGGCGGGGTCGGGA
CCCCGAGATGGGAGGCAAGCCTAGAAGGAAGAACCCCCAAGAGGGCCTTT
ATAATGAGCTGCAGAAAGATAAGATGGCCGAAGCCTACAGCGAGATCGGC
ATGAAGGGCGAGAGACGCAGAGGCAAAGGCCACGACGGCCTGTACCAAGG
CCTCTCCACCGCCACAAAGGACACCTACGACGCCCTGCACATGCAGGCCC
TGCCTCCAAGATGA
SEQ ID NO: 60:
A129-8α-OX40-ζ CAR amino acid sequence
MALPVTALLLPLALLLHAARPDVLMTQAPLSLPVSLGDQASISCRSSQNV
VHRNGNTYLEWYLQKPGQSPKLLIYKVSNRFSGVPDRFSGSGSGTDFTLK
ISRVEAEDLGVYYCFQGSHVPWTFGGGTKLEIKGGGGSGGGGSGGGGSDV
QLQESGPGLVKPSQSLSLTCSVTGYSITSGYYWNWIRQFPGNKLEWMGYI
RNDGSNNYNPSLKNRISITRDTSKNQFFLKLNSVTTEDTATYYCARFPLI
NYDPWFAYWGQGTLVTVSATTTPAPRPPTPAPTIASQPLSLRPEACRPAA
GGAVHTRGLDFACDIYIWAPLAGTCGVLLLSLVITLYCRRDQRLPPDAHK
PPGGGSFRTPIQEEQADAHSTLAKIRVKFSRSADAPAYQQGQNQLYNELN
LGRREEYDVLDKRRGRDPEMGGKPRRKNPQEGLYNELQKDKMAEAYSEIG
MKGERRRGKGHDGLYQGLSTATKDTYDALHMQALPPR
SEQ ID NO: 61:
A129-28-28-ζ CAR nucleotide sequence
ATGGCCCTGCCTGTGACCGCCCTGCTGCTGCCTCTGGCCCTGCTGCTGCA
CGCCGCTCGCCCCGACGTGCTGATGACACAGGCCCCTCTGTCTCTGCCCG
TCAGCCTCGGCGACCAGGCCAGCATCAGCTGCAGAAGCAGCCAGAACGTG
GTGCATAGAAATGGCAATACCTACCTGGAATGGTATCTGCAGAAACCTGG
CCAATCTCCAAAGCTGCTGATCTACAAGGTGTCCAACAGATTCAGCGGCG
TGCCTGATAGATTCAGCGGCAGCGGATCTGGAACAGACTTCACCCTGAAG
ATCAGCAGAGTGGAAGCCGAGGATCTGGGCGTGTACTACTGCTTCCAGGG
CAGCCACGTGCCCTGGACCTTCGGCGGCGGCACCAAGCTCGAGATCAAGG
GTGGCGGCGGAAGCGGAGGCGGCGGCTCTGGCGGAGGCGGCAGCGACGTG
CAGCTGCAGGAGAGCGGACCTGGACTGGTTAAGCCTAGCCAAAGCCTGAG
CCTGACCTGCAGCGTGACAGGCTACAGCATCACCAGCGGGTATTACTGGA
ACTGGATCAGACAGTTCCCCGGCAACAAGCTGGAATGGATGGGCTACATC
AGAAACGACGGCTCCAACAACTACAACCCTTCTCTGAAAAACCGGATCTC
TATTACCAGAGATACCTCCAAGAACCAGTTTTTCCTGAAGCTGAATAGCG
TCACGACCGAGGACACAGCTACATACTATTGTGCTCGGTTTCCACTGATC
AACTACGACCCCTGGTTCGCCTACTGGGGCCAGGGCACCCTGGTGACGGT
GAGCGCTATTGAAGTGATGTACCCACCACCATACCTGGACAACGAGAAGA
GCAACGGGACTATTATCCATGTGAAGGGGAAACACCTGTGCCCAAGCCCA
CTGTTCCCCGGCCCTTCCAAGCCCTTTTGGGTGCTGGTGGTGGTGGGCGG
CGTCCTGGCTTGTTACTCTCTGCTGGTCACTGTGGCTTTCATCATCTTCT
GGGTCAAGAGGAGCCGCCTGCTGCACTCCGACTACATGAACATGACCCCT
AGACGGCCCGGACCAACAAGGAAGCACTACCAGCCCTATGCCCCCCCTAG
GGATTTCGCCGCCTATAGAGTGAAGTTCAGCAGAAGCGCCGATGCCCCTG
CCTACCAGCAGGGACAGAACCAGCTGTACAACGAGCTGAACCTGGGCAGA
CGGGAAGAGTACGACGTGCTGGATAAGCGGCGGGGTCGGGACCCCGAGAT
GGGAGGCAAGCCTAGAAGGAAGAACCCCCAAGAGGGCCTTTATAATGAGC
TGCAGAAAGATAAGATGGCCGAAGCCTACAGCGAGATCGGCATGAAGGGC
GAGAGACGCAGAGGCAAAGGCCACGACGGCCTGTACCAAGGCCTCTCCAC
CGCCACAAAGGACACCTACGACGCCCTGCACATGCAGGCCCTGCCTCCAA
GAT
SEQ ID NO: 62:
A129-28-28-2 CAR amino acid sequence
MALPVTALLLPLALLLHAARPDVLMTQAPLSLPVSLGDQASISCRSSQNV
VHRNGNTYLEWYLQKPGQSPKLLIYKVSNRFSGVPDRFSGSGSGTDFTLK
ISRVEAEDLGVYYCFQGSHVPWTFGGGTKLEIKGGGGSGGGGSGGGGSDV
QLQESGPGLVKPSQSLSLTCSVTGYSITSGYYWNWIRQFPGNKLEWMGYI
RNDGSNNYNPSLKNRISITRDTSKNQFFLKLNSVTTEDTATYYCARFPLI
NYDPWFAYWGQGTLVTVSAIEVMYPPPYLDNEKSNGTIIHVKGKHLCPSP
LFPGPSKPFWVLVVVGGVLACYSLLVTVAFIIFWVKRSRLLHSDYMNMTP
RRPGPTRKHYQPYAPPRDFAAYRVKFSRSADAPAYQQGQNQLYNELNLGR
REEYDVLDKRRGRDPEMGGKPRRKNPQEGLYNELQKDKMAEAYSEIGMKG
ERRRGKGHDGLYQGLSTATKDTYDALHMQALPPR
SEQ ID NO: 63:
A129-28-OX40-ζ CAR nucleotide sequence
ATGGCCCTGCCTGTGACCGCCCTGCTGCTGCCTCTGGCCCTGCTGCTGCA
CGCCGCTCGCCCCGACGTGCTGATGACACAGGCCCCTCTGTCTCTGCCCG
TCAGCCTCGGCGACCAGGCCAGCATCAGCTGCAGAAGCAGCCAGAACGTG
GTGCATAGAAATGGCAATACCTACCTGGAATGGTATCTGCAGAAACCTGG
CCAATCTCCAAAGCTGCTGATCTACAAGGTGTCCAACAGATTCAGCGGCG
TGCCTGATAGATTCAGCGGCAGCGGATCTGGAACAGACTTCACCCTGAAG
ATCAGCAGAGTGGAAGCCGAGGATCTGGGCGTGTACTACTGCTTCCAGGG
CAGCCACGTGCCCTGGACCTTCGGCGGCGGCACCAAGCTCGAGATCAAGG
GTGGCGGCGGAAGCGGAGGCGGCGGCTCTGGCGGAGGCGGCAGCGACGTG
CAGCTGCAGGAGAGCGGACCTGGACTGGTTAAGCCTAGCCAAAGCCTGAG
CCTGACCTGCAGCGTGACAGGCTACAGCATCACCAGCGGGTATTACTGGA
ACTGGATCAGACAGTTCCCCGGCAACAAGCTGGAATGGATGGGCTACATC
AGAAACGACGGCTCCAACAACTACAACCCTTCTCTGAAAAACCGGATCTC
TATTACCAGAGATACCTCCAAGAACCAGTTTTTCCTGAAGCTGAATAGCG
TCACGACCGAGGACACAGCTACATACTATTGTGCTCGGTTTCCACTGATC
AACTACGACCCCTGGTTCGCCTACTGGGGCCAGGGCACCCTGGTGACGGT
GAGCGCTATTGAAGTGATGTACCCACCACCATACCTGGACAACGAGAAGA
GCAACGGGACTATTATCCATGTGAAGGGGAAACACCTGTGCCCAAGCCCA
CTGTTCCCCGGCCCTTCCAAGCCCTTTTGGGTGCTGGTGGTGGTGGGCGG
CGTCCTGGCTTGTTACTCTCTGCTGGTCACTGTGGCTTTCATCATCTTCT
GGGTCCGGAGGGACCAGAGGCTGCCCCCCGATGCCCACAAGCCCCCTGGG
GGAGGCAGTTTCCGGACCCCCATCCAAGAGGAGCAGGCCGACGCCCACTC
CACCCTGGCCAAGATCAGAGTGAAGTTCAGCAGAAGCGCCGATGCCCCTG
CCTACCAGCAGGGACAGAACCAGCTGTACAACGAGCTGAACCTGGGCAGA
CGGGAAGAGTACGACGTGCTGGATAAGCGGCGGGGTCGGGACCCCGAGAT
GGGAGGCAAGCCTAGAAGGAAGAACCCCCAAGAGGGCCTTTATAATGAGC
TGCAGAAAGATAAGATGGCCGAAGCCTACAGCGAGATCGGCATGAAGGGC
GAGAGACGCAGAGGCAAAGGCCACGACGGCCTGTACCAAGGCCTCTCCAC
CGCCACAAAGGACACCTACGACGCCCTGCACATGCAGGCCCTGCCTCCAA
GAT
SEQ ID NO: 64:
A129-28-OX40-ζ CAR amino acid sequence
MALPVTALLLPLALLLHAARPDVLMTQAPLSLPVSLGDQASISCRSSQNV
VHRNGNTYLEWYLQKPGQSPKLLIYKVSNRFSGVPDRFSGSGSGTDFTLK
ISRVEAEDLGVYYCFQGSHVPWTFGGGTKLEIKGGGGSGGGGSGGGGSDV
QLQESGPGLVKPSQSLSLTCSVTGYSITSGYYWNWIRQFPGNKLEWMGYI
RNDGSNNYNPSLKNRISITRDTSKNQFFLKLNSVTTEDTATYYCARFPLI
NYDPWFAYWGQGTLVTVSAIEVMYPPPYLDNEKSNGTIIHVKGKHLCPSP
LFPGPSKPFWVLVVVGGVLACYSLLVTVAFIIFWVRRDQRLPPDAHKPPG
GGSFRTPIQEEQADAHSTLAKIRVKFSRSADAPAYQQGQNQLYNELNLGR
REEYDVLDKRRGRDPEMGGKPRRKNPQEGLYNELQKDKMAEAYSEIGMKG
ERRRGKGHDGLYQGLSTATKDTYDALHMQALPPR
SEQ ID NO: 65:
A129-28-BB-ζ CAR nucleotide sequence
ATGGCCCTGCCTGTGACCGCCCTGCTGCTGCCTCTGGCCCTGCTGCTGCA
CGCCGCTCGCCCCGACGTGCTGATGACACAGGCCCCTCTGTCTCTGCCCG
TCAGCCTCGGCGACCAGGCCAGCATCAGCTGCAGAAGCAGCCAGAACGTG
GTGCATAGAAATGGCAATACCTACCTGGAATGGTATCTGCAGAAACCTGG
CCAATCTCCAAAGCTGCTGATCTACAAGGTGTCCAACAGATTCAGCGGCG
TGCCTGATAGATTCAGCGGCAGCGGATCTGGAACAGACTTCACCCTGAAG
ATCAGCAGAGTGGAAGCCGAGGATCTGGGCGTGTACTACTGCTTCCAGGG
CAGCCACGTGCCCTGGACCTTCGGCGGCGGCACCAAGCTCGAGATCAAGG
GTGGCGGCGGAAGCGGAGGCGGCGGCTCTGGCGGAGGCGGCAGCGACGTG
CAGCTGCAGGAGAGCGGACCTGGACTGGTTAAGCCTAGCCAAAGCCTGAG
CCTGACCTGCAGCGTGACAGGCTACAGCATCACCAGCGGGTATTACTGGA
ACTGGATCAGACAGTTCCCCGGCAACAAGCTGGAATGGATGGGCTACATC
AGAAACGACGGCTCCAACAACTACAACCCTTCTCTGAAAAACCGGATCTC
TATTACCAGAGATACCTCCAAGAACCAGTTTTTCCTGAAGCTGAATAGCG
TCACGACCGAGGACACAGCTACATACTATTGTGCTCGGTTTCCACTGATC
AACTACGACCCCTGGTTCGCCTACTGGGGCCAGGGCACCCTGGTGACGGT
GAGCGCTATTGAAGTGATGTACCCACCACCATACCTGGACAACGAGAAGA
GCAACGGGACTATTATCCATGTGAAGGGGAAACACCTGTGCCCAAGCCCA
CTGTTCCCCGGCCCTTCCAAGCCCTTTTGGGTGCTGGTGGTGGTGGGCGG
CGTCCTGGCTTGTTACTCTCTGCTGGTCACTGTGGCTTTCATCATCTTCT
GGGTCAAGAGAGGCAGAAAAAAGCTGCTGTACATCTTCAAGCAGCCTTTT
ATGCGGCCCGTGCAGACCACACAGGAGGAAGATGGCTGCTCTTGTAGATT
CCCCGAGGAAGAGGAAGGCGGATGTGAACTGAGAGTGAAGTTCAGCAGAA
GCGCCGATGCCCCTGCCTACCAGCAGGGACAGAACCAGCTGTACAACGAG
CTGAACCTGGGCAGACGGGAAGAGTACGACGTGCTGGATAAGCGGCGGGG
TCGGGACCCCGAGATGGGAGGCAAGCCTAGAAGGAAGAACCCCCAAGAGG
GCCTTTATAATGAGCTGCAGAAAGATAAGATGGCCGAAGCCTACAGCGAG
ATCGGCATGAAGGGCGAGAGACGCAGAGGCAAAGGCCACGACGGCCTGTA
CCAAGGCCTCTCCACCGCCACAAAGGACACCTACGACGCCCTGCACATGC
AGGCCCTGCCTCCAAGATGA
SEQ ID NO: 66:
A129-28-BB-ζ CAR amino acid sequence
MALPVTALLLPLALLLHAARPDVLMTQAPLSLPVSLGDQASISCRSSQNV
VHRNGNTYLEWYLQKPGQSPKLLIYKVSNRFSGVPDRFSGSGSGTDFTLK
ISRVEAEDLGVYYCFQGSHVPWTFGGGTKLEIKGGGGSGGGGSGGGGSDV
QLQESGPGLVKPSQSLSLTCSVTGYSITSGYYWNWIRQFPGNKLEWMGYI
RNDGSNNYNPSLKNRISITRDTSKNQFFLKLNSVTTEDTATYYCARFPLI
NYDPWFAYWGQGTLVTVSAIEVMYPPPYLDNEKSNGTIIHVKGKHLCPSP
LFPGPSKPFWVLVVVGGVLACYSLLVTVAFIIFWVKRGRKKLLYIFKQPF
MRPVQTTQEEDGCSCRFPEEEEGGCELRVKFSRSADAPAYQQGQNQLYNE
LNLGRREEYDVLDKRRGRDPEMGGKPRRKNPQEGLYNELQKDKMAEAYSE
IGMKGERRRGKGHDGLYQGLSTATKDTYDALHMQALPPR

What is claimed is:

1. An isolated antibody or antigen-binding portion thereof comprising:
(a) a heavy chain variable region comprising
(i) a CDRH1 comprising SEQ ID NOs: 12 or 14;
(ii) a CDRH2 comprising SEQ ID NOs: 15, 17, 18, or 19; and
(iii) a CDRH3 comprising SEQ ID NOs: 20, 22, or 23; and
(b) a light chain variable region comprising
(i) a CDRL1 comprising SEQ ID NOs: 24, 26, or 27;
(ii) a CDRL2 comprising SEQ ID NOs: 28 or 30; and
(iii) a CDRL3 comprising SEQ ID NOs: 31 or 33.

2. An isolated antibody or antigen-binding portion thereof comprising:
(a) a heavy chain variable region comprising
(i) a CDRH1 comprising SEQ ID NO: 13;
(ii) a CDRH2 comprising SEQ ID NO: 16; and
(iii) a CDRH3 comprising SEQ ID NO: 21; and
(b) a light chain variable region comprising
(i) a CDRL1 comprising SEQ ID NO: 25;
(ii) a CDRL2 comprising SEQ ID NO: 29; and
(iii) a CDRL3 comprising SEQ ID NO: 32.

3. An isolated antibody or antigen binding portion thereof comprising:
(a) a heavy chain variable region comprising an amino acid sequence that is at least 90% identical to SEQ ID NO:1; and
(b) a light chain variable region comprising an amino acid sequence that is at least 90% identical to SEQ ID NOs: 7 or 11.

4. An isolated antibody or antigen binding portion thereof comprising:
(a) a heavy chain variable region comprising an amino acid sequence that is at least 90% identical to SEQ ID NO:2; and
(b) a light chain variable region comprising an amino acid sequence that is at least 90% identical to SEQ ID NO:8.

5. An isolated antibody or antigen binding portion thereof comprising:
(a) a heavy chain variable region comprising an amino acid sequence that is at least 90% identical to SEQ ID NO:3; and
(b) a light chain variable region comprising an amino acid sequence that is at least 90% identical to SEQ ID NOs: 9 or 10.

6. An isolated antibody or antigen binding portion thereof comprising:
(a) a heavy chain variable region comprising an amino acid sequence that is at least 90% identical to SEQ ID NOs: 4 or 5; and
(b) a light chain variable region comprising an amino acid sequence that is at least 90% identical to SEQ ID NO:7.

7. An isolated antibody or antigen binding portion thereof comprising:
(a) a heavy chain variable region comprising an amino acid sequence that is at least 90% identical to SEQ ID NO:6; and
(b) a light chain variable region comprising an amino acid sequence that is at least 90% identical to SEQ ID NO:11.

8. A chimeric antigen receptor (CAR) comprising:
(a) an extracellular target-binding domain comprising an antibody or antigen binding portion thereof of any of claims 1 to 7;
(b) a transmembrane domain; and
(c) a signaling domain.

9. The CAR of claim 8, wherein the antibody or antigen binding portion thereof is a single chain antibody fragment, a single chain Fv (scFv), a single chain Fab, a single chain Fab′, a single domain antibody fragment, a single domain multispecific antibody, an intrabody, a nanobody, or a single chain immunokine.

10. The CAR of claim 8 or 9, wherein the antibody or antigen binding portion thereof is a scFv comprising an amino acid sequence that is at least 90% identical to SEQ ID NO:53.

11. The CAR of any of claims 8 to 10, wherein the hinge and transmembrane domains comprise CD8α or CD28 hinge and transmembrane domains.

12. The CAR of any of claims 8 to 11, wherein the signaling domain comprises a 4-1BB signaling domain, a CD28 signaling domain, an OX-40 signaling domain, and/or a CD3ζ signaling domain.

13. The CAR of any of claims 8 to 12, wherein the CAR further comprises a leader sequence and/or a hinge domain.

14. The CAR of any of claims 8 to 13, wherein the CAR comprises an amino acid sequence that is at least 90% identical to SEQ ID NOs: 51, 60, 62, 64, or 66.

15. The CAR of any of claims 8 to 14, wherein the extracellular target-binding domain further comprises one or more additional antigen-binding domains.

16. The CAR of claim 15, wherein the one or more additional antigen-binding domains specifically bind to CD19, CD20, CD22, CD79a, CD79b, or any combination thereof.

17. A recombinant nucleic acid molecule encoding an antibody or antigen binding portion thereof of any of claims 1 to 7 or a CAR of any of claims 8 to 16.

18. The recombinant nucleic acid molecule of claim 17, wherein said recombinant nucleic acid molecule is a synthetic sequence designed for expression in a host cell.

19. A DNA construct comprising the recombinant nucleic acid molecule of claim 17 or 18 operably linked to a promoter that drives expression in a host cell.

20. A vector comprising the recombinant nucleic acid molecule of claim 17 or 18 or the DNA construct of claim 19.

21. A host cell comprising the recombinant nucleic acid molecule of claim 17 or 18, the DNA construct of claim 19, or the vector of claim 20.

22. The host cell of claim 21, wherein said host cell is a bacterial cell.

23. The host cell of claim 21, wherein said host cell is a eukaryotic cell.

24. The host cell of claim 21, wherein said host cell is an immune effector cell.

25. The host cell of claim 24, wherein said immune effector cell is a T cell.

26. A composition comprising (a) an antibody or antigen binding portion thereof of any of claims 1 to 7 or a CAR of any of claims 8 to 16; and (b) a pharmaceutically acceptable carrier.

27. A method of detecting a presence of ROR1 in a biological sample, the method comprising:
(a) contacting said biological sample with the isolated antibody or antigen binding portion thereof of any of claims 1 to 7, and
(b) detecting an amount of binding of the isolated antibody or antigen binding portion thereof as a determination of the presence of ROR1 in the biological sample.

28. The method of claim 27, wherein the biological sample comprises cancer cells.

29. The method of claim 27, wherein the biological sample comprises a tumor sample of a tumor from a subject.

30. A method of treating a cancer of a subject, the method comprising administering to the subject a pharmaceutically effective amount of the composition of claim 26.

31. The method of claim 30, wherein the cancer is a ROR1-expressing cancer.

32. The method of claim 30 or 31, wherein the cancer comprises at least one of a lymphoma, a leukemia, or a solid tumor cancer.

33. The method of claim 32, wherein the lymphoma is follicular lymphoma, mantle cell lymphoma, diffuse large B-cell lymphoma, or marginal zone lymphoma.

34. The method of claim 32, wherein the leukemia is chronic lymphocytic leukemia.

35. The method of claim 32, wherein the solid tumor cancer is breast cancer, ovarian cancer, colon cancer, lung cancer, skin cancer, pancreatic cancer, testicular cancer, bladder cancer, uterus cancer, prostate cancer, or adrenal cancer.

36. The method of any one of claims 30 to 35, wherein the isolated antibody or antigen binding portion thereof is conjugated to a therapeutic agent.

37. The method of claim 36, wherein the therapeutic agent is at least one of a cytotoxic agent, a chemotherapeutic agent, or an immunosuppressive agent.

38. The method of claim 36 or 37, wherein the therapeutic agent is a moiety that specifically binds to an immune cell.

39. The method of claim 38, wherein the immune cell is a T cell.

40. The method of claim 38, wherein the immune cell is a natural killer cell.

41. The method of any one of claims 30 to 40, wherein the method further comprises administering a second form of cancer therapy to the subject.

42. The method of claim 41, wherein the second form of cancer therapy comprises a cytotoxic agent, a chemotherapeutic agent, an immunosuppressive agent, or radiation therapy.

43. A method of imaging a tumor in a subject with a ROR1-expressing cancer, the method comprising:
(a) administering to the subject the isolated antibody or antigen binding portion thereof of any one of claims 1 to 7 conjugated to an imaging label, and
(b) detecting the imaging label in the subject to obtain an image of the tumor.

44. A method of monitoring a response of a subject to a cancer therapy, wherein the subject has a ROR1-expressing cancer, the method comprising:
(a) administering to the subject, at a first time point before the subject receives the cancer therapy, the isolated antibody or antigen binding portion thereof of any one of claims 1 to 7 conjugated to an imaging label;
(b) detecting the imaging label in the subject to obtain a first image of a tumor in the subject;
(c) administering to the subject, at a second time point after the subject receives the cancer therapy, the isolated antibody or antigen binding portion thereof conjugated to the imaging label;
(d) detecting the imaging label in the subject to obtain a second image of the tumor; and
(e) comparing the first image to the second image to determine whether a change in tumor size has occurred.

45. The method of claim 44, wherein steps (c) to (e) are repeated at a third time point after the subject receives the cancer therapy.

46. The method of any one of claims 43 to 45, wherein the imaging label comprises a radioisotope, a bioluminescent label, a chemiluminescent label, or a paramagnetic compound.

47. A method of assessing responsiveness of a subject to a treatment with a ROR1 targeted therapy, wherein the subject has a cancer, the method comprising:
(a) measuring an amount of ROR1 in a tumor sample from the subject;
(b) determining, based on the amount of ROR1, if the cancer is characterized as having a high level of ROR1 expression; and
(c) indicating that the subject is more likely to respond to the treatment if the cancer is characterized as having the high level of ROR1 expression or that the subject is less likely to respond to the treatment if the cancer is characterized as having a low level of ROR1 expression,
wherein at least one of (i) the ROR1 targeted therapy comprises administration of the composition of claim 26, or (ii) the amount of ROR1 in the tumor sample is measured using the isolated antibody or antigen binding portion thereof of any one of claims 1 to 7.
